(19) United States
(12) Patent Application Publication
(10) Pub. No.: US 2022/0177424 A1
(43) Pub. Date: Jun. 9, 2022

(54) RADIATION-SENSITIVE RESIN COMPOSITION AND METHOD FOR FORMING RESIST PATTERN

(71) Applicant: JSR CORPORATION, Tokyo (JP)

(72) Inventors: Ryuichi NEMOTO, Tokyo (JP); Katsuaki Nishikori, Tokyo (JP); Ryosuke Nakamura, Tokyo (JP); Takuhiro Taniguchi, Tokyo (JP)

(73) Assignee: JSR CORPORATION, Tokyo (JP)

(21) Appl. No.: 17/679,262

(22) Filed: Feb. 24, 2022

Related U.S. Application Data
(63) Continuation-in-part of application No. PCT/JP2020/030051, filed on Aug. 5, 2020.

(30) Foreign Application Priority Data
Aug. 29, 2019 (JP) 2019-157261

(51) Int. Cl. C07C 381/12; G03F 7/20; G03F 7/38; G03F 7/16; G03F 7/038; G03F 7/32

(52) U.S. Cl. CPC C07C 381/12; G03F 7/2006; G03F 7/322; G03F 7/162; G03F 7/0382; G03F 7/38

(57) ABSTRACT

A radiation-sensitive resin composition contains: an onium salt compound represented by formula (1); a resin containing a structural unit having an acid-dissociable group; and a solvent. R¹ is a substituted or unsubstituted monovalent organic group having a cyclic structure, or a chain hydrocarbon group having 2 or more carbon atoms. X is an oxygen atom, a sulfur atom, or —NR^{α}—. R^{α} is a hydrogen atom or a monovalent hydrocarbon group having 1 to 10 carbon atoms. Z⁺ is a monovalent onium cation.

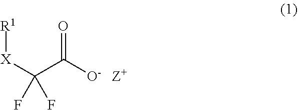

### CROSS-REFERENCE TO RELATED APPLICATIONS

The present application is a continuation-in-part application of International Application No. PCT/JP2020/030051, filed Aug. 5, 2020, which claims priority to Japanese Patent Application No. 2019-157261, filed Aug. 29, 2019. The contents of these applications are incorporated herein by reference in their entirety.

### BACKGROUND OF THE INVENTION

### Field of the Invention

The present invention relates to a radiation-sensitive resin composition and a method for forming a resist pattern.

### Description of the Related Art

A photolithography technology using a resist composition has been used for the fine circuit formation in a semiconductor device. As the representative procedure, for example, a resist pattern is formed on a substrate by generating an acid by irradiating the coating of the resist composition with a radioactive ray through a mask pattern, and then reacting in the presence of the acid as a catalyst to generate the difference of solubility of a resin into an alkaline or organic developer between an exposed part and a non-exposed part.

In the photolithography technique, the micronization of the pattern is promoted by using a short-wavelength radioactive ray such as an ArF excimer laser or by using an immersion exposure method (liquid immersion lithography) in which exposure is performed in a state in which a space between a lens of an exposure apparatus and a resist film is filled with a liquid medium.

While efforts for further technological development are in progress, a technique has been proposed in which a photosensitive quencher is blended in a resist composition, and an acid diffused to a non-exposed part is captured by an ion exchange reaction to improve lithographic performance with ArF exposure (see, JP-A-2015-54833). In addition, as a next-generation technology, lithography using a shorter-wavelength radioactive ray such as an electron beam, an X-ray, and extreme ultraviolet (EUV) is also being studied.

### SUMMARY OF THE INVENTION

According to an aspect of the present invention, a radiation-sensitive resin composition contains: an onium salt compound represented by formula (1); a resin containing a structural unit having an acid-dissociable group; and a solvent. R¹ is a substituted or unsubstituted monovalent organic group having a cyclic structure, or a chain hydrocarbon group having 2 or more carbon atoms. X is an oxygen atom, a sulfur atom, or —NR^{α}—. R^{α} is a hydrogen atom or a monovalent hydrocarbon group having 1 to 10 carbon atoms. Z⁺ is a monovalent onium cation.

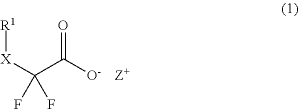

According to another aspect of the present invention, a method for forming a resist pattern, includes forming a resist film directly or indirectly on a substrate by applying the above-mentioned radiation-sensitive resin composition. The resist film is exposed. The exposed resist film is developed.

### DETAILED DESCRIPTION OF THE PREFERRED EMBODIMENTS

Among such efforts for the next-generation technology, various resist performances equal to or higher than ever before are required in terms of sensitivity, critical dimension uniformity (CDU) performance, which is an index of uniformity of sensitivity, line width, and hole diameter, and line width uniformity (LWR) performance indicating variation in line width of a resist pattern.

An embodiment of the present invention relates to a radiation-sensitive resin composition including:

an onium salt compound (hereinafter, also referred to as “compound (1)”) represented by formula (1) below:

a resin containing a structural unit having an acid-dissociable group; and

a solvent:

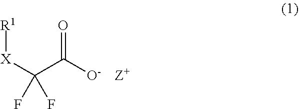

(wherein R¹ is a substituted or unsubstituted monovalent organic group having a cyclic structure or a chain hydrocarbon group having 2 or more carbon atoms,

X is an oxygen atom, a sulfur atom, or —NR^{α}—, wherein R^{α} is a hydrogen atom or a monovalent hydrocarbon group having 1 to 10 carbon atoms, and

Z⁺ is a monovalent onium cation).

The radiation-sensitive resin composition contains the compound (1) as a quencher (acid diffusion controlling agent), and thus can exhibit excellent sensitivity, CDU performance, and LWR performance in the formation of a resist pattern. Although not bound by any theory, the reason for this is presumed as follows. One is that two electron-withdrawing fluorine atoms are bonded to a carbon atom adjacent to the carboxylate ion, and an electron-withdrawing (thio)ether bond or a nitrogen atom is similarly disposed adjacent to the carbon atom, whereby (the anion moiety of) the compound (1) exhibits moderate weak basicity and promotes salt exchange with the generated acid. Further, introducing a cyclic structure or a chain hydrocarbon structure having a predetermined length into the anion moiety of the compound (1) suppresses the diffusion length of the compound (1) itself. It is presumed that these actions improve the acid diffusion controlling ability in the non-exposed part of the compound (1), and thus allow excellent various resist performances to be exhibited. The organic group refers to a group containing at least one carbon atom.

Another embodiment of the present invention relates to a method for forming a resist pattern, including:

applying the radiation-sensitive resin composition directly or indirectly on a substrate to form a resist film;

exposing the resist film; and

developing the exposed resist film.

The method for forming a resist pattern uses the above-described radiation-sensitive resin composition excellent in sensitivity, CDU performance, and LWR performance, and therefore a high-quality resist pattern can efficiently be formed.

Hereinbelow, embodiments of the present invention will be described in detail, but the present invention is not limited to these embodiments.

### <Radiation-Sensitive Resin Composition>

The radiation-sensitive resin composition (hereinafter, also simply referred to as “composition”) according to the present embodiment contains a compound (1), a resin, and a solvent. The radiation-sensitive resin composition further contains a radiation-sensitive acid generator, as necessary. The composition may contain other optional components as long as the effects of the present invention are not impaired.

### (Compound (1))

The compound (1) can function as a quencher (also referred to as a “photodegradable base” or “acid diffusion controlling agent”) that captures acid before exposure or acid in a non-exposed part. The compound (1) is represented by the following formula (1).

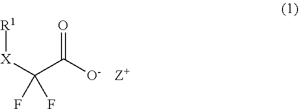

(wherein R¹ is a substituted or unsubstituted monovalent organic group having a cyclic structure or a chain hydrocarbon group having 2 or more carbon atoms,

X is an oxygen atom, a sulfur atom, or —NR^{α}—, wherein R^{α} is a hydrogen atom or a monovalent hydrocarbon group having 1 to 10 carbon atoms, and

Z⁺ is a monovalent onium cation).

When the compound (1) is contained, high levels of sensitivity, CDU performance, and LWR performance can be imparted to the radiation-sensitive resin composition.

In the above formula (1), the substituted or unsubstituted monovalent organic group having a cyclic structure is not particularly limited, and may be either a group containing only a cyclic structure or a group combining a cyclic structure and a chain structure. The cyclic structure may be either monocyclic or polycyclic. The cyclic structure may be any of an aromatic ring structure, an alicyclic structure, a heterocyclic structure, or a combination thereof. The heterocyclic structure includes a lactone structure, a cyclic carbonate structure, a sultone structure, or a combination thereof. These structures are preferably included as the smallest basic skeleton of the cyclic structure. The number of cyclic structures as the basic skeleton in the organic group may be one or two or more. A heteroatom may be interposed between carbon atoms forming the skeleton of the cyclic structure or the chain structure. A hydrogen atom on the carbon atom of the cyclic structure or the chain structure may be substituted with another substituent. When R¹ has a cyclic structure, the affinity of the compound (1) with the resin can be enhanced, and the diffusion length of the compound (1) can be moderately suppressed. As a result, various resist performances of the radiation-sensitive resin composition can be improved.

Examples of the aromatic ring structure as a basic skeleton include benzene, indene, naphthalene, azulene, phenanthrene, anthracene, tetracene, tetraphene, chrysene, pyrene, pentacene, triphenylene, and fluorene.

Among them, the aromatic ring structure is preferably at least one selected from the group consisting of the following structures.

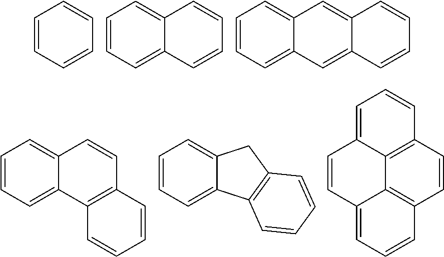

Examples of the alicyclic structure as a basic skeleton include:

monocyclic cycloalkane structures such as cyclopropane, cyclobutane, cyclopentane, cyclohexane, and cycloheptane;

polycyclic cycloalkane structures such as norbornane, adamantane, tricyclodecane, tetracyclododecane, and decahydronaphthalene;

monocyclic cycloalkene structure such as cyclopropene, cyclobutene, cyclopentene, and cyclohexene; and

polycyclic cycloalkenyl structures such as norbornene, tricyclodecene, and tetracyclododecene.

Among them, the alicyclic structure is preferably at least one selected from the group consisting of the following structures.

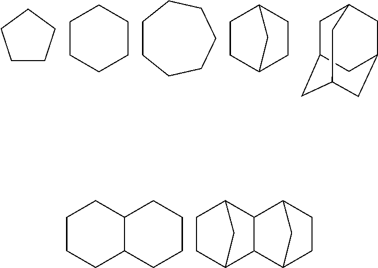

Examples of the heterocyclic structure as a basic skeleton include an aromatic heterocyclic structure and an alicyclic heterocyclic structure in which one or more heteroatoms are interposed between carbon atoms forming the skeleton of the aromatic ring structure or the alicyclic structure. Five-membered aromatic structures which have aromaticity due to introduction of a heteroatom are also included in the heterocyclic structure. Examples of the heteroatom include an oxygen atom, a nitrogen atom, and a sulfur atom.

Examples of the aromatic heterocyclic structure include:

oxygen atom-containing aromatic heterocyclic structures such as furan, pyran, benzofuran, and benzopyran;

nitrogen atom-containing aromatic heterocyclic structures such as pyrrole, imidazole, pyridine, pyrimidine, pyrazine, indole, quinoline, isoquinoline, acridine, phenazine, and carbazole;

sulfur atom-containing aromatic heterocyclic structure such as thiophene; and

aromatic heterocyclic structures containing a plurality of heteroatoms, such as thiazole, benzothiazole, thiazine, and oxazine.

Examples of the alicyclic heterocyclic structure include:

oxygen atom-containing alicyclic heterocyclic structures such as oxirane, tetrahydrofuran, tetrahydropyran, dioxolane, and dioxane;

nitrogen atom-containing alicyclic heterocyclic structures such as aziridine, pyrrolidine, piperidine, and piperazine;

sulfur atom-containing alicyclic heterocyclic structures such as thietane, thiolane, and thiane; and

alicyclic heterocyclic structures containing a plurality of heteroatoms, such as morpholine, 1,2-oxathiolane, and 1,3-oxathiolane.

Examples of the lactone structure, cyclic carbonate structure, and sultone structure include structures represented by the following formulas (H-1) to (H-9).

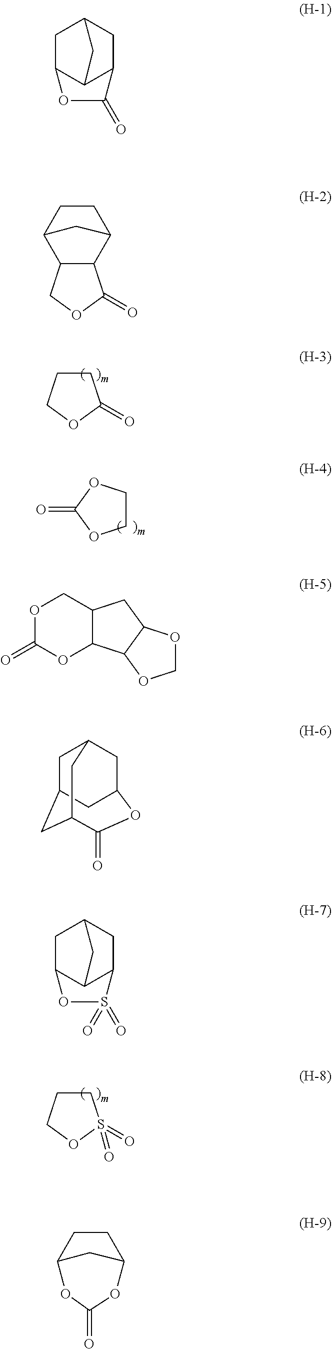

In the above formulas, m is an integer of 1 to 3.

Among them, the heterocyclic structure is preferably at least one selected from the group consisting of the following structures.

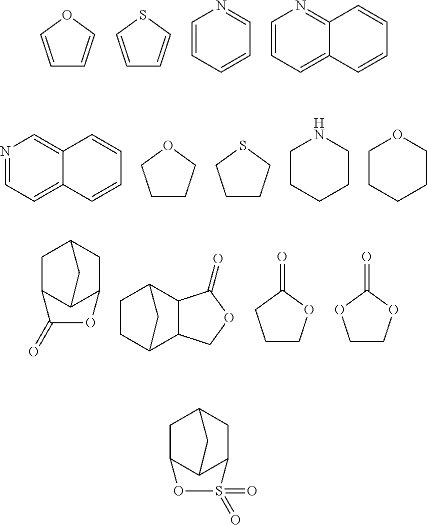

The chain structure when the compound (1) has a plurality of the cyclic structures and the plurality of the cyclic structures are linked via a chain structure is not particularly limited, but is preferably a single bond, —O—, —COO—, —OCO—, —CO—, an alkanediyl group, or a combination thereof. Examples of the alkanediyl group include alkanediyl groups having 1 to 8 carbon atoms, such as a methanediyl group, an ethanediyl group, a propanediyl group, and a butanediyl group. As a result, the molecular design can be appropriately adjusted according to the characteristics required for the compound (1).

Examples of the substituent that substitutes for the hydrogen atom on the carbon atom of the cyclic structure or the chain structure include halogen atoms such as a fluorine atom, a chlorine atom, a bromine atom, and an iodine atom; a hydroxy group; a carboxy group; a cyano group; a nitro group; an alkyl group, an alkoxy group, an alkoxycarbonyl group, an alkoxycarbonyloxy group, an acyl group, an acyloxy group, or a group in which a hydrogen atom of these groups has been substituted with a halogen atom; an oxo group (═O). These substituents may include a cyclic structure.

The substituted or unsubstituted monovalent organic group having a cyclic structure in the above formula (1) has one of the above cyclic structures or two or more of the above cyclic structures in combination, and has the above chain structure in combination with the cyclic structure as necessary. The organic group may have a plurality of types of cyclic structures, or may have a plurality of cyclic structures of the same type. Similarly, the chain structure may have a plurality of types of chain structures, or may have a plurality of chain structures of the same type.

In the above formula (1), the chain hydrocarbon group having 2 or more carbon atoms is not particularly limited, and examples thereof include chain hydrocarbon groups having 2 to 20 carbon atoms. Examples of the chain hydrocarbon group having 2 to 20 carbon atoms include linear or branched saturated hydrocarbon groups having 2 to 20 carbon atoms and linear or branched unsaturated hydrocarbon groups having 2 to 20 carbon atoms. The number of carbon atoms may be 2 or more, but the lower limit thereof is preferably 3, more preferably 4, still more preferably 5, particularly preferably 6. The upper limit of the number of carbon atoms is preferably 18, more preferably 16, still more preferably 14, particularly preferably 12. The chain hydrocarbon group having 2 to 20 carbon atoms is preferably a chain saturated hydrocarbon group having 2 to 20 carbon atoms.

A part or all of the hydrogen atoms on the carbon atoms of the chain hydrocarbon group may be substituted with a halogen atom such as a fluorine atom, a chlorine atom, a bromine atom, and an iodine atom.

The linear saturated hydrocarbon group having 2 to 20 carbon atoms is preferably at least one selected from the group consisting of the following structures.

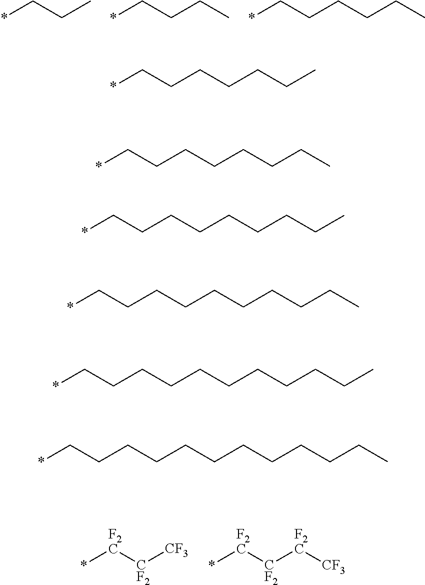

(wherein * is a linking part with another structure)

Preferable examples of the branched saturated hydrocarbon group having 2 to 20 carbon atoms include at least one selected from the group consisting of the following structures.

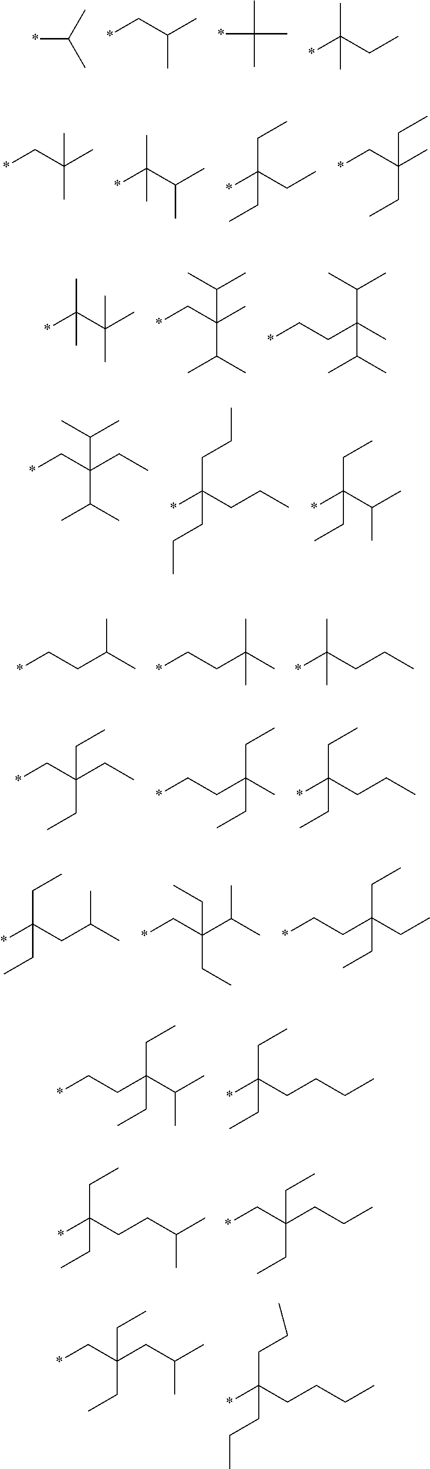

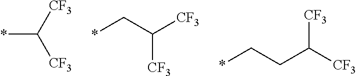 [SMILES: *C(C(F)(F)F)C(F)(F)F ; *CC(C(F)(F)F)C(F)(F)F ; *CCC(C(F)(F)F)C(F)(F)F]

(wherein is a linking part with another structure)

The anion moiety in the compound (1) may have any structure obtained by combining the cyclic structure and, as necessary, a chain structure, or the chain hydrocarbon group. Specific examples of the anion moiety are not particularly limited, and include, for example, structures represented by the following formula.

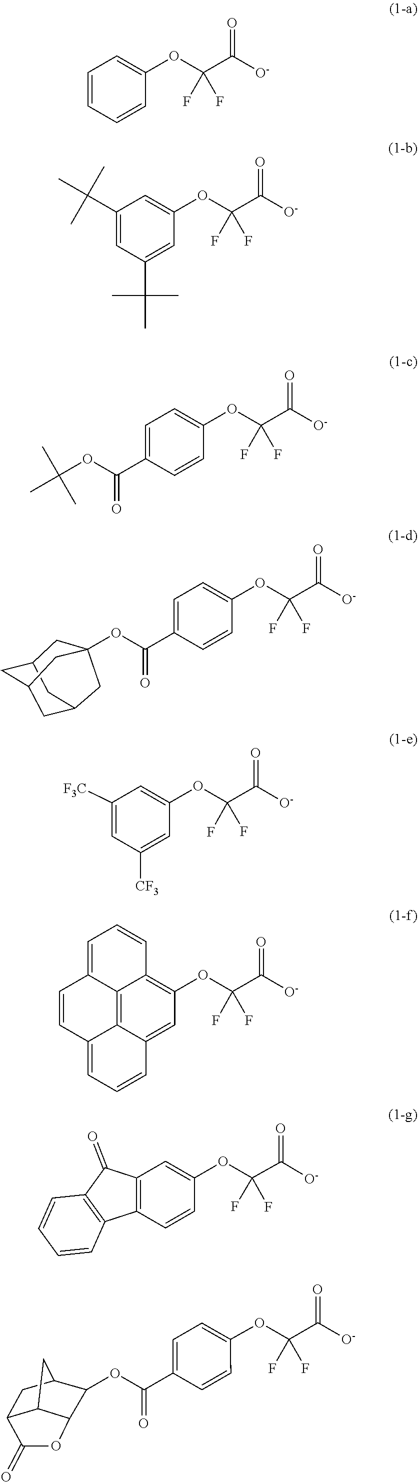

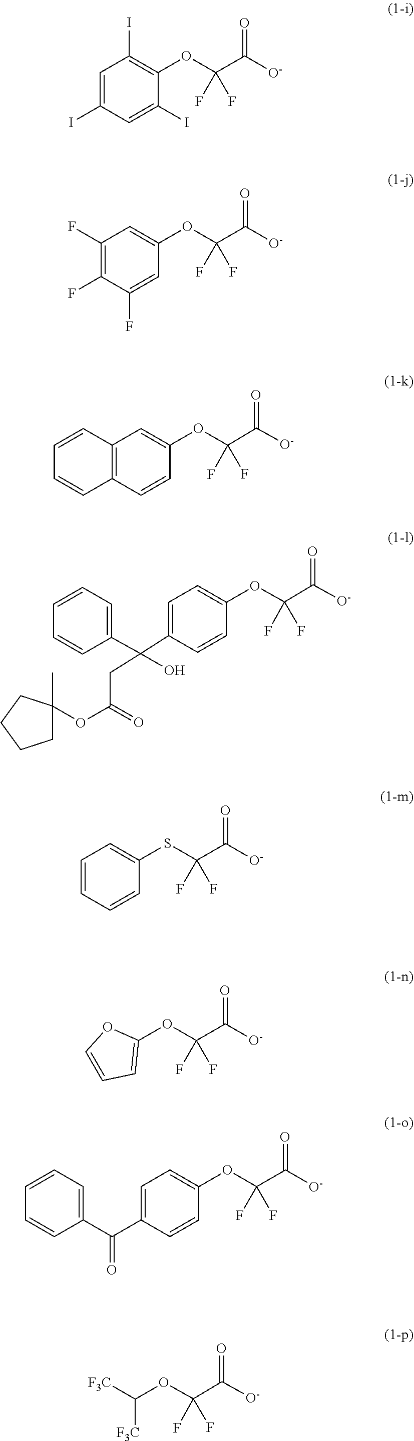

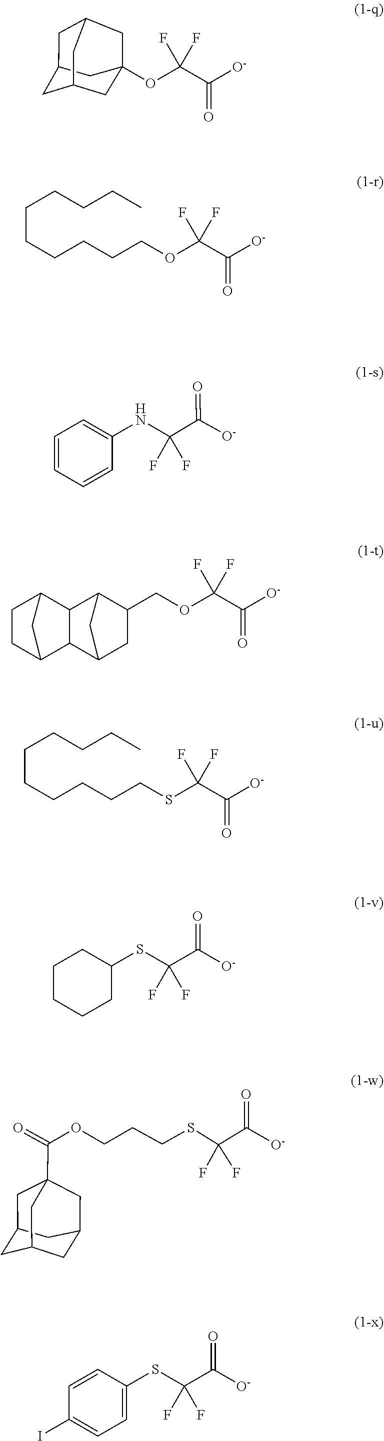

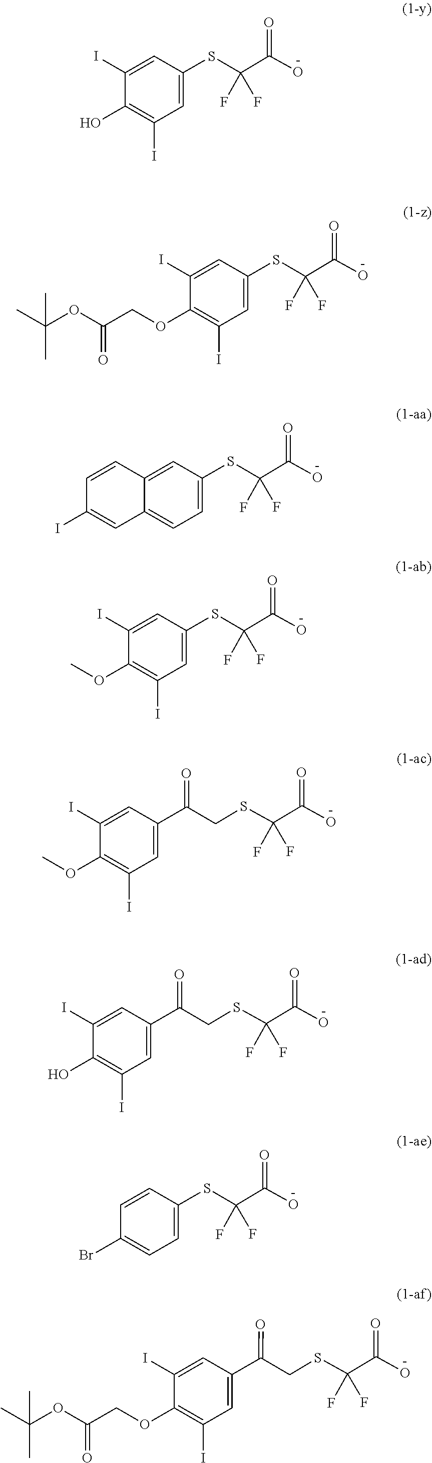

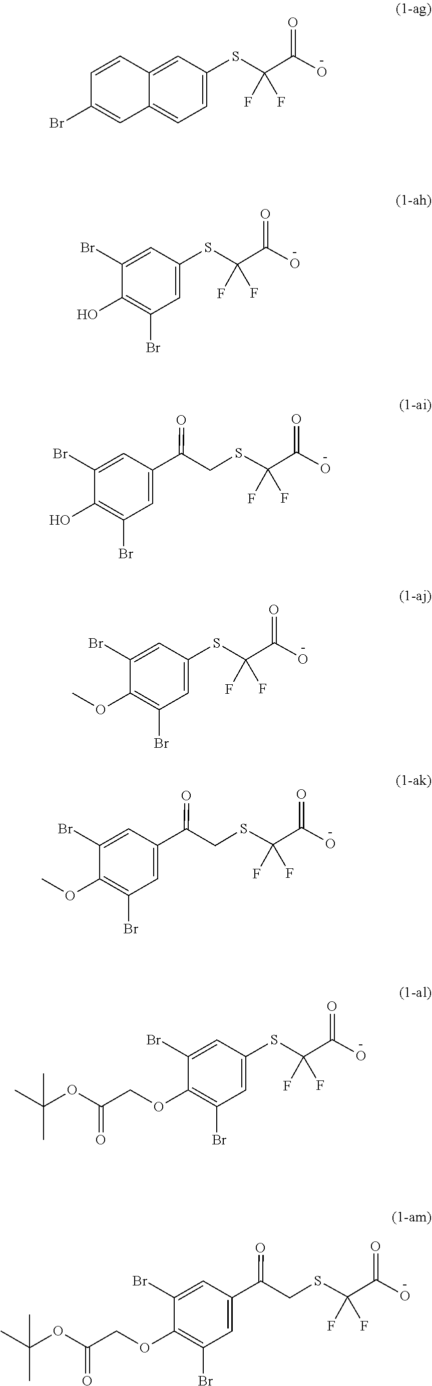

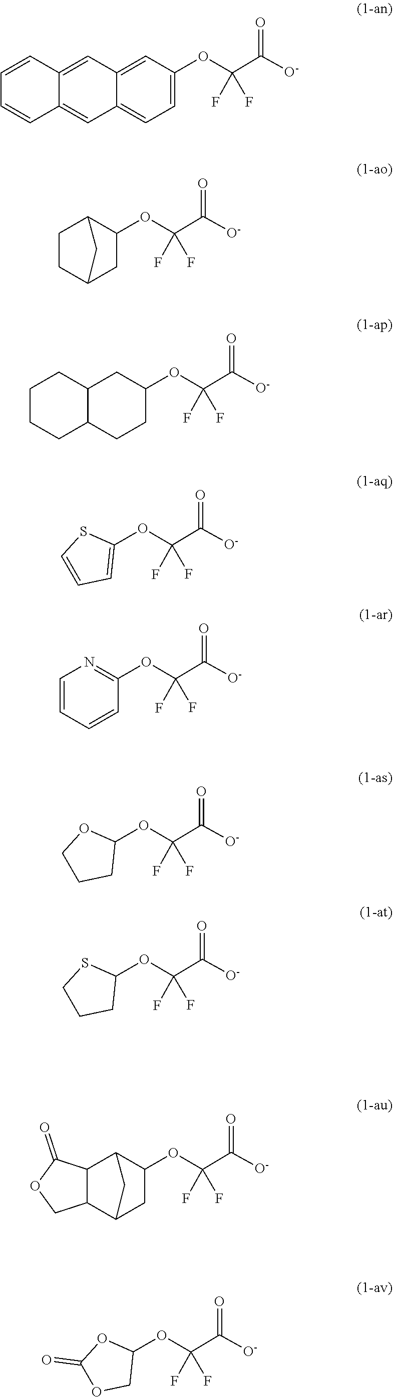

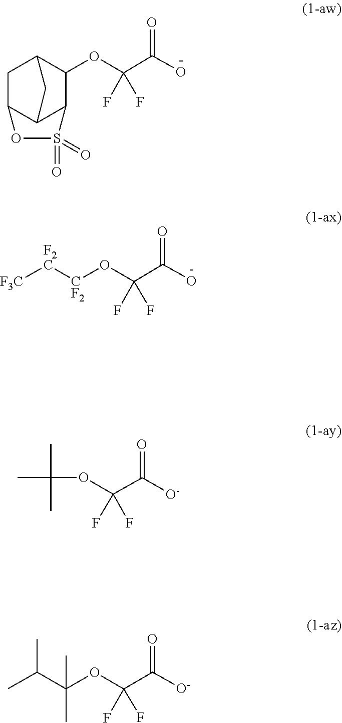 [SMILES: O=C(O)C(F)(F)OC1C2CC3OS(=O)(=O)C1C3C2 ; O=C(O)C(F)(F)OC(F)(F)C(F)(F)C(F)(F)F ; CC(C)(C)OC(F)(F)C(=O)[O-] ; CC(C)C(C)(C)OC(F)(F)C(=O)[O-]]

An example of the monovalent onium cation is a radioactive ray-degradable onium cation containing an element such as S, I, O, N, P, Cl, Br, F, As, Se, Sn, Sb, Te, or Bi. Examples of such a radioactive ray-degradable onium cation include a sulfonium cation, a tetrahydrothiophenium cation, a iodonium cation, a phosphonium cation, a diazonium cation, and a pyridinium cation. Among them, a sulfonium cation or a iodonium cation is preferred. The sulfonium cation or the iodonium cation is preferably represented by any of the following formulas (X-1) to (X-6).

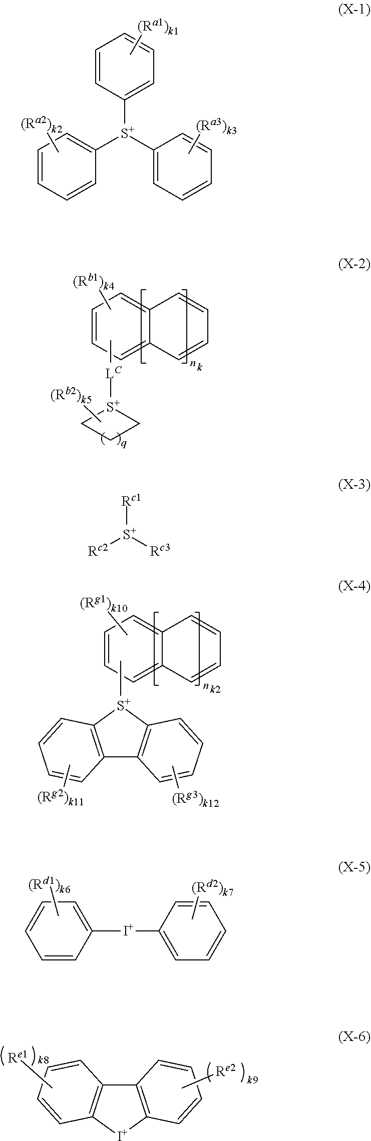

In the above formula (X-1), R^{a1}, R^{Ra2} and R^{a3} are each independently a substituted or unsubstituted, straight or branched chain alkyl group, alkoxy group or alkoxycarbonyloxy group having a carbon number of 1 to 12; a substituted or unsubstituted, monocyclic or polycyclic cycloalkyl group having a carbon number of 3 to 12; a substituted or unsubstituted aromatic hydrocarbon group having a carbon number of 6 to 12; a hydroxy group, a halogen atom, —OSO₂—R^{P}, —SO₂—R^{Q} or —S—R^{T}; or a ring structure obtained by combining two or more of these groups. The ring structure may contain heteroatoms such as O and S between the carbon-carbon bonds forming the skeleton. R^{P}, R^{Q} and R^{T} are each independently a substituted or unsubstituted, straight or branched chain alkyl group having a carbon number of 1 to 12; a substituted or unsubstituted alicyclic hydrocarbon group having a carbon number of 5 to 25; and a substituted or unsubstituted aromatic hydrocarbon group having a carbon number of 6 to 12. k1, k2 and k3 are each independently an integer of 0 to 5. When there are a plurality of R^{a1} to R^{a3} and a plurality of R^{P}, R^{Q} and R^{T}, a plurality of R^{a1} to R^{a3} and a plurality of R^{P}, R^{Q} and R^{T} may be each identical or different.

In the above formula (X-2), R^{b1} is a substituted or unsubstituted, straight chain or branched alkyl group or alkoxy group having a carbon number of 1 to 20; a substituted or unsubstituted acyl group having a carbon number of 2 to 8; or a substituted or unsubstituted aromatic hydrocarbon group having a carbon number of 6 to 8; or a hydroxy group. nₖ is 0 or 1. When nₖ is 0, k4 is an integer of 0 to 4. When nₖ is 1, k4 is an integer of 0 to 7. When there are a plurality of R^{b1}, a plurality of R^{b1} may be each identical or different. A plurality of R^{b1} may represent a ring structure obtained by combining them. R^{b2} is a substituted or unsubstituted, straight chain or branched alkyl group having a carbon number of 1 to 7; or a substituted or unsubstituted aromatic hydrocarbon group having a carbon number of 6 or 7. L^{C} is a single bond or divalent linking group. k5 is an integer of 0 to 4. When there are a plurality of R^{b2}, a plurality of R^{b2} may be each identical or different. A plurality of R^{b2} may represent a ring structure obtained by combining them. q is an integer of 0 to 3. In the formula, the ring structure containing S⁺ may contain a heteroatom such as 0 or S between the carbon-carbon bonds forming the skeleton.

In the above formula (X-3), R^{c1}, R^{c2} and R^{c3} are each independently a substituted or unsubstituted, straight or branched chain alkyl group having a carbon number of 1 to 12.

In the above formula (X-4), R^{g1} is a substituted or unsubstituted linear or branched alkyl or alkoxy group having 1 to 20 carbon atoms, a substituted or unsubstituted acyl group having 2 to 8 carbon atoms, a substituted or unsubstituted aromatic hydrocarbon group having 6 to 8 carbon atoms, or a hydroxy group. nₖ is 0 or 1. When nₖ₂ is 0, k10 is an integer of 0 to 4, and when nₖ₂ is 1, k10 is an integer of 0 to 7. When there are two or more R^{g1}s, the two or more R^{g1}s are the same or different from each other, and may represent a cyclic structure formed by combining them together. R^{g2} and R^{g3} are each independently a substituted or unsubstituted linear or branched alkyl, alkoxy, or alkoxycarbonyloxy group having 1 to 12 carbon atoms, a substituted or unsubstituted monocyclic or polycyclic cycloalkyl group having 3 to 12 carbon atoms, a substituted or unsubstituted aromatic hydrocarbon group having 6 to 12 carbon atoms, a hydroxyl group, a halogen atom, or a ring structure formed by combining two or more of these groups together. K11 and k12 are each independently an integer of 0 to 4. When there are two or more R^{g2}s and two or more R^{g3}s, the two or more R^{g2}s may be the same or different from each other, and the two or more R^{g3}s may be the same or different from each other.

In the above formula (X-5), R^{d1} and R^{d2} are each independently a substituted or unsubstituted, straight or branched chain alkyl group, alkoxy group or alkoxycarbonyl group having a carbon number of 1 to 12; a substituted or unsubstituted aromatic hydrocarbon group having a carbon number of 6 to 12; a halogen atom; a halogenated alkyl group having a carbon number of 1 to 4; a nitro group; or a ring structure obtained by combining two or more of these groups. k6 and k7 are each independently an integer of 0 to 5. When there are a plurality of R^{d1} and a plurality of R^{d2}, a plurality of R^{d1} and a plurality of R^{d2} may be each identical or different.

In the above formula (X-6), R^{e1} and R^{e2} are each independently a halogen atom; a substituted or unsubstituted straight or branched chain alkyl group having a carbon number of 1 to 12; or a substituted or unsubstituted aromatic hydrocarbon group having a carbon number of 6 to 12. k8 and k9 are each independently an integer of 0 to 4.

The compound (1) is formed of a combination of the optional anion moiety containing a monovalent organic group having a cyclic structure or a chain hydrocarbon group and the monovalent onium cation. Specific examples of the compound (1) suitably include the following formulas (1-1) to (1-52).

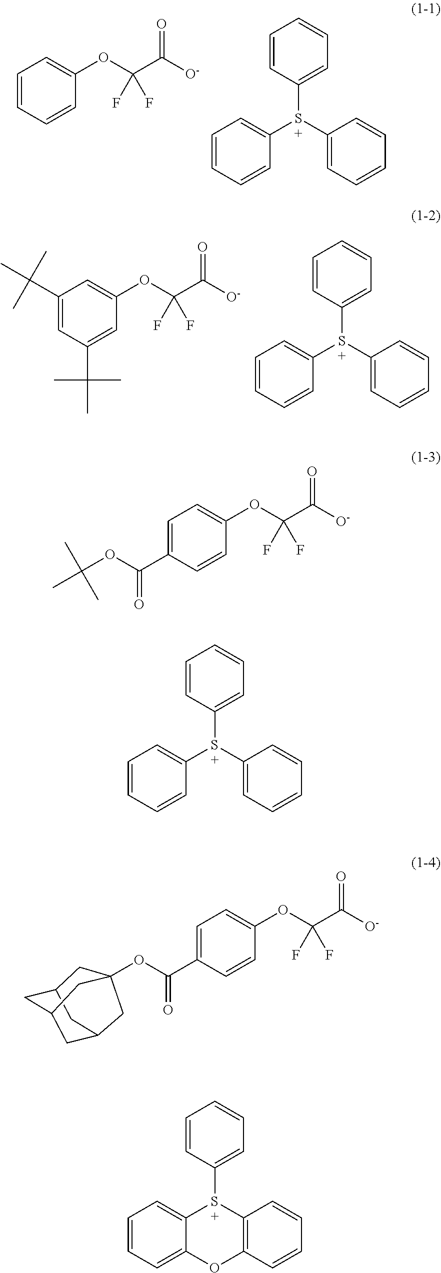

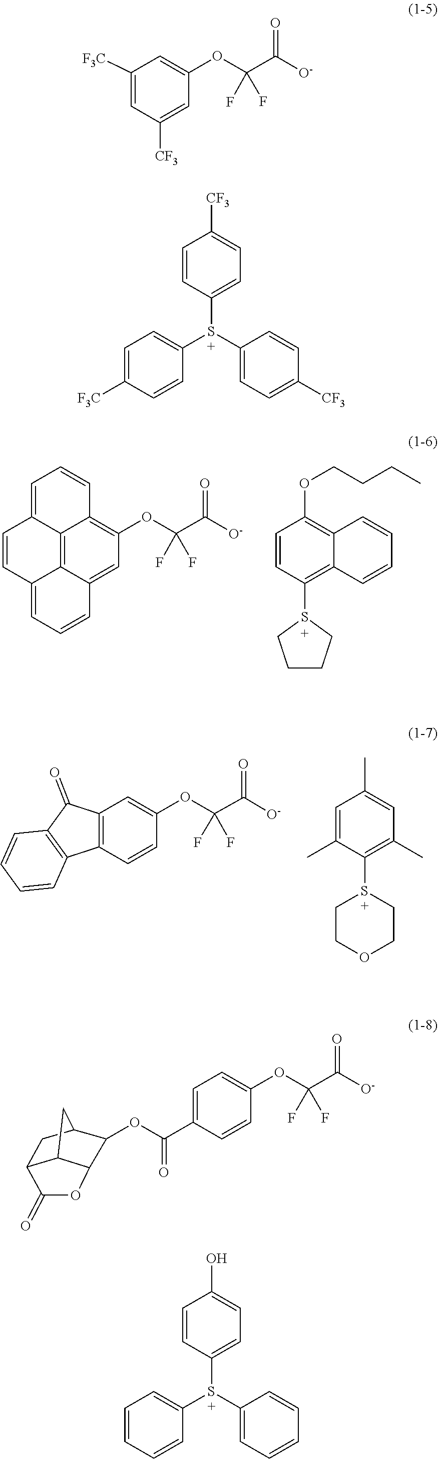

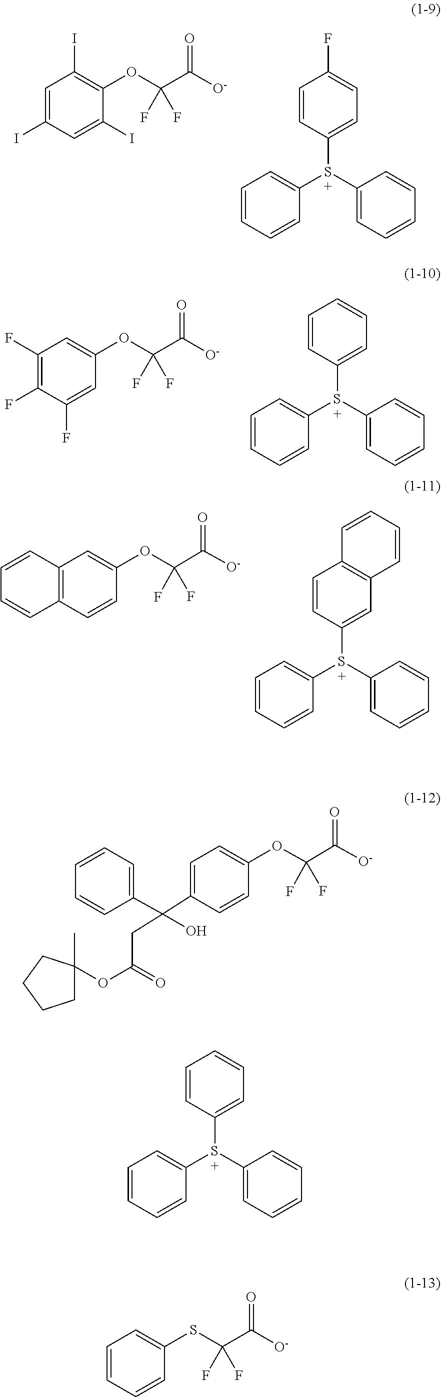

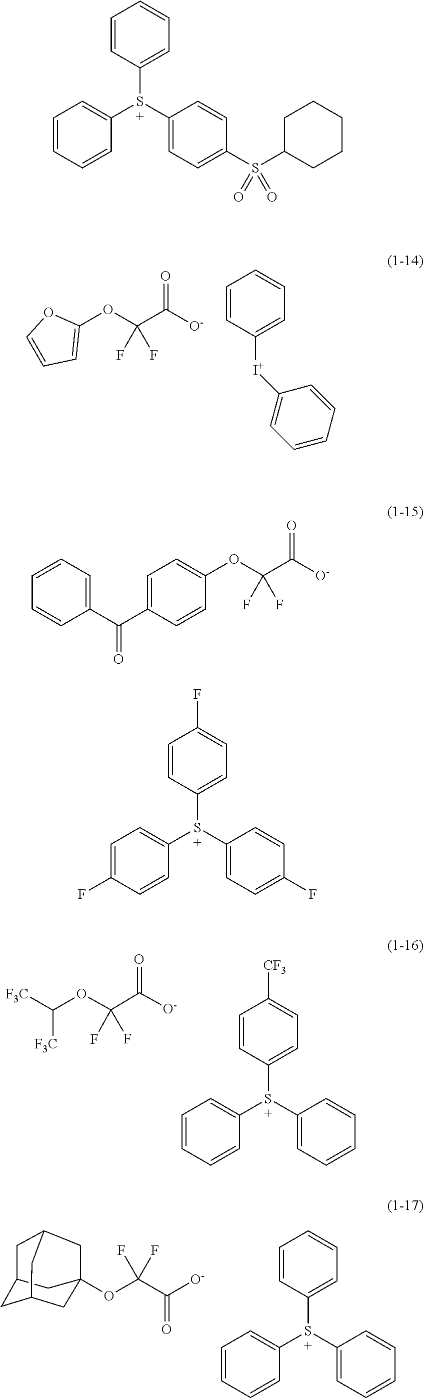

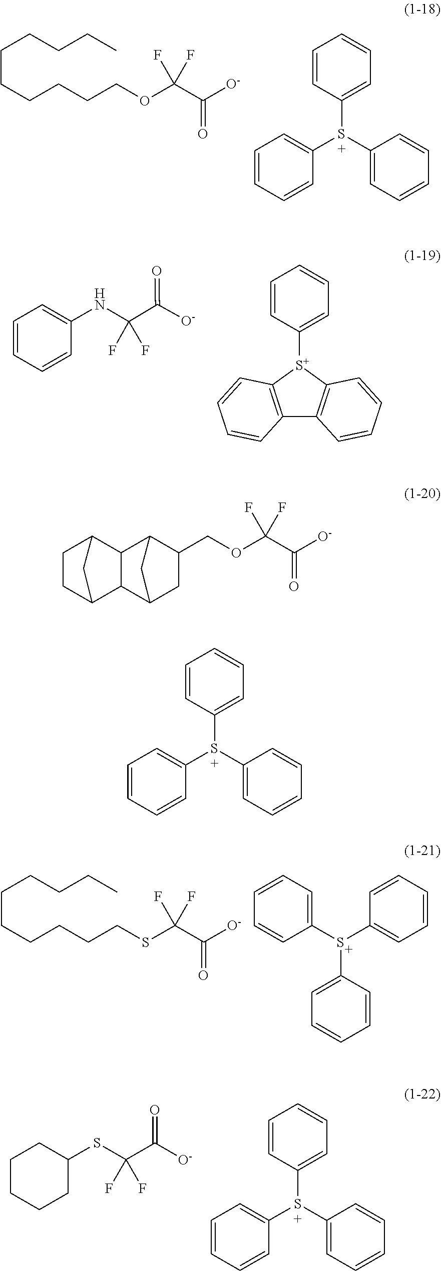

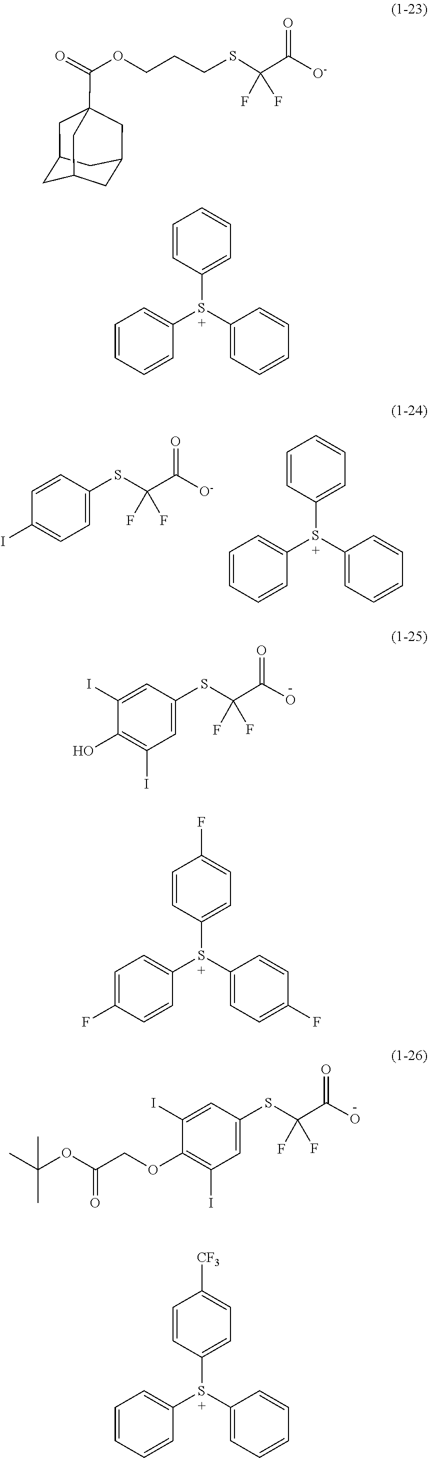

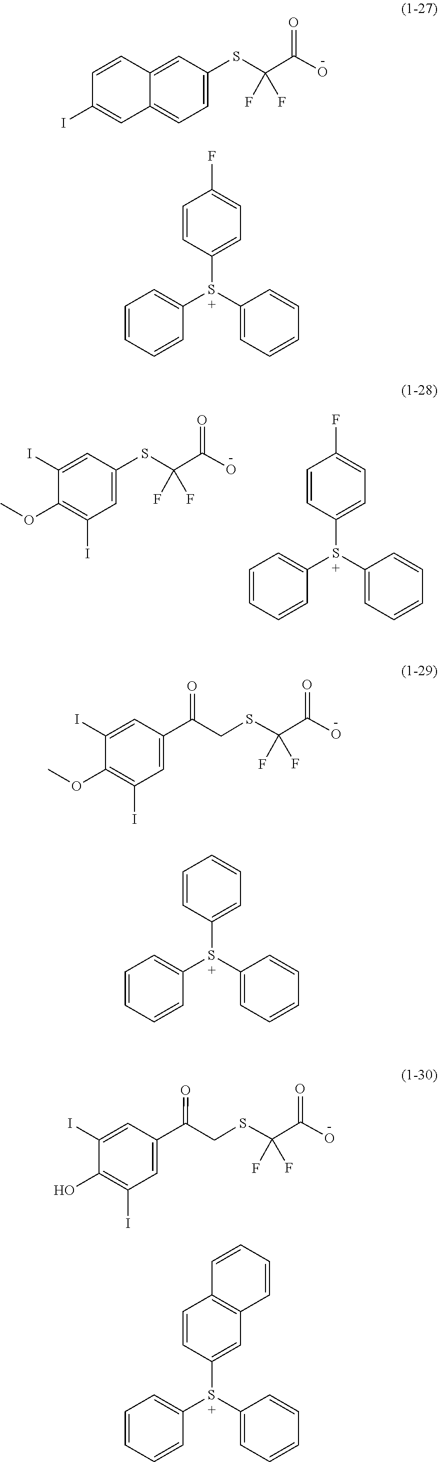

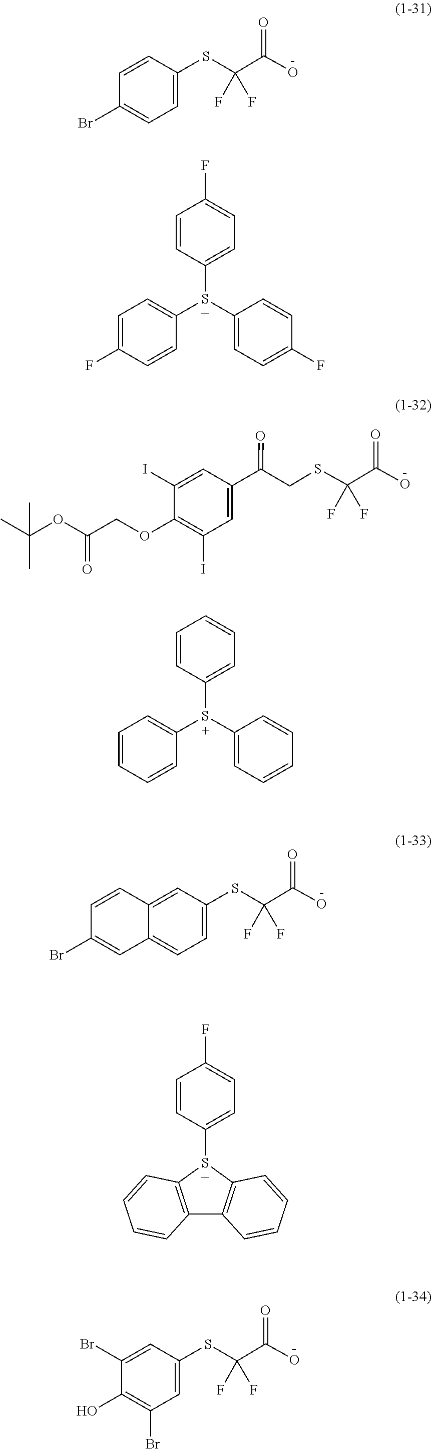

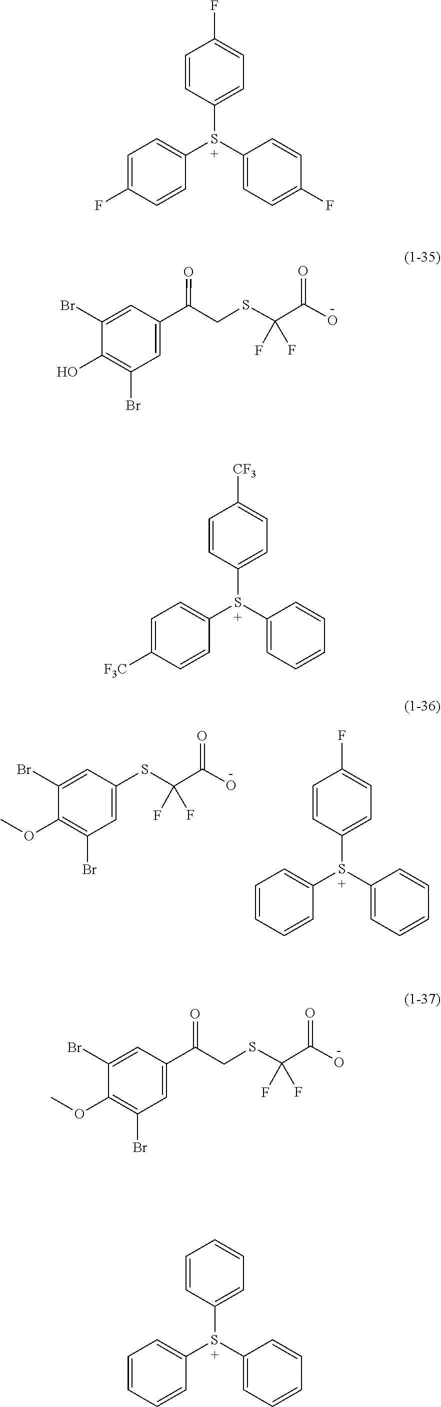

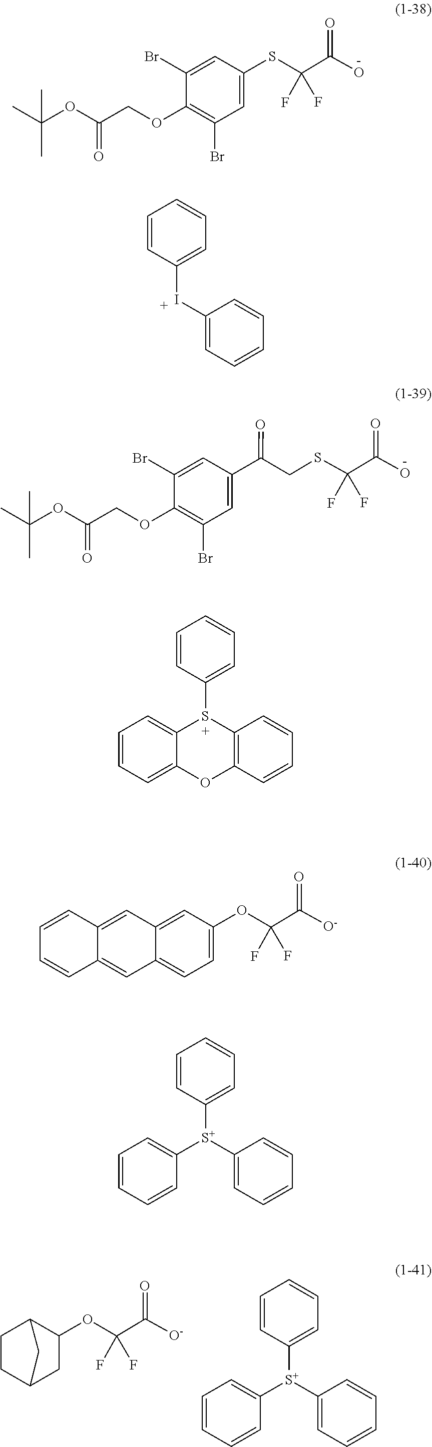

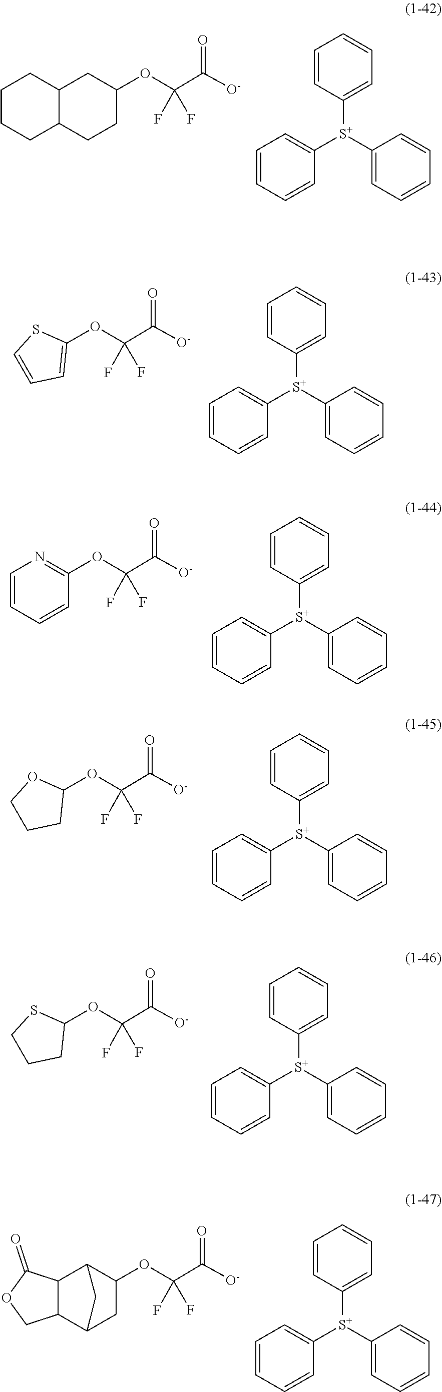

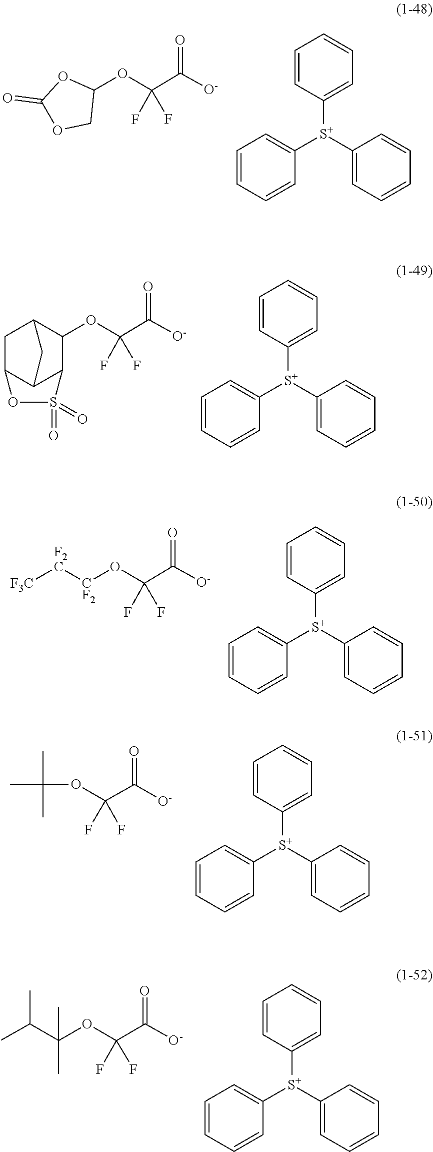

Among them, the compounds (1) represented by the above formulas (1-1) to (1-39) are preferable.

The content of the compound (1) in the radiation-sensitive resin composition according to the present embodiment is preferably 0.1 parts by mass or more and 20 parts by mass or less with respect to 100 parts by mass of the resin described later. The upper limit of the content is more preferably 18 parts by mass, still more preferably 15 parts by mass, particularly preferably 10 parts by mass. The lower limit of the content is more preferably 1 part by mass, still more preferably 2 parts by mass. The content of the compound (1) is appropriately selected according to the type of resin to be used, exposure conditions, required sensitivity, and the type and content of the radiation-sensitive acid generator described later. This makes it possible to exhibit excellent sensitivity, CDU performance, and LWR performance in the formation of the resist pattern.

### (Synthesis Method of Compound (1))

The compound (1) can be typically synthesized according to the following scheme.

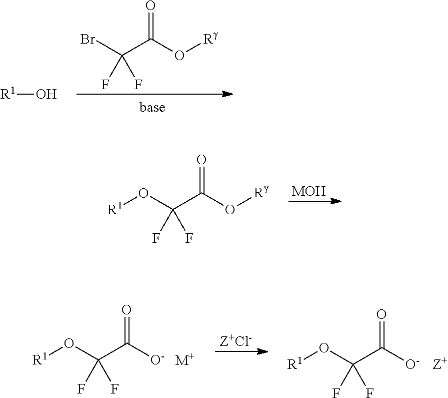

(wherein R¹ and Z⁺ have the same meaning as in the above formula (1), R^{γ} is a monovalent hydrocarbon group, and M is an alkali metal)

A nucleophilic substitution reaction of an alcohol having a structure corresponding to the monovalent organic group having a cyclic structure or the chain hydrocarbon group in the anion moiety with α-bromodifluorocarboxylate is allowed to proceed under basic conditions, to thereby produce an anion moiety precursor. Subsequently, the anion moiety precursor is hydrolyzed with a metal hydroxide, and finally reacted with an onium cation chloride corresponding to the onium cation moiety to proceed salt exchange, whereby the target compound (1) can be synthesized. Similarly, the compound (1) having another structure can be synthesized by appropriately selecting a precursor corresponding to the anion moiety and the onium cation moiety.

### (Resin)

The resin is an aggregate of polymers having a structural unit (hereinafter, also referred to as “structural unit (I)”) containing an acid-dissociable group (hereinafter, this resin is also referred to as “base resin”). The “acid-dissociable group” refers to a group that substitutes for a hydrogen atom of a carboxy group, a phenolic hydroxyl group, an alcoholic hydroxyl group, a sulfo group, or the like, and is dissociated by the action of an acid. The radiation-sensitive resin composition is excellent in pattern-forming performance because the resin has the structural unit (I).

In addition to the structural unit (I), the base resin preferably has a structural unit (II) containing at least one selected from the group consisting of a lactone structure, a cyclic carbonate structure, and a sultone structure described later, and may have another structural unit other than the structural units (I) and (II). Each of the structural units will be described below.

### (Structural Unit (I))

The structural unit (I) contains an acid-dissociable group. The structural unit (I) is not particularly limited as long as it contains an acid-dissociable group. Examples of such a structural unit (I) include a structural unit having a tertiary alkyl ester moiety, a structural unit having a structure obtained by substituting the hydrogen atom of a phenolic hydroxyl group with a tertiary alkyl group, and a structural unit having an acetal bond. From the viewpoint of improving the pattern-forming performance of the radiation-sensitive resin composition, a structural unit represented by the following formula (2) (hereinafter also referred to as a “structural unit (I-1)”) is preferred.

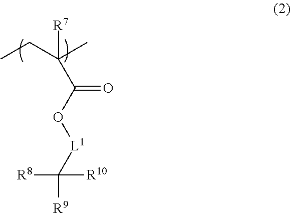

(In the above formula (2), R⁷ is a hydrogen atom, a fluorine atom, a methyl group, or a trifluoromethyl group, R⁸ is a hydrogen atom or a monovalent hydrocarbon group having 1 to 20 carbon atoms, R⁹ and R¹⁰ are each independently a monovalent chain hydrocarbon group having 1 to 10 carbon atoms or a monovalent alicyclic hydrocarbon group having 3 to 20 carbon atoms or represent a divalent alicyclic group having 3 to 20 carbon atoms formed by these groups combined together and a carbon atom to which they are bonded, and L¹ is a single bond or a divalent linking group, and when L¹ is a divalent linking group, a carbon atom bonded to an oxygen atom of —COO— in the above formula (2) is a tertiary carbon or a side chain end-side structure is —COO—).

From the viewpoint of copolymerizability of a monomer that will give the structural unit (I-1), R⁷ is preferably a hydrogen atom or a methyl group, more preferably a methyl group.

Examples of the monovalent hydrocarbon group having 1 to 20 carbon atoms represented by R⁸ include a chain hydrocarbon group having 1 to 10 carbon atoms, a monovalent alicyclic hydrocarbon group having 3 to 20 carbon atoms, and a monovalent aromatic hydrocarbon group having 6 to 20 carbon atoms.

Examples of the chain hydrocarbon groups having 1 to 10 carbon atoms represented by R⁸ to R¹⁰ include linear or branched saturated hydrocarbon groups having 1 to 10 carbon atoms and linear or branched unsaturated hydrocarbon groups having 1 to 10 carbon atoms.

Examples of the alicyclic hydrocarbon groups having 3 to 20 carbon atoms represented by R⁸ to R¹⁰ include monocyclic or polycyclic saturated hydrocarbon groups and monocyclic or polycyclic unsaturated hydrocarbon groups. Preferred examples of the monocyclic saturated hydrocarbon groups include a cyclopentyl group, a cyclohexyl group, a cycloheptyl group, and a cyclooctyl group. Preferred examples of the polycyclic saturated hydrocarbon groups include bridged alicyclic hydrocarbon groups such as a norbornyl group, an adamantyl group, a tricyclodecyl group, and a tetracyclododecyl group. It is to be noted that the bridged alicyclic hydrocarbon group refers to a polycyclic alicyclic hydrocarbon group in which two carbon atoms that constitute an alicyclic ring and not adjacent to each other are bonded by a bonding chain containing at least one carbon atom.

Examples of the monovalent aromatic hydrocarbon group having 6 to 20 carbon atoms represented by R⁸ include: aryl groups such as a phenyl group, a tolyl group, a xylyl group, a naphthyl group, and an anthryl group; and aralkyl groups such as a benzyl group, a phenethyl group, and a naphthylmethyl group.

R⁸ is preferably a linear or branched saturated hydrocarbon group having 1 to 10 carbon atoms or an alicyclic hydrocarbon group having 3 to 20 carbon atoms.

The divalent alicyclic group having 3 to 20 carbon atoms formed by R⁹ and R¹⁰ combined together and a carbon atom to which a chain hydrocarbon group or an alicyclic hydrocarbon group represented by R⁹ and a chain hydrocarbon group or an alicyclic hydrocarbon group represented by R¹⁰ are bonded is not particularly limited as long as it is a group obtained by removing two hydrogen atoms from the same carbon atom constituting a carbon ring of a monocyclic or polycyclic alicyclic hydrocarbon having the above-described carbon number. The divalent alicyclic group having 3 to 20 carbon atoms may either be a monocyclic hydrocarbon group or a polycyclic hydrocarbon group. The polycyclic hydrocarbon group may either be a bridged alicyclic hydrocarbon group or a condensed alicyclic hydrocarbon group and may either be a saturated hydrocarbon group or an unsaturated hydrocarbon group. It is to be noted that the condensed alicyclic hydrocarbon group refers to a polycyclic alicyclic hydrocarbon group in which two or more alicyclic rings share their sides (bond between two adjacent carbon atoms).

When the monocyclic alicyclic hydrocarbon group is a saturated hydrocarbon group, preferred examples thereof include a cyclopentanediyl group, a cyclohexanediyl group, a cycloheptanediyl group, and a cyclooctanediyl group. When the monocyclic alicyclic hydrocarbon group is an unsaturated hydrocarbon group, preferred examples thereof include a cyclopentenediyl group, a cyclohexenediyl group, a cycloheptenediyl group, a cyclooctenediyl group, and a cyclodecenediyl group. The polycyclic alicyclic hydrocarbon group is preferably a bridged alicyclic saturated hydrocarbon group, and preferred examples thereof include a bicyclo[2.2.1]heptane-2,2-diyl group (norbornane-2,2-diyl group), a bicyclo[2.2.2]octane-2,2-diyl group, and a tricyclo[3.3.1.1³′⁷]decane-2,2-diyl group (adamantane-2,2-diyl group).

Examples of the divalent linking group represented by L¹ include an alkanediyl group, a cycloalkanediyl group, an alkenediyl group, *—R^{LA}O—, *—R^{LB}COO— (* represents an oxygen-side bonding hand). However, in the case of the group other than *—R^{LB}COO—, a carbon atom bonded to an oxygen atom of —COO— in the above formula (2) is a tertiary carbon and does not have a hydrogen atom. The tertiary carbon is obtained when the same carbon atom in the group has two bonding hands or when one or two substituents are further bonded to a carbon atom having one of the bonding hands in the group. A part or all of the hydrogen atoms contained in these groups may be substituted with a halogen atom such as a fluorine atom or a chlorine atom, a cyano group or the like.

The alkanediyl group is preferably an alkanediyl group having 1 to 8 carbon atoms.

Examples of the cycloalkanediyl group include: monocyclic cycloalkanediyl groups such as a cyclopentanediyl group and a cyclohexanediyl group; and polycyclic cycloalkanediyl groups such as a norbornanediyl group and an adamantanediyl group. The cycloalkanediyl group is preferably a cycloalkanediyl group having 5 to 12 carbon atoms.

Examples of the alkenediyl group include an ethenediyl group, a propenediyl group, and a butenediyl group. The alkenediyl group is preferably an alkenediyl group having 2 to 6 carbon atoms.

Examples of R^{LA} of *—R^{LA}O— include the above-described alkanediyl group, the above-described cycloalkanediyl group, and the above-described alkenediyl group. Examples of R^{LB} of *—R^{LB}COO— include the above-described alkanediyl group, the above-described cycloalkanediyl group, the above-described alkenediyl group, and an arenediyl group. Examples of the arenediyl group include a phenylene group, a tolylene group, and a naphthylene group. The arenediyl group is preferably an arenediyl group having 6 to 15 carbon atoms.

Among them, R⁸ is preferably an alkyl group having 1 to 4 carbon atoms, and the alicyclic structure formed by R⁹ and R¹⁰ combined together and a carbon atom to which they are bonded is preferably a polycyclic or monocyclic cycloalkane structure. L¹ is preferably a single bond or *—R^{LA}O—. R^{LA} is preferably an alkanediyl group.

Examples of the structural unit (I-1) include structural units represented by the following formulas (3-1) to (3-6) (hereinafter also referred to as “structural units (I-1-1) to (I-1-6)”).

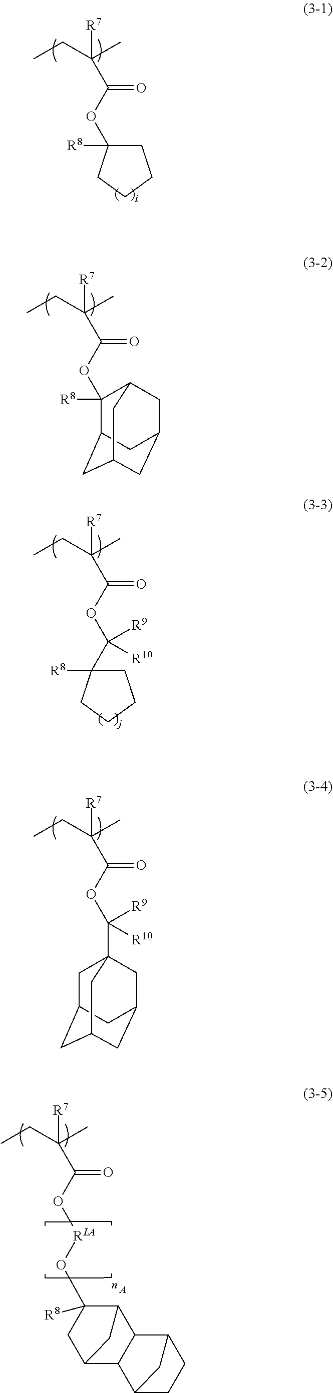

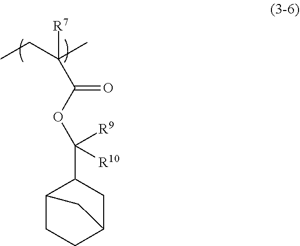

In the above formulas (3-1) to (3-6), R⁷ to R¹⁰ and R^{LA} have the same meaning as in the above formula (2), i and j are each independently an integer of 1 to 4, and n_{A} is 0 or 1.

In the above formulas (3-1) to (3-6), i and j are preferably 1, and R⁸ to R¹⁰ are preferably a methyl group, an ethyl group, or an isopropyl group.

Among them, the structural unit (I-1-1), the structural unit (I-1-2), the structural unit (I-1-4), and the structural unit (I-1-5) are preferable as the structural unit (I-1). The structural unit (I-1-1) preferably has a cyclopentane structure. In the structural unit (I-1-5), n_{A} is preferably 0.

The base resin may contain one type or a combination of two or more types of the structural units (I).

The lower limit of the content of the structural unit (I) is preferably 10 mol %, more preferably 15 mol %, even more preferably 20 mol %, particularly preferably 30 mol % with respect to the total amount of the structural units constituting the base resin. The upper limit of the content is preferably 90 mol %, more preferably 80 mol %, even more preferably 75 mol %, particularly preferably 70 mol %. When the content of the structural unit (I) is set to fall within the above range, the pattern-forming performance of the radiation-sensitive resin composition can further be improved.

### (Structural Unit (II))

The structural unit (II) is a structural unit including at least one selected from the group consisting of a lactone structure, a cyclic carbonate structure and a sultone structure. The solubility of the base resin into a developer can be adjusted by further introducing the structural unit (II). As a result, the radiation-sensitive resin composition can provide improved lithography properties such as the resolution. The adhesion between a resist pattern formed from the base resin and a substrate can also be improved.

Examples of the structural unit (II) include structural units represented by the following formulae (T-1) to (T-10).

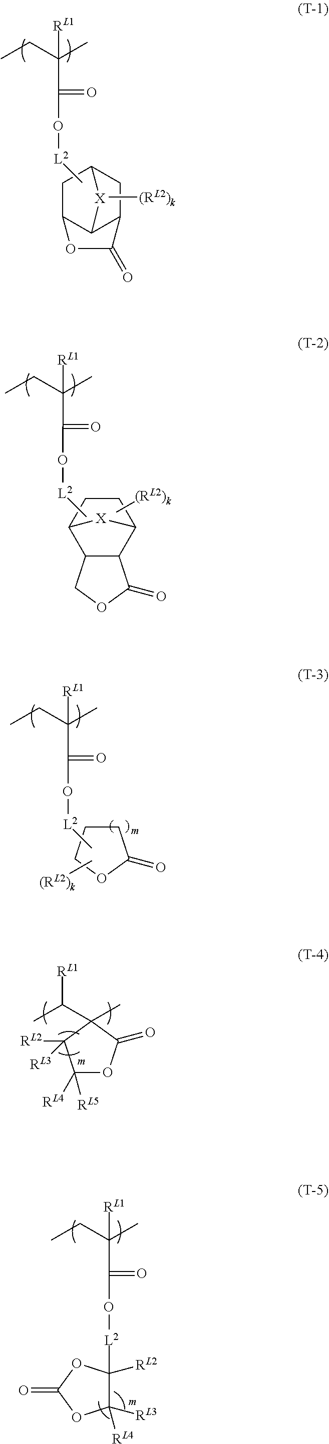

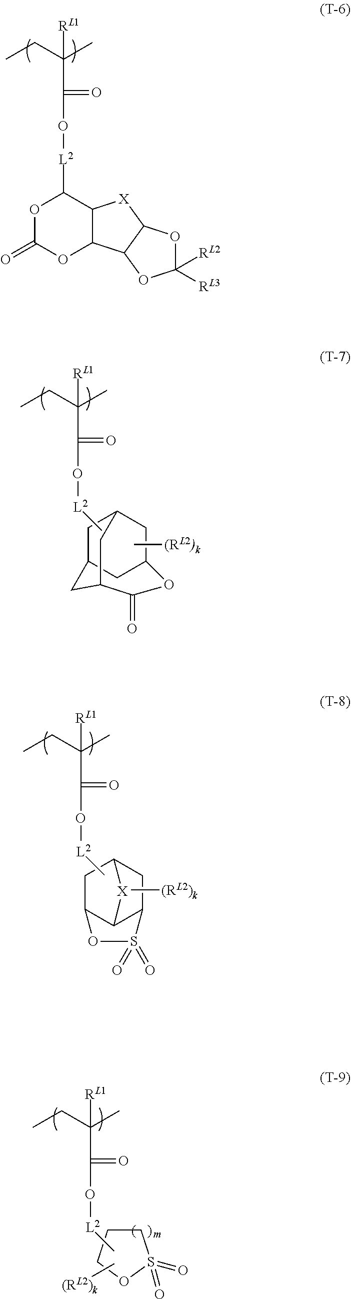

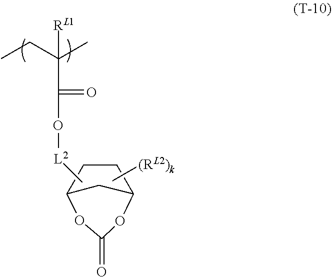

In the above formulae, R^{L1} is a hydrogen atom, a fluorine atom, a methyl group, or a trifluoromethyl group; R^{L2} to R^{L5} are each independently a hydrogen atom, an alkyl group having a carbon number of 1 to 4, a cyano group, a trifluoromethyl group, a methoxy group, a methoxycarbonyl group, a hydroxy group, a hydroxymethyl group, or a dimethylamino group; RL⁴ and R^{L5} may be a divalent alicyclic group having a carbon number of 3 to 8, which is obtained by combining RL⁴ and R^{L5} with the carbon atom to which they are bound. L² is a single bond, or a divalent linking group; X is an oxygen atom or a methylene group; k is an integer of 0 to 3; and m is an integer of 1 to 3.

Example of the divalent alicyclic group having a carbon number of 3 to 8, which is composed of a combination of RL⁴ and R^{L5} with the carbon atom to which they are bound, includes the divalent alicyclic group having a carbon number of 3 to 8 in the divalent alicyclic group having a carbon number of 3 to 20, which is composed of a combination of the chain hydrocarbon group or the alicyclic hydrocarbon group represented by R⁹ and R¹⁰ in the above formula (2) with the carbon atom to which they are bound. One or more hydrogen atoms on the alicyclic group may be substituted with a hydroxy group.

Examples of the divalent linking group represented by L² as described above include a divalent straight or branched chain hydrocarbon group having a carbon number of 1 to 10; a divalent alicyclic hydrocarbon group having a carbon number of 4 to 12; and a group composed of one or more of the hydrocarbon group thereof and at least one group of —CO—, —O—, —NH— and —S—.

Among them, the structural unit (II) is preferably a group having a lactone structure, more preferably a group having a norbornane lactone structure, and further preferably a group derived from a norbornane lactone-yl (meth)acrylate.

The lower limit of the content by percent of the structural unit (II) is preferably 20 mol %, more preferably 25 mol %, and further preferably 30 mol % based on the total structural units as the component of the base resin. The upper limit of the content by percent is preferably 80 mol %, more preferably 70 mol %, and further preferably 60 mol %. By adjusting the content by percent of the structural unit D within the ranges, the radiation-sensitive resin composition can provide improved lithography properties such as the resolution. The adhesion between the formed resist pattern and the substrate can also be improved.

### [Another Structural Unit]

The base resin optionally has another structural unit in addition to the structural units (I) and (II). Examples of the other structural unit include structural units containing a polar group (excluding those corresponding to the structural unit (II)). When the base resin further has a structural unit containing a polar group, solubility in the developer can be adjusted. As a result, lithographic performance such as resolution of the radiation-sensitive resin composition can be improved. Examples of the polar group include a hydroxy group, a carboxy group, a cyano group, a nitro group, and a sulfonamide group. Among them, a hydroxy group and a carboxy group are preferable, and a hydroxy group is more preferable.

Examples of the structural unit including a polar group include structural units represented by the following formulas.

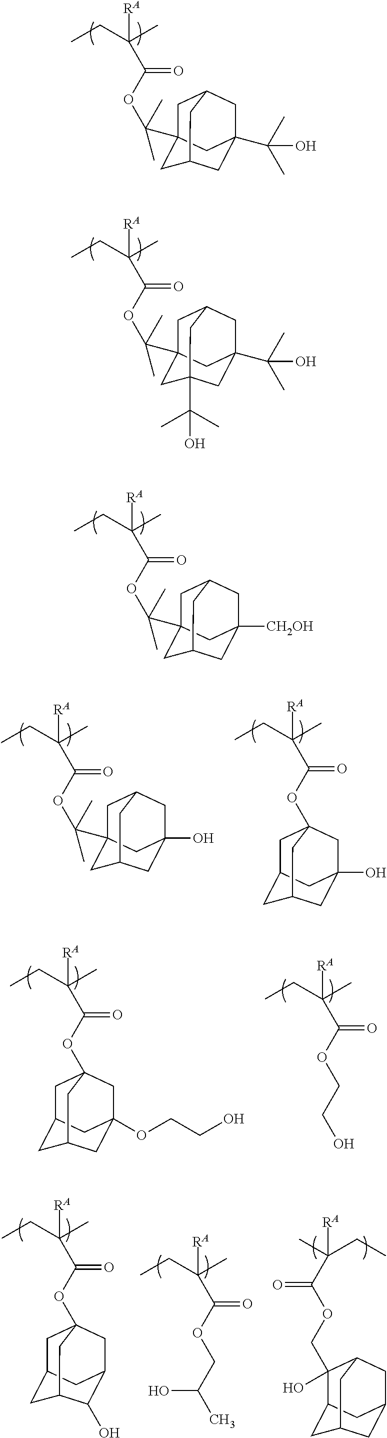

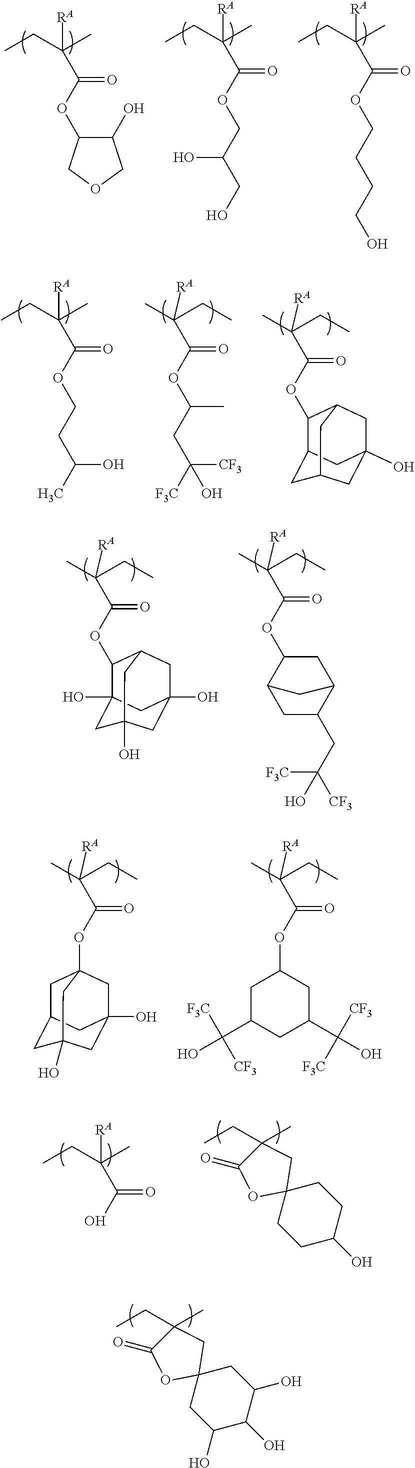

In the above formulas, RA is a hydrogen atom, a fluorine atom, a methyl group, or a trifluoromethyl group.

When the resin includes the structural unit having a polar group, the lower limit of the content of the structural unit having a polar group with respect to the total amount of the structural units constituting the resin is preferably 5 mol %, more preferably 8 mol %, even more preferably 10 mol %. The upper limit of the content is preferably 50 mol %, more preferably 40 mol %, even more preferably 30 mol %. When the content of the structural unit having a polar group is set to fall within the above range, the radiation-sensitive resin composition can provide further improved lithography properties such as the resolution.

The base resin optionally has, as another structural unit, a structural unit derived from hydroxystyrene or a structural unit having a phenolic hydroxyl group (hereinafter, both are also collectively referred to as “structural unit (III)”), in addition to the structural unit having a polar group. The structural unit (III) contributes to an improvement in etching resistance and an improvement in a difference in solubility of a developer (dissolution contrast) between an exposed part and a non-exposed part. In particular, the structural unit (III) can be suitably applied to pattern formation using exposure with a radioactive ray having a wavelength of 50 nm or less, such as an electron beam or EUV. In this case, the resin preferably has the structural unit (I) together with the structural unit (III).

In this case, it is preferable to obtain the structural unit (III) by performing polymerization in a state in which the phenolic hydroxyl group is protected by a protective group such as an alkali-dissociable group during polymerization, and then performing deprotection by hydrolysis. The structural unit that provides the structural unit (III) by hydrolysis is preferably represented by the following formulas (4-1) and (4-2).

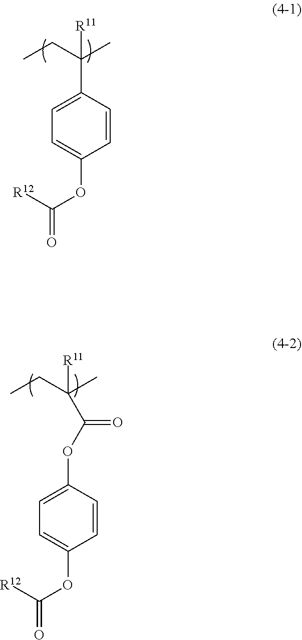

In the above formulas (4-1) and (4-2), R¹¹ is a hydrogen atom, a fluorine atom, a methyl group, or a trifluoromethyl group, and R¹² is a monovalent hydrocarbon group having 1 to 20 carbon atoms or an alkoxy group. Examples of the monovalent hydrocarbon group having 1 to 20 carbon atoms for R¹² include monovalent hydrocarbon groups having 1 to 20 carbon atoms for R⁸ in the structural unit (I). Examples of the alkoxy group include a methoxy group, an ethoxy group, and a tert-butoxy group.

As R¹², an alkyl group and an alkoxy group are preferable, and among them, a methyl group and a tert-butoxy group are more preferable.

In the case of a resin for exposure with a radioactive ray having a wavelength of 50 nm or less, the lower limit of the content of the structural unit (III) is preferably 10 mol %, more preferably 20 mol %, with respect to the total amount of structural units constituting the resin. The upper limit of the content is preferably 70 mol %, more preferably 60 mol %.

### (Synthesis Method of Base Resin)

For example, the base resin can be synthesized by performing a polymerization reaction of each monomer for providing each structural unit with a radical polymerization initiator or the like in a suitable solvent.

Examples of the radical polymerization initiator include an azo-based radical initiator, including azobisisobutyronitrile (AIBN), 2,2′-azobis(4-methoxy-2,4-dimethylvaleronitrile), 2,2′-azobis(2-cyclopropylpropanenitrile), 2,2′-azobis(2,4-dimethylvaleronitrile), and dimethyl 2,2′-azobisisobutyrate; and peroxide-based radical initiator, including benzoyl peroxide, t-butyl hydroperoxide, and cumene hydroperoxide. Among them, AIBN or dimethyl 2,2′-azobisisobutyrate is preferred, and AIBN is more preferred. The radical initiator may be used alone, or two or more radical initiators may be used in combination.

Examples of the solvent used for the polymerization reaction include alkanes including n-pentane, n-hexane, n-heptane, n-octane, n-nonane, and n-decane;

cycloalkanes including cyclohexane, cycloheptane, cyclooctane, decalin, and norbornane;

aromatic hydrocarbons including benzene, toluene, xylene, ethylbenzene, and cumene;

halogenated hydrocarbons including chlorobutanes, bromohexanes, dichloroethanes, hexamethylenedibromide, and chlorobenzenes;

saturated carboxylate esters, including ethyl acetate, n-butyl acetate, i-butyl acetate, and methyl propionate;

ketones including acetone, methyl ethylketone, 4-methyl-2-pentanone, and 2-heptanone;

ethers including tetrahydrofuran, dimethoxyethanes, and diethoxyethanes; and

alcohols including methanol, ethanol, 1-propanol, 2-propanol, and 4-methyl-2-pentanol. The solvent used for the polymerization reaction may be used alone, or two or more solvents may be used in combination.

The reaction temperature of the polymerization reaction is typically from 40° C. to 150° C., and preferably from 50° C. to 120° C. The reaction time is typically from 1 hour to 48 hours, and preferably from 1 hour to 24 hours.

The molecular weight of the base resin is not particularly limited, but the polystyrene-equivalent weight average molecular weight (Mw) measured by gel permeation chromatography (GPC) is preferably 1,000 or more and 50,000 or less, more preferably 2,000 or more and 30,000 or less, still more preferably 3,000 or more and 15,000 or less, particularly preferably 4,000 or more and 12,000 or less. When the Mw of the base resin is less than the lower limit, the heat resistance of the resulting resist film may be deteriorated. When the Mw of the base resin exceeds the above upper limit, the developability of the resist film may be deteriorated.

For the base resin as a base resin, the ratio of Mw to the number average molecular weight (Mn) as determined by GPC relative to standard polystyrene (Mw/Mn) is typically not less than 1 and not more than 5, preferably not less than 1 and not more than 3, and more preferably not less than 1 and not more than 2.

The Mw and Mn of the resin in the specification are amounts measured by using Gel Permeation Chromatography (GPC) with the condition as described below.

GPC column: two G2000HXL, one G3000HXL, and one G4000HXL (all manufactured from Tosoh Corporation)

Column temperature: 40° C.

Eluting solvent: tetrahydrofuran

Flow rate: 1.0 mL/min

Sample concentration: 1.0% by mass

Sample injection amount: 100 μL

Detector: Differential Refractometer

Reference material: monodisperse polystyrene

The content of the base resin is preferably not less than 70% by mass, more preferably not less than 80% by mass, and further preferably not less than 85% by mass based on the total solid content of the radiation-sensitive resin composition.

<Another Resin>

The radiation-sensitive resin composition according to the present embodiment may contain, as another resin, a resin having higher content by mass of fluorine atoms than the above-described base resin (hereinafter, also referred to as a “high fluorine-content resin”). When the radiation-sensitive resin composition contains the high fluorine-content resin, the high fluorine-content resin can be localized in the surface layer of a resist film compared to the base resin, which as a result makes it possible to enhance the water repellency of the surface of the resist film during immersion exposure.

The high fluorine-content resin preferably has, for example, a structural unit represented by the following formula (5) (hereinafter, also referred to as “structural unit (IV)”), and may have the structural unit (I) or the structural unit (II) in the base resin as necessary.

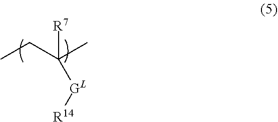

In the above formula (5), R¹³ is a hydrogen atom, a methyl group, or a trifluoromethyl group; G^{L} is a single bond, an oxygen atom, a sulfur atom, —COO—, —SO₂ONH—, —CONH—, or —OCONH—; R¹⁴ is a monovalent fluorinated chain hydrocarbon group having a carbon number of 1 to 20, or a monovalent fluorinated alicyclic hydrocarbon group having a carbon number of 3 to 20.

As R¹³ as described above, in terms of the copolymerizability of monomers resulting in the structural unit (IV), a hydrogen atom or a methyl group is preferred, and a methyl group is more preferred.

As G^{L} as described above, in terms of the copolymerizability of monomers resulting in the structural unit (IV), a single bond or —COO— is preferred, and —COO— is more preferred.

Example of the monovalent fluorinated chain hydrocarbon group having a carbon number of 1 to 20 represented by R¹⁴ as described above includes a group in which a part of or all of hydrogen atoms in the straight or branched chain alkyl group having a carbon number of 1 to 20 is/are substituted with a fluorine atom.

Example of the monovalent fluorinated alicyclic hydrocarbon group having a carbon number of 3 to 20 represented by R¹⁴ as described above includes a group in which a part of or all of hydrogen atoms in the monocyclic or polycyclic hydrocarbon group having a carbon number of 3 to 20 is/are substituted with a fluorine atom.

The R¹⁴ as described above is preferably a fluorinated chain hydrocarbon group, more preferably a fluorinated alkyl group, and further preferably 2,2,2-trifluoroethyl group, 1,1,1,3,3,3-hexafluoropropyl group and 5,5,5-trifluoro-1,1-diethylpentyl group.

When the high fluorine-content resin has the structural unit (IV), the lower limit of the content of the structural unit (IV) is preferably 30 mol %, more preferably 35 mol %, even more preferably 40 mol %, particularly preferably 45 mol % with respect to the total amount of all the structural units constituting the high fluorine-content resin. The upper limit of the content is preferably 90 mol %, more preferably 85 mol %, even more preferably 80 mol %. When the content of the structural unit (IV) is set to fall within the above range, the content by mass of fluorine atoms of the high fluorine-content resin can more appropriately be adjusted to further promote the localization of the high fluorine-content resin in the surface layer of a resist film, as a result, the water repellency of the resist film during immersion exposure can be further improved.

The high fluorine-content resin may have a fluorine atom-containing structural unit represented by the following formula (f-2) (hereinafter, also referred to as a “structural unit (V)”) in addition to or in place of the structural unit (IV). When the high fluorine-content resin has the structural unit (V), solubility in an alkaline developing solution is improved, and therefore generation of development defects can be prevented.

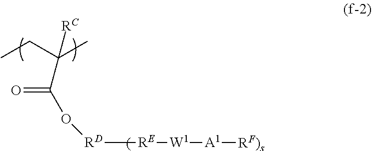

The structural unit (V) is classified into two groups: a unit having an alkali soluble group (x); and a unit having a group (y) in which the solubility into the alkaline developing solution is increased by the dissociation by alkali (hereinafter, simply referred as an “alkali-dissociable group”). In both cases of (x) and (y), R^{C} in the above formula (f-2) is a hydrogen atom, a fluorine atom, a methyl group, or a trifluoromethyl group; R^{D} is a single bond, a hydrocarbon group having a carbon number of 1 to 20 with the valency of (s+1), a structure in which an oxygen atom, a sulfur atom, —NR^{dd}—, a carbonyl group, —COO— or —CONH— is connected to the terminal on R^{E} side of the hydrocarbon group, or a structure in which a part of hydrogen atoms in the hydrocarbon group is substituted with an organic group having a hetero atom; R^{dd} is a hydrogen atom, or a monovalent hydrocarbon group having a carbon number of 1 to 10; and s is an integer of 1 to 3.

When the structural unit (V) has the alkali soluble group (x), R^{F} is a hydrogen atom; A¹ is an oxygen atom, —OCO—* or —SO₂O—*; * refers to a bond to R^{F}; W¹ is a single bond, a hydrocarbon group having a carbon number of 1 to 20, or a divalent fluorinated hydrocarbon group. When A¹ is an oxygen atom, W¹ is a fluorinated hydrocarbon group having a fluorine atom or a fluoroalkyl group on the carbon atom connecting to A₁. R^{E} is a single bond, or a divalent organic group having a carbon number of 1 to 20. When s is 2 or 3, a plurality of R^{E}, W¹, A¹ and R^{F} may be each identical or different. The affinity of the high fluorine-content resin into the alkaline developing solution can be improved by including the structural unit (V) having the alkali soluble group (x), and thereby prevent from generating the development defect. As the structural unit (V) having the alkali soluble group (x), particularly preferred is a structural unit in which A¹ is an oxygen atom and W¹ is a 1,1,1,3,3,3-hexafluoro-2,2-methanediyl group.

When the structural unit (V) has the alkali-dissociable group (y), R^{F} is a monovalent organic group having carbon number of 1 to 30; A¹ is an oxygen atom, —NR^{aa}—, —COO—*, or —SO₂O—*; R^{aa} is a hydrogen atom, or a monovalent hydrocarbon group having a carbon number of 1 to 10; * refers to a bond to R^{F}; W¹ is a single bond, or a divalent fluorinated hydrocarbon group having a carbon number of 1 to 20; R^{E} is a single bond, or a divalent organic group having a carbon number of 1 to 20. When A¹ is —COO—* or —SO₂O—*, W¹ or R^{F} has a fluorine atom on the carbon atom connecting to A¹ or on the carbon atom adjacent to the carbon atom. When A¹ is an oxygen atom, W¹ and R^{E} are a single bond; R^{D} is a structure in which a carbonyl group is connected at the terminal on R^{E} side of the hydrocarbon group having a carbon number of 1 to 20; and R^{F} is an organic group having a fluorine atom. When s is 2 or 3, a plurality of R^{E}, W¹, A¹ and R^{F} may be each identical or different. The surface of the resist film is changed from hydrophobic to hydrophilic in the alkaline developing step by including the structural unit (V) having the alkali-dissociable group (y). As a result, the affinity of the high fluorine-content resin into the alkaline developing solution can be significantly improved, and thereby prevent from generating the development defect more efficiently. As the structural unit (V) having the alkali-dissociable group (y), particularly preferred is a structural unit in which A¹ is —COO—*, and R^{F} or W¹, or both is/are a fluorine atom.

In terms of the copolymerizability of monomers resulting in the structural unit (V), R^{C} is preferably a hydrogen atom or a methyl group, and more preferably a methyl group.

When R^{E} is a divalent organic group, R^{E} is preferably a group having a lactone structure, more preferably a group having a polycyclic lactone structure, and further preferably a group having a norbornane lactone structure.

When the high fluorine-content resin has the structural unit (V), the lower limit of the content of the structural unit (V) is preferably 40 mol %, more preferably 50 mol %, even more preferably 60 mol % with respect to the total amount of all the structural units constituting the high fluorine-content resin. The upper limit of the content is preferably 95 mol %, more preferably 90 mol %, even more preferably 85 mol %. When the content of the structural unit (V) is set to fall within the above range, water repellency of a resist film during immersion exposure can further be improved.

The lower limit of Mw of the high fluorine-content resin is preferably 1,000, more preferably 2,000, further preferably 3,000, and particularly preferably 5,000. The upper limit of Mw is preferably 50,000, more preferably 30,000, further preferably 20,000, and particularly preferably 15,000.

The lower limit of the Mw/Mn of the high fluorine-content resin is typically 1, and more preferably 1.1. The upper limit of the Mw/Mn is typically 5, preferably 3, more preferably 2, and further preferably 1.7.

The lower limit of the content of the high fluorine-content resin is preferably 0.1% by mass, more preferably 0.5% by mass, further preferably 1% by mass, and even further preferably 1.5% by mass based on the total solid content of the radiation-sensitive resin composition. The upper limit of the content is preferably 20% by mass, more preferably 15% by mass, further preferably 10% by mass, and particularly preferably 7% by mass.

The lower limit of the content of the high fluorine-content resin is preferably 0.1 part by mass, more preferably 0.5 part by mass, further preferably 1 part by mass, and particularly preferably 1.5 part by mass based on 100 parts by mass of total base resins. The upper limit of the content is preferably 15 parts by mass, more preferably 10 parts by mass, further preferably 8 parts by mass, and particularly preferably 5 parts by mass.

When the content of the high fluorine-content resin is set to fall within the above range, the high fluorine-content resin can more effectively be localized in the surface layer of a resist film, which as a result makes it possible to further enhance the water repellency of the surface of the resist film during liquid immersion lithography. The radiation-sensitive resin composition may contain one kind of high fluorine-content resin or two or more kinds of high fluorine-content resins.

### (Method for Synthesizing High Fluorine-Content Resin)

The high fluorine-content resin can be synthesized by a method similar to the above-described method for synthesizing a base resin.

### (Radiation-Sensitive Acid Generator)

The radiation-sensitive resin composition of the present embodiment preferably further contains a radiation-sensitive acid generator that generates an acid having a pKa smaller than that of the acid generated from the compound (1) by exposure, that is, a relatively strong acid. When the resin contains the structural unit (I) having an acid-dissociable group, the acid generated from the radiation-sensitive acid generator by exposure can dissociate the acid-dissociable group of the structural unit (I) to generate a carboxy group or the like. This function is different from the function of the compound (1) that suppresses the diffusion of the acid generated from the radiation-sensitive acid generator in the non-exposed part without substantially dissociating the acid-dissociable group or the like of the structural unit (I) or the like of the resin under the pattern formation condition using the radiation-sensitive resin composition. Each function of the compound (1) and the radiation-sensitive acid generator depends on energy required for the dissociation of the acid-dissociable group of the structural unit (I) or the like of the resin, and heat energy conditions applied when a pattern is formed using the radiation-sensitive resin composition, and the like. The containing mode of the radiation-sensitive acid generator in the radiation-sensitive resin composition may be a mode in which the radiation-sensitive acid generator is present alone as a compound (released from a polymer), a mode in which the radiation-sensitive acid generator is incorporated as a part of a polymer, or both of these forms, but a mode in which the radiation-sensitive acid generator is present alone as a compound is preferable.

When the radiation-sensitive resin composition contains the radiation-sensitive acid generator, the polarity of the resin in the exposed part increases, whereby the resin in the exposed part is soluble in the developer in the case of alkaline aqueous solution development, and is poorly soluble in the developer in the case of organic solvent development.

Examples of the radiation-sensitive acid generator include an onium salt compound (excluding the compound (1)), a sulfonimide compound, a halogen-containing compound, and a diazoketone compound. Examples of the onium salt compound include a sulfonium salt, a tetrahydrothiophenium salt, an iodonium salt, a phosphonium salt, a diazonium salt, and a pyridinium salt. Among them, a sulfonium salt and an iodonium salt are preferable.

Examples of the acid generated during exposure include acids that generate sulfonic acid during exposure. Examples of such an acid include a compound in which the carbon atom adjacent to the sulfo group is substituted with one or more fluorine atoms or fluorinated hydrocarbon groups. Among them, as the radiation-sensitive acid generator, a radiation-sensitive acid generator having a cyclic structure is particularly preferable.

These radiation-sensitive acid generators may be used alone or in combination of two or more thereof. The lower limit of the content of the radiation-sensitive acid generator is preferably 0.1 part by mass, more preferably 1 part by mass, and still more preferably 5 parts by mass, with respect to 100 parts by mass of the resin. The upper limit of the content is preferably 40 parts by mass, more preferably 30 parts by mass, and still more preferably 20 parts by mass. As a result, excellent sensitivity, CDU performance, and LWR performance can be exhibited when forming a resist pattern.

<Solvent>

The radiation-sensitive resin composition according to the present embodiment contains a solvent. The solvent is not particularly limited as long as it can dissolve or disperse at least the resin, the radiation-sensitive acid generator, and an additive or the like contained if necessary.

Examples of the solvent include an alcohol-based solvent, an ether-based solvent, a ketone-based solvent, an amide-based solvent, an ester-based solvent, and a hydrocarbon-based solvent.

Examples of the alcohol-based solvent include:

a monoalcohol-based solvent having a carbon number of 1 to 18, including iso-propanol, 4-methyl-2-pentanol, 3-methoxybutanol, n-hexanol, 2-ethylhexanol, furfuryl alcohol, cyclohexanol, 3,3,5-trimethylcyclohexanol, and diacetone alcohol;

a polyhydric alcohol having a carbon number of 2 to 18, including ethylene glycol, 1,2-propylene glycol, 2-methyl-2,4-pentanediol, 2,5-hexanediol, diethylene glycol, dipropylene glycol, triethylene glycol, and tripropylene glycol; and

a partially etherized polyhydric alcohol-based solvent in which a part of hydroxy groups in the polyhydric alcohol-based solvent is etherized.

Examples of the ether-based solvent include:

a dialkyl ether-based solvent, including diethyl ether, dipropyl ether, and dibutyl ether;

a cyclic ether-based solvent, including tetrahydrofuran and tetrahydropyran;

an ether-based solvent having an aromatic ring, including diphenylether and anisole (methyl phenyl ether); and

an etherized polyhydric alcohol-based solvent in which a hydroxy group in the polyhydric alcohol-based solvent is etherized.

Examples of the ketone-based solvent include:

a chain ketone-based solvent, including acetone, butanone, and methyl-iso-butyl ketone;

a cyclic ketone-based solvent, including cyclopentanone, cyclohexanone, and methylcyclohexanone; and 2,4-pentanedione, acetonylacetone, and acetophenone.

Examples of the amide-based solvent include:

a cyclic amide-based solvent, including N,N′-dimethyl imidazolidinone and N-methylpyrrolidone; and

a chain amide-based solvent, including N-methylformamide, N,N-dimethylformamide, N,N-diethylformamide, acetamide, N-methylacetamide, N,N-dimethylacetamide, and N-methylpropionamide.

Examples of the ester-based solvent include:

a monocarboxylate ester-based solvent, including n-butyl acetate and ethyl lactate;

a partially etherized polyhydric alcohol acetate-based solvent, including diethylene glycol mono-n-butyl ether acetate, propylene glycol monomethyl ether acetate, and dipropylene glycol monomethyl ether acetate;

a lactone-based solvent, including γ-butyrolactone and valerolactone;

a carbonate-based solvent, including diethyl carbonate, ethylene carbonate, and propylene carbonate; and

a polyhydric carboxylic acid diester-based solvent, including propylene glycol diacetate, methoxy triglycol acetate, diethyl oxalate, ethyl acetoacetate, ethyl lactate, and diethyl phthalate.

Examples of the hydrocarbon-based solvent include:

an aliphatic hydrocarbon-based solvent, including n-hexane, cyclohexane, and methylcyclohexane;

an aromatic hydrocarbon-based solvent, including benzene, toluene, di-iso-propylbenzene, and n-amylnaphthalene.

Among them, the ester-based solvent or the ketone-based solvent is preferred. The partially etherized polyhydric alcohol acetate-based solvent, the cyclic ketone-based solvent, or the lactone-based solvent is more preferred. Propylene glycol monomethyl ether acetate, cyclohexanone, or γ-butyrolactone is still more preferred. The radiation-sensitive resin composition may include one type of the solvent, or two or more types of the solvents in combination.

<Other Optional Components>

The radiation-sensitive resin composition may contain other optional components other than the above-descried components. Examples of other optional components include a cross-linking agent, a localization enhancing agent, a surfactant, an alicyclic backbone-containing compound, and a sensitizer. These other optional components may be used singly or in combination of two or more of them.

### (Crosslinking Agent)

The crosslinking agent is a compound having two or more functional groups. The crosslinking agent causes a crosslinking reaction in the polymer component (1) by an acid catalytic reaction in the baking step after the collective exposure step to increase the molecular weight of the polymer component (1), and thereby decreases the solubility of the pattern exposed part in the developer. Examples of the functional group include a (meth)acryloyl group, a hydroxymethyl group, an alkoxymethyl group, an epoxy group, and a vinyl ether group.

### (Localization Enhancing Agent)

The localization enhancing agent has an effect of localizing the high fluorine-content resin on the surface of the resist film more effectively. The added amount of the high fluorine-content resin can be decreased compared to the traditionally added amount by including the localization enhancing agent in the radiation-sensitive resin composition. The localization enhancing agent can further prevent from eluting the ingredient of the composition from the resist film to an immersion medium and carry out the immersion exposure at higher speed with a high-speed scan, while maintaining the lithography properties of the radiation-sensitive resin composition. As a result, the hydrophobicity of the surface of the resist film can be improved, resulting in the prevention of the defect due to the immersion, for example, the watermark defect. Example of the compound which may be used as the localization enhancing agent includes a low molecular weight compound having a specific dielectric constant of not less than 30 and not more than 200 and a boiling point of 100° C. or more at 1 atm. Specific examples of the compound include a lactone compound, a carbonate compound, a nitrile compound, and a polyhydric alcohol.

Examples of the lactone compound include γ-butyrolactone, valerolactone, mevaloniclactone, and norbornane lactone.

Examples of the carbonate compound include propylene carbonate, ethylene carbonate, butylene carbonate, and vinylene carbonate.

Example of the nitrile compound includes succinonitrile.

Example of the polyhydric alcohol includes glycerine.

The lower limit of the content of the localization enhancing agent is preferably 10 parts by mass, more preferably 15 parts by mass, further preferably 20 parts by mass, and more further preferably 25 parts by mass based on 100 parts by mass of total resins in the radiation-sensitive resin composition. The upper limit of the content is preferably 300 parts by mass, more preferably 200 parts by mass, further preferably 100 parts by mass, and more further preferably 80 parts by mass. The radiation-sensitive resin composition may include one type of the localization enhancing agent, or two or more types of localization enhancing agents in combination.

### (Surfactant)

The surfactant has an effect of improving the coating properties, the striation, and the developability of the composition. Examples of the surfactant include a nonionic surfactant, including polyoxyethylene lauryl ether, polyoxyethylene stearyl ether, polyoxyethylene oleyl ether, polyoxyethylene n-octylphenyl ether, polyoxyethylene n-nonylphenyl ether, polyethylene glycol dilaurate, and polyethylene glycol distearate. Examples of the surfactant which is commercially available include KP341 (manufactured by Shin-Etsu Chemical Co., Ltd.), POLYFLOW No. 75, POLYFLOW No. 95 (all manufactured by Kyoeisha Chemical Co., Ltd.), EFTOP EF301, EFTOP EF303, EFTOP EF352 (all manufactured by Tokem Products), Megafac F171, Megafac F173 (all manufactured by DIC), Fluorad FC430, Fluorad FC431 (all manufactured by Sumitomo 3M Limited), AsahiGuard AG710, Surflon S-382, Surflon SC-101, Surflon SC-102, Surflon SC-103, Surflon SC-104, Surflon SC-105, Surflon SC-106 (all manufactured by Asahi Glass Co., Ltd.). The content of the surfactant in the radiation-sensitive resin composition is typically not more than 2 parts by mass based on 100 parts by mass of total resins.

### (Alicyclic Backbone-Containing Compound)

The alicyclic backbone-containing compound has an effect of improving the dry etching resistance, the shape of the pattern, the adhesiveness between the substrate, and the like.

Examples of the alicyclic backbone-containing compound include:

adamantane derivatives, including 1-adamantane carboxylic acid, 2-adamantanone, and t-butyl 1-adamantane carboxylate;

deoxycholic acid esters, including t-butyl deoxycholate, t-butoxycarbonylmethyl deoxycholate, and 2-ethoxyethyl deoxycholate;

lithocholic acid esters, including t-butyl lithocholate, t-butoxycarbonylmethyl lithocholate, and 2-ethoxyethyl lithocholate; and

3-[2-hydroxy-2,2-bis(trifluoromethyl)ethyl]tetracyclo[4.4.0.1 (2,5) 0.1 (7,10)]dode cane, and 2-hydroxy-9-methoxycarbonyl-5-oxo-4-oxa-tricyclo[4.2.1.0 (3,7)]nonane. The content of the alicyclic backbone-containing compound in the radiation-sensitive resin composition is typically not more than 5 parts by mass based on 100 parts by mass of total resins.

### (Sensitizer)

The sensitizer shows an action of increasing the production of the acid, for example, from the radiation-sensitive acid generator, and has an effect of improving the “apparent sensitivity” of the radiation-sensitive resin composition.

Examples of the sensitizer include carbazoles, acetophenones, benzophenones, naphthalenes, phenols, biacetyl, eosin, rose bengal, pyrenes, anthracenes, and phenothiazines. The sensitizer may be used alone, or two or more sensitizers may be used in combination. The content of the sensitizer in the radiation-sensitive resin composition is typically not more than 2 parts by mass based on 100 parts by mass of total resins.

<Method for Preparing Radiation-Sensitive Resin Composition>

The radiation-sensitive resin composition can be prepared by, for example, mixing the compound (1), the resin, the radiation-sensitive acid generator, and optionally the high fluorine-content resin, as well as the solvent added in a predetermined ratio. The radiation-sensitive resin composition is preferably filtered through, for example, a filter having a pore diameter of about 0.05 μm to 0.20 μm after mixing. The solid matter concentration of the radiation-sensitive resin composition is usually 0.1 mass % to 50 mass %, preferably 0.5 mass % to 30 mass %, more preferably 1 mass % to 20 mass %.

<Method for Forming Resist Pattern>

A method for forming a resist pattern according to one embodiment of the present embodiment includes:

(1) applying the radiation-sensitive resin composition directly or indirectly on a substrate to form a resist film from (hereinafter, also referred to as a “resist film forming step”);

(2) exposing the resist film (hereinafter, also referred to as an “exposure step”); and

(3) developing the exposed resist film (hereinafter, also referred to as a “development step”).

The method for forming a resist pattern uses the above-described radiation-sensitive resin composition excellent in sensitivity in the exposure step, CDU performance, and LWR performance, and therefore a high-quality resist pattern can be formed. Hereinbelow, each of the steps will be described.

[Resist Film Forming Step]

In this step (the above mentioned step (1)), a resist film is formed with the radiation-sensitive resin composition. Examples of the substrate on which the resist film is formed include one traditionally known in the art, including a silicon wafer, silicon dioxide, and a wafer coated with aluminum. An organic or inorganic antireflection film may be formed on the substrate, as disclosed in JP-B-06-12452 and JP-A-59-93448. Examples of the applicating method include a rotary coating (spin coating), flow casting, and roll coating. After applicating, a prebake (PB) may be carried out in order to evaporate the solvent in the film, if needed. The temperature of PB is typically from 60° C. to 140° C., and preferably from 80° C. to 120° C. The duration of PB is typically from 5 seconds to 600 seconds, and preferably from 10 seconds to 300 seconds. The thickness of the resist film formed is preferably from 10 nm to 1,000 nm, and more preferably from 10 nm to 500 nm.

When the immersion exposure is carried out, irrespective of presence of a water repellent polymer additive such as the high fluorine-content resin in the radiation-sensitive resin composition, the formed resist film may have a protective film for the immersion which is not soluble into the immersion liquid on the film in order to prevent a direct contact between the immersion liquid and the resist film. As the protective film for the immersion, a solvent-removable protective film that is removed with a solvent before the developing step (for example, see JP-A-2006-227632); or a developer-removable protective film that is removed during the development of the developing step (for example, see WO2005-069076 and WO2006-035790) may be used. In terms of the throughput, the developer-removable protective film is preferably used.

When the next step, the exposure step, is performed with radiation having a wavelength of 50 nm or less, it is preferable to use a resin having the structural unit (I) and the structural unit (III) as the base resin in the composition.

[Exposing Step]

In this step (the above mentioned step (2)), the resist film formed in the resist film forming step as the step (1) is exposed by irradiating with a radioactive ray through a photomask (optionally through an immersion medium such as water). Examples of the radioactive ray used for the exposure include visible ray, ultraviolet ray, far ultraviolet ray, extreme ultraviolet ray (EUV); an electromagnetic wave including X ray and γ ray; an electron beam; and a charged particle radiation such as α ray. Among them, far ultraviolet ray, an electron beam, or EUV is preferred. ArF excimer laser light (wavelength is 193 nm), KrF excimer laser light (wavelength is 248 nm), an electron beam, or EUV is more preferred. An electron beam or EUV having a wavelength of 50 nm or less which is identified as the next generation exposing technology is further preferred.

When the exposure is carried out by immersion exposure, examples of the immersion liquid include water and fluorine-based inert liquid. The immersion liquid is preferably a liquid which is transparent with respect to the exposing wavelength, and has a minimum temperature factor of the refractive index so that the distortion of the light image reflected on the film becomes minimum. However, when the exposing light source is ArF excimer laser light (wavelength is 193 nm), water is preferably used because of the ease of availability and ease of handling in addition to the above considerations. When water is used, a small proportion of an additive that decreases the surface tension of water and increases the surface activity may be added. Preferably, the additive cannot dissolve the resist film on the wafer and can neglect an influence on an optical coating at an under surface of a lens. The water used is preferably distilled water.

After the exposure, post exposure bake (PEB) is preferably carried out to promote the dissociation of the acid-dissociable group in the resin by the acid generated from the radiation-sensitive acid generator with the exposure in the exposed part of the resist film. The difference of solubility into the developer between the exposed part and the non-exposed part is generated by the PEB. The temperature of PEB is typically from 50° C. to 180° C., and preferably from 80° C. to 130° C. The duration of PEB is typically from 5 seconds to 600 seconds, and preferably from 10 seconds to 300 seconds.

[Developing Step]

In this step (the above mentioned step (3)), the resist film exposed in the exposing step as the step (2) is developed. By this step, the predetermined resist pattern can be formed. After the development, the resist pattern is washed with a rinse solution such as water or alcohol, and the dried, in general.

Examples of the developer used for the development include, in the alkaline development, an alkaline aqueous solution obtained by dissolving at least one alkaline compound such as sodium hydroxide, potassium hydroxide, sodium carbonate, sodium silicate, sodium metasilicate, ammonia water, ethylamine, n-propylamine, diethylamine, di-n-propylamine, triethylamine, methyldiethylamine, ethyldimethylamine, triethanolamine, tetramethyl ammonium hydroxide (TMAH), pyrrole, piperidine, choline, 1,8-diazabicyclo-[5.4.0]-7-undecene, 1,5-diazabicyclo-[4.3.0]-5-nonene. Among them, an aqueous TMAH solution is preferred, and 2.38% by mass of aqueous TMAH solution is more preferred.

In the case of the development with organic solvent, examples of the solvent include an organic solvent, including a hydrocarbon-based solvent, an ether-based solvent, an ester-based solvent, a ketone-based solvent, and an alcohol-based solvent; and a solvent containing an organic solvent. Examples of the organic solvent include one, two or more solvents listed as the solvent for the radiation-sensitive resin composition. Among them, an ester-based solvent or a ketone-based solvent is preferred. The ester-based solvent is preferably an acetate ester-based solvent, and more preferably n-butyl acetate or amyl acetate. The ketone-based solvent is preferably a chain ketone, and more preferably 2-heptanone. The content of the organic solvent in the developer is preferably not less than 80% by mass, more preferably not less than 90% by mass, further preferably not less than 95% by mass, and particularly preferably not less than 99% by mass. Examples of the ingredient other than the organic solvent in the developer include water and silicone oil.

Examples of the developing method include a method of dipping the substrate in a tank filled with the developer for a given time (dip method); a method of developing by putting and leaving the developer on the surface of the substrate with the surface tension for a given time (paddle method); a method of spraying the developer on the surface of the substrate (spray method); and a method of injecting the developer while scanning an injection nozzle for the developer at a constant rate on the substrate rolling at a constant rate (dynamic dispense method).

### EXAMPLES

Hereinafter, the present invention will be specifically described with reference to Examples, but the present invention is not limited to these Examples. Methods for measuring various physical property values are shown below.

### [Weight Average Molecular Weight (Mw) and Number Average Molecular Weight (Mn)]

The Mw and Mn of the polymer were measured under the conditions described above. The polydispersity (Mw/Mn) was calculated from the measurement results of Mw and Mn.

### [¹³C-NMR Analysis]

¹³C-NMR analysis of the polymer was performed using a nuclear magnetic resonance apparatus (“JNM-Delta 400” manufactured by JEOL Ltd.).

### <Synthesis of Resin and High Fluorine-Content Resin>

The monomers used in the synthesis of each resin and high fluorine-content resin in Examples and Comparative Examples are shown below. In the following synthesis examples, unless otherwise specified, parts by mass means a value when the total mass of monomers used is 100 parts by mass, and mol % means a value when the total number of moles of monomers used is 100 mol %.

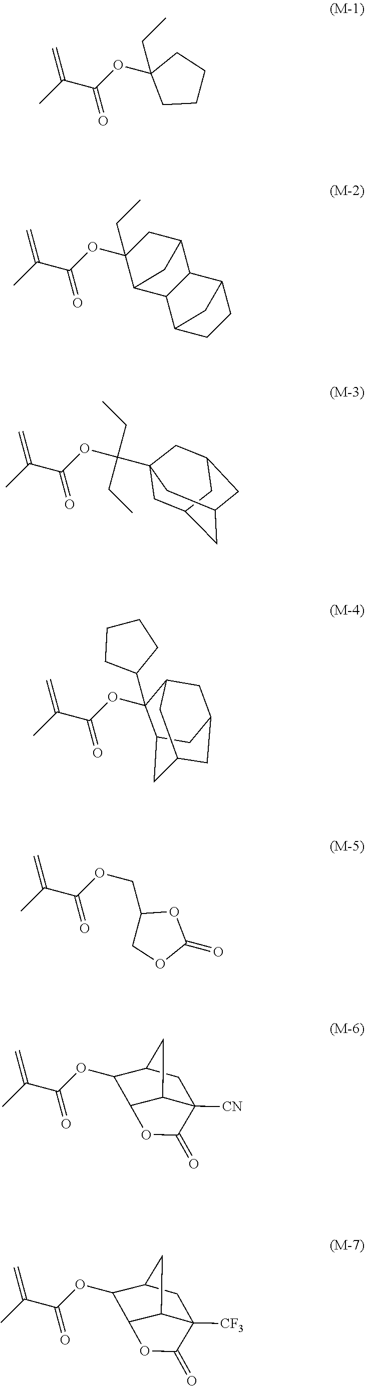

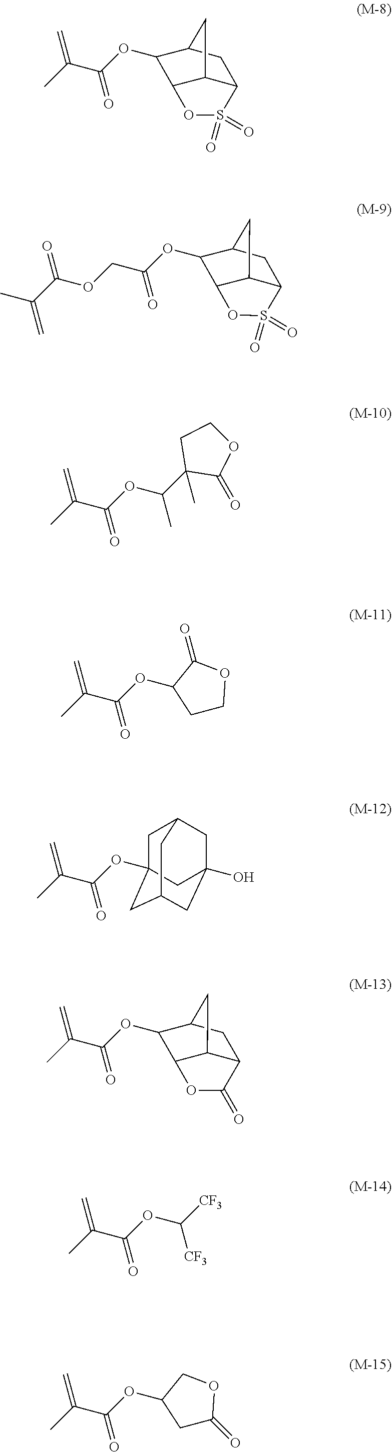

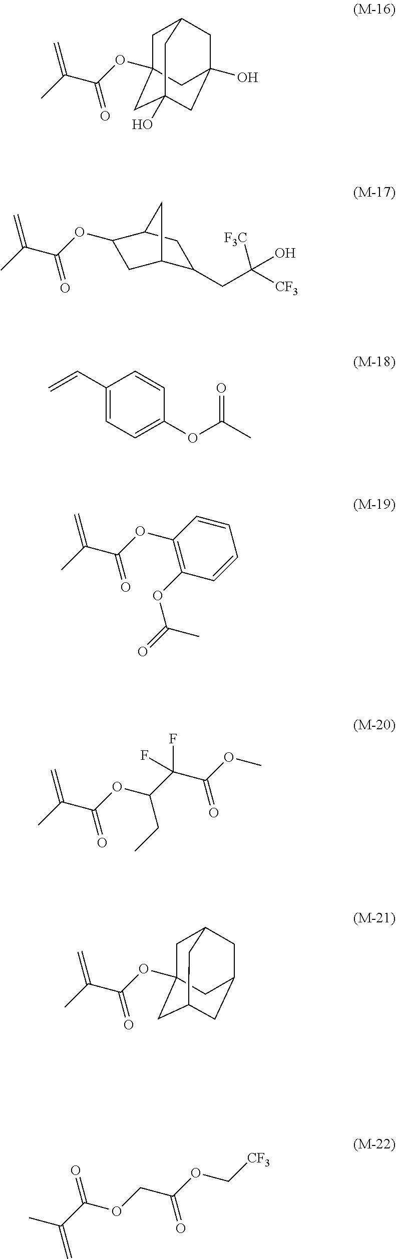

### Synthesis Example 1

### Synthesis of Resin (A-1)

The monomer (M−1), the monomer (M−2), and the monomer (M−10) were dissolved in 2-butanone (200 parts by mass) so as to have a molar ratio of 40/15/45 (mol %), and AIBN (azobisisobutyronitrile) (3 mol % with respect to 100 mol % in total of the used monomers) was added as an initiator to prepare a monomer solution. A reaction vessel was charged with 2-butanone (100 parts by mass) and purged with nitrogen for 30 minutes, and inside of the reaction vessel was adjusted to 80° C. Then, the monomer solution was added dropwise thereto over 3 hours with stirring. The polymerization reaction was performed for 6 hours with the start of dropwise addition as the initiation time of the polymerization reaction. After completion of the polymerization reaction, the polymerization solution was cooled to 30° C. or lower by water cooling. The cooled polymerization solution was added to methanol (2,000 parts by mass), and the precipitated white powder was separated by filtration. The separated white powder was washed with methanol twice, then separated by filtration, and dried at 50° C. for 24 hours to obtain a white powdery resin (A-1) (yield: 80%). The resin (A-1) had a Mw of 8,700 and a Mw/Mn of 1.49. As a result of ¹³C-NMR analysis, the contents of the structural units derived from (M−1), (M−2), and (M−10) were 39.9 mol %, 14.3 mol %, and 45.8 mol %, respectively.

### Synthesis Examples 2 to 11

### Synthesis of Resins (A-2) to (A-11)

Resins (A-2) to (A-11) were synthesized in the same manner as in Synthesis Example 1 except that monomers of types and blending ratios shown in the following Table 1 were used. The content (mol %), yield (%), and physical property values (Mw and Mw/Mn) of each structural unit of the obtained resins are shown together in the following Table 1. Incidentally, “−” in the following Table 1 indicates that the corresponding monomer was not used.

TABLE 1

| | | | | | | | | MONOMER PROVIDING | | | | |
|---|---|---|---|---|---|---|---|---|---|---|---|---|
| | | | | | | | | STRUCTURAL UNIT | | | | |
| | | | | | | | | OTHER THAN | | | | |
| | | MONOMER PROVIDING | | | MONOMER PROVIDING | | | STRUCTURAL UNITS (I) AND | | | | |
| | | STRUCTURAL UNIT (I) | | | STRUCTURAL UNIT (II) | | | STRUCTURAL UNIT (II) | | | | |
| | | | | STRUC- | | | STRUC- | | | STRUC- | | |
| | | | BLEND- | TURAL | | BLEND- | TURAL | | BLEND- | TURAL | | |
| | | | ING | UNIT | | ING | UNIT | | ING | UNIT | | |
| | | | RATIO | CONTENT | | RATIO | CONTENT | | RATIO | CONTENT | | Mw/ |
| | RESIN | TYPE | (mol %) | (mol %) | TYPE | (mol %) | (mol %) | TYPE | (mol %) | (mol %) | Mw | Mn |
| SYNTHESIS | A-1 | M-1 | 40 | 39.9 | M-10 | 45 | 45.8 | — | — | — | 8700 | 1.49 |
| EXAMPLE | | M-2 | 15 | 14.3 | | | | | | | | |
| SYNTHESIS | A-2 | M-1 | 30 | 31.4 | M-15 | 60 | 60.6 | — | — | — | 9000 | 1.44 |
| EXAMPLE | | M-2 | 10 | 8.0 | | | | | | | | |
| SYNTHESIS | A-3 | M-1 | 30 | 31.9 | M-11 | 60 | 61.7 | — | — | — | 8900 | 1.39 |
| EXAMPLE | | M-3 | 10 | 6.4 | | | | | | | | |
| SYNTHESIS | A-4 | M-1 | 35 | 32.3 | M-13 | 45 | 49.6 | — | — | — | 8500 | 1.59 |
| EXAMPLE | | M-3 | 20 | 18.1 | | | | | | | | |
| SYNTHESIS | A-5 | M-1 | 40 | 41.1 | M-9 | 45 | 45.7 | — | — | — | 8700 | 1.44 |
| EXAMPLE | | M-4 | 15 | 13.2 | | | | | | | | |
| SYNTHESIS | A-6 | M-1 | 40 | 41.6 | M-8 | 45 | 46.1 | — | — | — | 7700 | 1.51 |
| EXAMPLE | | M-4 | 15 | 12.3 | | | | | | | | |
| SYNTHESIS | A-7 | M-1 | 40 | 42.4 | M-7 | 45 | 39.5 | M-12 | 15 | 18.1 | 7800 | 1.59 |
| EXAMPLE | | | | | | | | | | | | |
| SYNTHESIS | A-8 | M-1 | 40 | 41.1 | M-6 | 40 | 35.7 | M-16 | 20 | 23.2 | 8100 | 1.56 |
| EXAMPLE 8 | | | | | | | | | | | | |
| SYNTHESIS | A-9 | M-1 | 50 | 51.0 | M-5 | 50 | 49.0 | — | — | — | 7800 | 1.55 |
| EXAMPLE 9 | | | | | | | | | | | | |
| SYNTHESIS | A-10 | M-1 | 40 | 44.4 | M-13 | 60 | 55.6 | — | — | — | 7900 | 1.59 |
| EXAMPLE 10 | | | | | | | | | | | | |
| SYNTHESIS | A-11 | M-1 | 40 | 42.8 | M-6 | 60 | 57.2 | — | — | — | 8000 | 1.43 |
| EXAMPLE 11 | | | | | | | | | | | | |

### Synthesis Example 12

### Synthesis of Resin (A-12)

The monomer (M−1) and the monomer (M−18) were dissolved in 1-methoxy-2-propanol (200 parts by mass) so as to have a molar ratio of 50/50 (mol %), and AIBN (5 mol %) was added as an initiator to prepare a monomer solution. A reaction vessel was charged with 1-methoxy-2-propanol (100 parts by mass) and purged with nitrogen for 30 minutes, and inside of the reaction vessel was adjusted to 80° C. Then, the monomer solution was added dropwise thereto over 3 hours with stirring. The polymerization reaction was performed for 6 hours with the start of dropwise addition as the initiation time of the polymerization reaction. After completion of the polymerization reaction, the polymerization solution was cooled to 30° C. or lower by water cooling. The cooled polymerization solution was added to hexane (2,000 parts by mass), and the precipitated white powder was separated by filtration. The separated white powder was washed twice with hexane, then separated by filtration, and dissolved in 1-methoxy-2-propanol (300 parts by mass). Subsequently, methanol (500 parts by mass), triethylamine (50 parts by mass), and ultrapure water (10 parts by mass) were added, and a hydrolysis reaction was performed at 70° C. for 6 hours with stirring. After completion of the reaction, the remaining solvent was distilled off, and the obtained solid was dissolved in acetone (100 parts by mass). The solution was added dropwise to water (500 parts by mass) to solidify the resin. The resulting solid was separated by filtration and dried at 50° C. for 13 hours to obtain a white powdery resin (A-12) (yield: 79%). The resin (A-12) had a Mw of 5,200 and a Mw/Mn of 1.60. As a result of ¹³C-NMR analysis, the contents of the structural units derived from (M−1) and (M−18) were 51.3 mol % and 48.7 mol %, respectively.

### Synthesis Examples 13 to 15

### Synthesis of Resins (A-13) to (A-15)

Resins (A-13) to (A-15) were synthesized in the same manner as in Synthesis Example 12 except that monomers of types and blending ratios shown in the following Table 2 were used. The content (mol %), yield (%), and physical property values (Mw and Mw/Mn) of each structural unit of the obtained resins are also shown in the following Table 2.

TABLE 2

| | | | | | | | | MONOMER PROVIDING | | | | |
|---|---|---|---|---|---|---|---|---|---|---|---|---|
| | | | | | | | | STRUCTURAL UNIT | | | | |
| | | | | | | | | OTHER THAN | | | | |
| | | MONOMER PROVIDING | | | MONOMER PROVIDING | | | STRUCTURAL UNITS (I) AND | | | | |
| | | STRUCTURAL UNIT (I) | | | STRUCTURAL UNIT (II) | | | STRUCTURAL UNIT (II) | | | | |
| | | | | STRUC- | | | STRUC- | | | STRUC- | | |
| | | | BLEND- | TURAL | | BLEND- | TURAL | | BLEND- | TURAL | | |
| | | | ING | UNIT | | ING | UNIT | | ING | UNIT | | |
| | | | RATIO | CONTENT | | RATIO | CONTENT | | RATIO | CONTENT | | Mw/ |
| | RESIN | TYPE | (mol %) | (mol %) | TYPE | (mol %) | (mol %) | TYPE | (mol %) | (mol %) | Mw | Mn |
| SYNTHESIS | A-12 | M-1 | 50 | 51.3 | — | — | — | M-18 | 50 | 48.7 | 5200 | 1.60 |
| EXAMPLE 12 | | | | | | | | | | | | |
| SYNTHESIS | A-13 | M-3 | 50 | 47.7 | M-16 | 20 | 20.1 | M-19 | 30 | 32.2 | 5800 | 1.51 |
| EXAMPLE 13 | | | | | | | | | | | | |
| SYNTHESIS | A-14 | M-2 | 50 | 48.1 | M-17 | 20 | 21.3 | M-18 | 30 | 30.6 | 5100 | 1.59 |
| EXAMPLE 14 | | | | | | | | | | | | |
| SYNTHESIS | A-15 | M-1 | 55 | 54.3 | M-17 | 15 | 15.6 | M-19 | 30 | 30.1 | 6200 | 1.53 |
| EXAMPLE 15 | | | | | | | | | | | | |

### Synthesis Example 16

### Synthesis of High Fluorine-Content Resin (E-1)

The monomer (M−1) and the monomer (M−20) were dissolved in 2-butanone (200 parts by mass) so as to have a molar ratio of 20/80 (mol %), and AIBN (4 mol %) was added as an initiator to prepare a monomer solution. A reaction vessel was charged with 2-butanone (100 parts by mass) and purged with nitrogen for 30 minutes, and inside of the reaction vessel was adjusted to 80° C. Then, the monomer solution was added dropwise thereto over 3 hours with stirring. The polymerization reaction was performed for 6 hours with the start of dropwise addition as the initiation time of the polymerization reaction. After completion of the polymerization reaction, the polymerization solution was cooled to 30° C. or lower by water cooling. The operation of replacing the solvent with acetonitrile (400 parts by mass), then adding hexane (100 parts by mass), stirring the mixture, and recovering the acetonitrile layer was repeated three times. The solvent was replaced with propylene glycol monomethyl ether acetate to obtain a solution of a high fluorine-content resin (E-1) (yield: 69%). The high fluorine-content resin (E-1) had a Mw of 6,000 and a Mw/Mn of 1.62. As a result of ¹³C-NMR analysis, the contents of the structural units derived from (M−1) and (M−20) were 19.9 mol % and 80.1 mol %, respectively.

### Synthesis Examples 17 to 20

### Synthesis of High Fluorine-Content Resins (E-2) to (E-5)

High fluorine-content resins (E-2) to (E-5) were synthesized in the same manner as in Synthesis Example 16 except that monomers of the types and blending ratios shown in the following Table 3 were used. The content (mol %), yield (%), and physical property values (Mw and Mw/Mn) of each structural unit of the obtained high fluorine-content resins are shown in the following Table 3.

TABLE 3

| | | MONOMER | | | MONOMER | | | |
|---|---|---|---|---|---|---|---|---|
| | | PROVIDING | | | PROVIDING | | | |
| | | STRUCTURAL UNIT | | | STRUCTURAL | | | |
| | | (VI) or (V) | | | UNIT (I) | | | |
| | HIGH | | | STRUC- | | | STRUC- | |
| | FLUOR- | | | TURAL | | | TURAL | |
| | INE- | | BLEND- | UNIT | | BLEND- | UNIT | |
| | CON- | | ING | CON- | | ING | CON- | |
| | TENT | | RATIO | TENT | | RATIO | TENT | |
| | RESIN | TYPE | (mol %) | (mol %) | TYPE | (mol %) | (mol %) | |
| SYNTHESIS | E-1 | M-20 | 80 | 80.1 | M-1 | 20 | 19.9 | |
| EXAMPLE 16 | | | | | | | | |
| SYNTHESIS | E-2 | M-22 | 80 | 81.9 | M-1 | 20 | 18.1 | |
| EXAMPLE 17 | | | | | | | | |
| SYNTHESIS | E-3 | M-14 | 60 | 62.3 | — | — | — | |
| EXAMPLE 18 | | | | | | | | |
| SYNTHESIS | E-4 | M-14 | 70 | 68.7 | — | — | — | |
| EXAMPLE 19 | | | | | | | | |
| SYNTHESIS | E-5 | M-14 | 70 | 12.3 | — | — | — | |
| EXAMPLE 20 | | | | | | | | |
| | MONOMER PROVIDING | | | | | | | |
| | STRUCTURAL UNIT | | | | | | | |
| | OTHER THAN | | | | | | | |
| | STRUCTURAL UNITS (I) AND | | | | | | | |
| | STRUCTURAL UNIT (II) | | | | | | | |
| | | | STRUC- | | | STRUC- | | |
| | | | TURAL | | | TURAL | | |
| | | BLEND- | UNIT | | BLEND- | UNIT | | |
| | | ING | CON- | | ING | CON- | | |
| | | RATIO | TENT | | RATIO | TENT | | Mw/ |
| | TYPE | (mol %) | (mol %) | TYPE | (mol %) | (mol %) | Mw | Mn |
| SYNTHESIS | — | — | — | — | — | — | 6000 | 1.62 |
| EXAMPLE 16 | | | | | | | | |
| SYNTHESIS | — | — | — | — | — | — | 7200 | 1.77 |
| EXAMPLE 17 | | | | | | | | |
| SYNTHESIS | — | — | — | M-21 | 40 | 38.7 | 6300 | 1.82 |
| EXAMPLE 18 | | | | | | | | |
| SYNTHESIS | M-12 | 30 | 31.3 | — | — | — | 6500 | 1.81 |
| EXAMPLE 19 | | | | | | | | |
| SYNTHESIS | M-17 | 30 | 27.7 | — | — | — | 6200 | 1.78 |
| EXAMPLE 20 | | | | | | | | |

### Synthesis of Compound (1)

### Synthesis Example 21

### Synthesis of Compound (C-1)

A reaction vessel was charged with 20.0 mmol of phenol, 30.0 mmol of ethyl bromodifluoroacetate, 30.0 mmol of 1,8-diazabicyclo[5,4,0]-7-undecene, and 50 g of dimethylformamide, and the mixture was stirred at 50° C. for 4 hours. Thereafter, the reaction solution was cooled to 30° C. or lower, diluted by adding water, and then extracted by adding ethyl acetate to separate the organic layer. The resulting organic layer was washed with a saturated aqueous solution of sodium chloride and then with water. After drying over sodium sulfate, the solvent was distilled off and purified by column chromatography to obtain a phenol derivative in good yield.

A mixed solution of methanol and water (1:1 (mass ratio)) was added to the phenol derivative to form a 1 M solution, then 20.0 mmol of sodium hydroxide was added thereto, and the mixture was reacted at 50° C. for 2 hours. Extraction was performed with acetonitrile, and the solvent was distilled off to obtain a sodium salt derivative. To the sodium salt derivative, 20.0 mmol of triphenylsulfonium chloride was added, and a mixed solution of water and dichloromethane (1:3 (mass ratio)) was added thereto. The mixture was vigorously stirred at room temperature for 3 hours, extraction was then performed by adding dichloromethane, and the organic layer was separated. The obtained organic layer was dried over sodium sulfate and the solvent was distilled off to obtain a compound (C-1) represented by the following formula (C-1) in good yield. The synthesis scheme of the compound (C-1) is shown below.

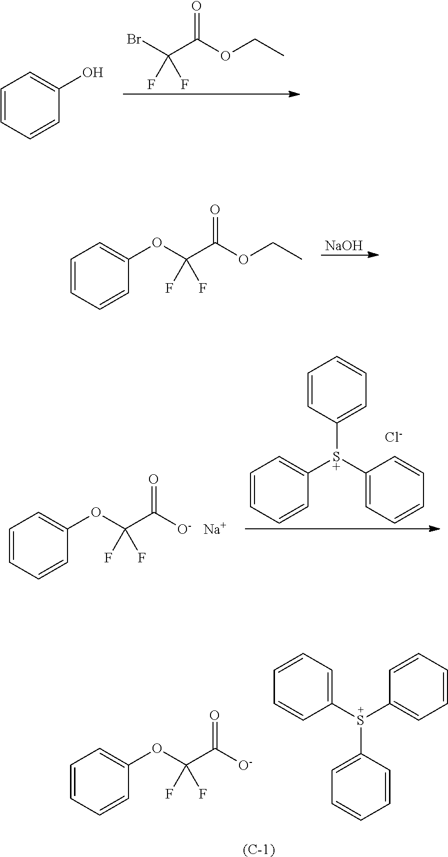

### Synthesis Examples 22 to 59

### Synthesis of Compounds (C-2) to (C-39)

Compounds (1) represented by the following formulas (C-2) to (C-39) were synthesized in the same manner as in Synthesis Example 21 except that the raw materials and the precursor were appropriately changed.

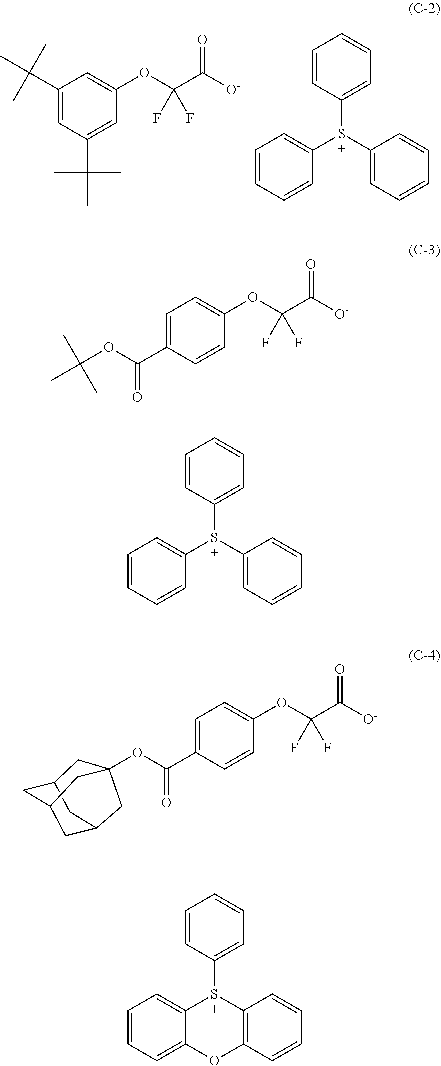

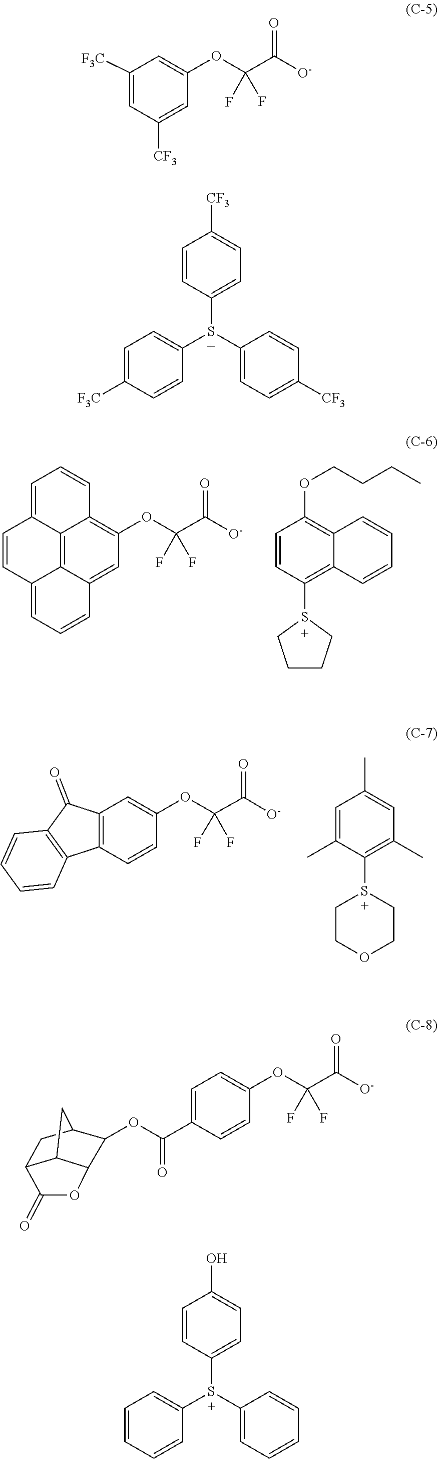

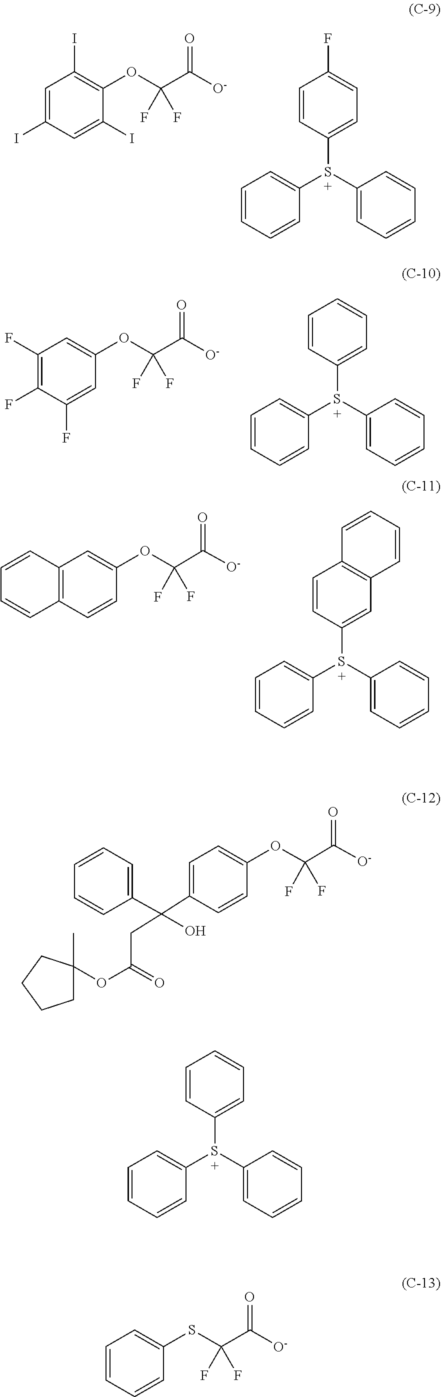

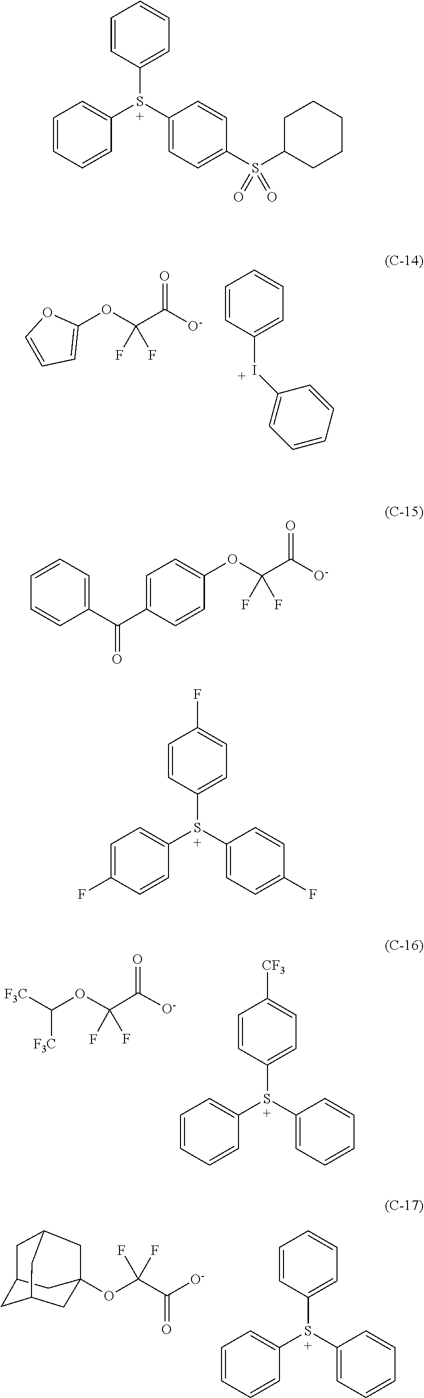

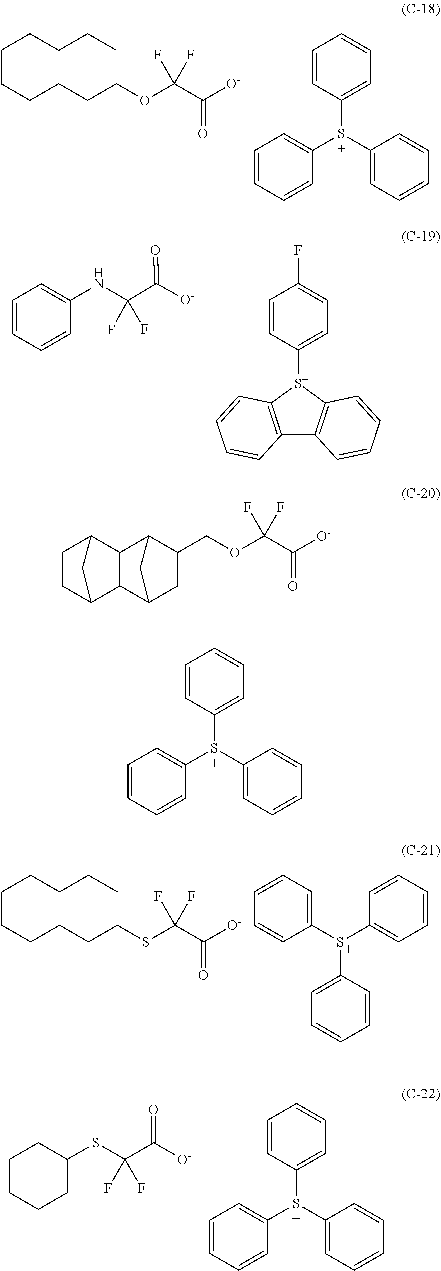

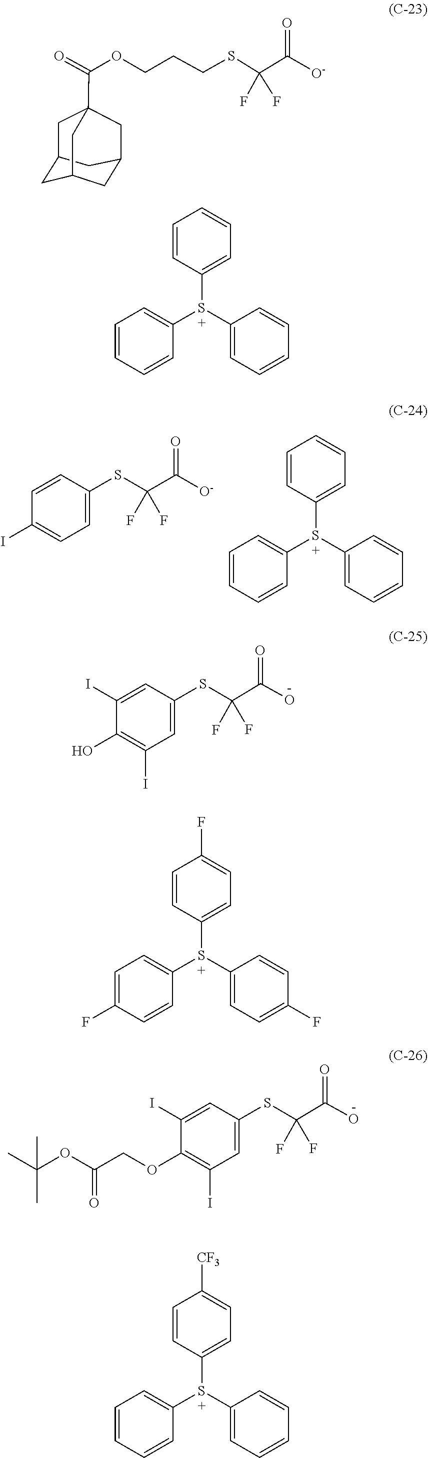

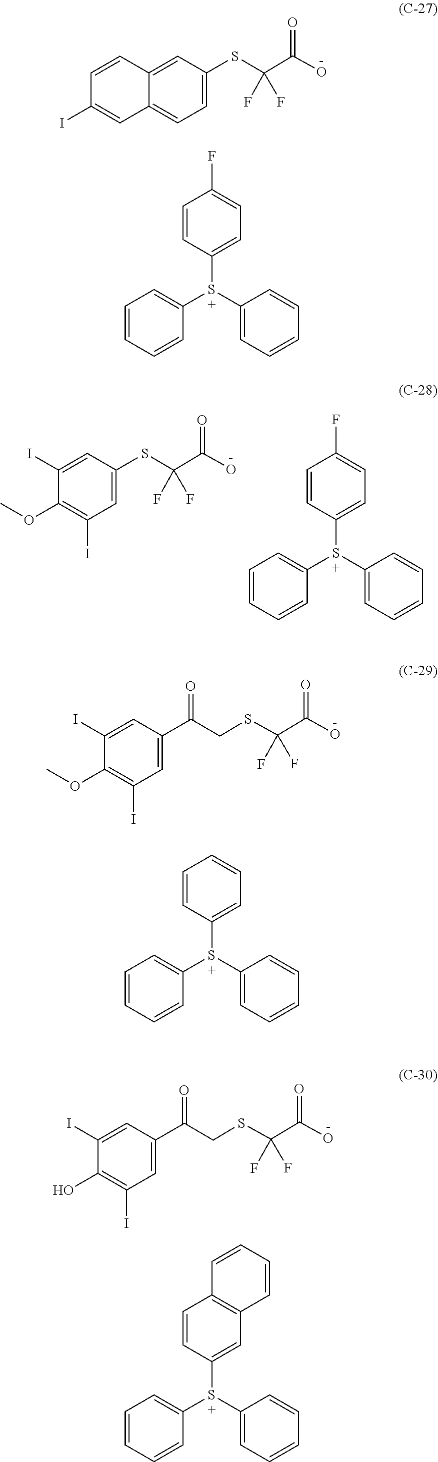

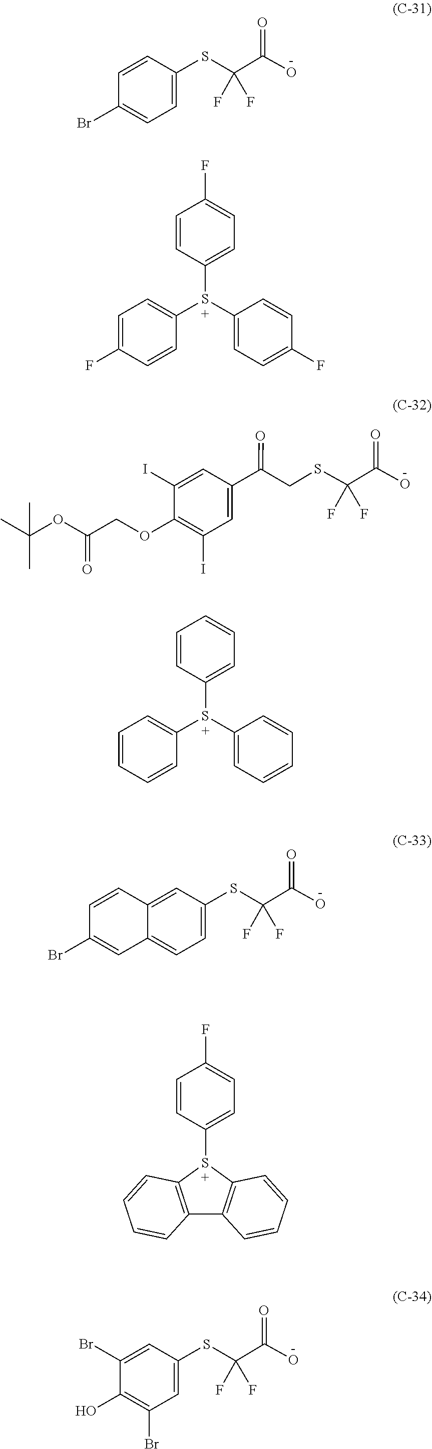

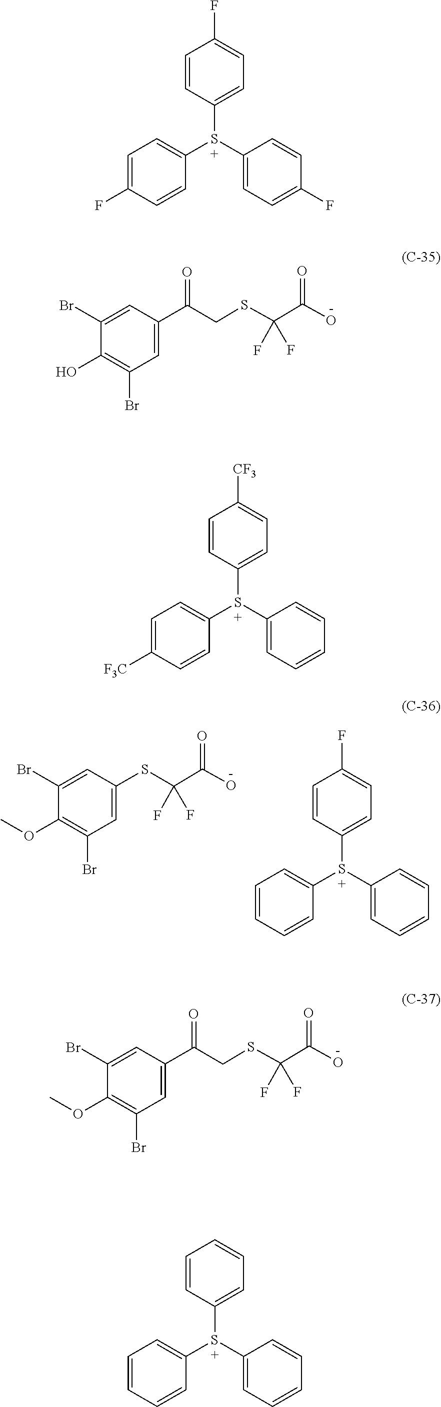

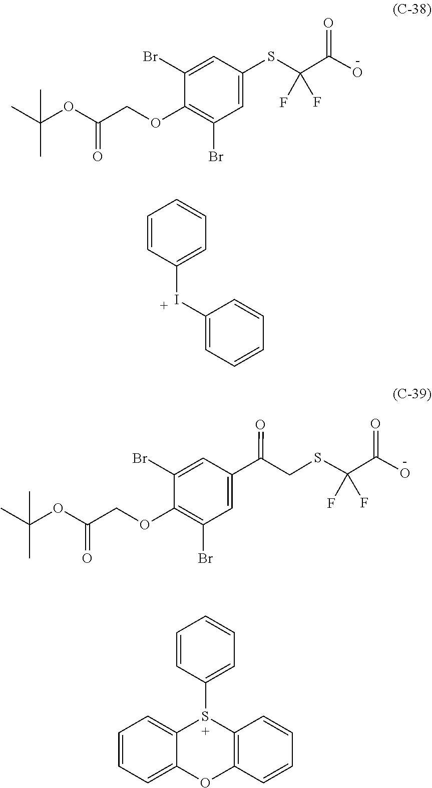

### <Preparation of Radiation-Sensitive Resin Composition>

Components other than the resin, the high fluorine-content resin, and the compound (1) used for preparing each radiation-sensitive resin composition are shown below.

### [Radiation-Sensitive Acid Generator]

B-1 to B-8: Compounds represented by the following formulas (B-1) to (B-8) (hereinafter, the compounds represented by the formulas (B-1) to (B-8) may be described as “radiation-sensitive acid generators (B-1) to (B-8)”, respectively.)

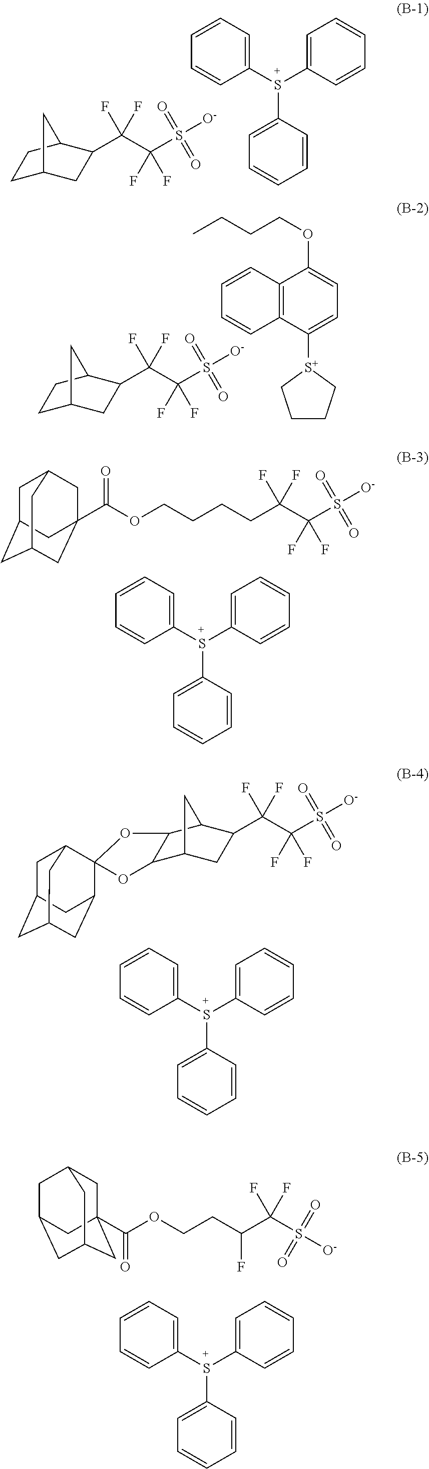

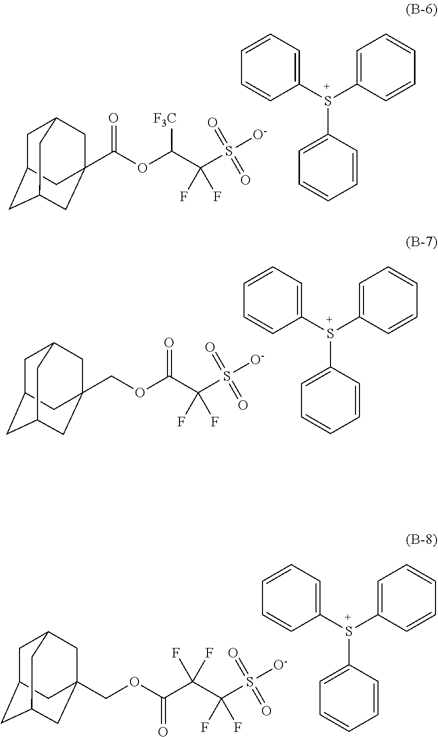

[Acid Diffusion Controlling Agent Other than Compounds (C-1) to (C-39)]

cc-1 to cc-10: Compounds represented by the following formulas (cc-1) to (cc-10) (hereinafter, the compounds represented by the formulas (cc-1) to (cc-10) may be described as “compounds (cc-1) to (cc-10)”, respectively.)

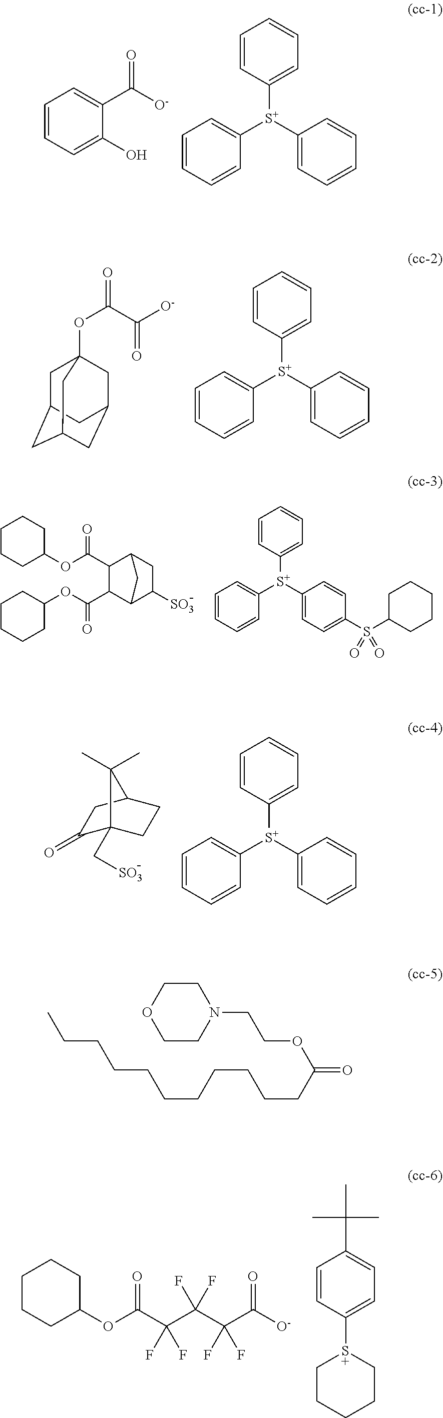

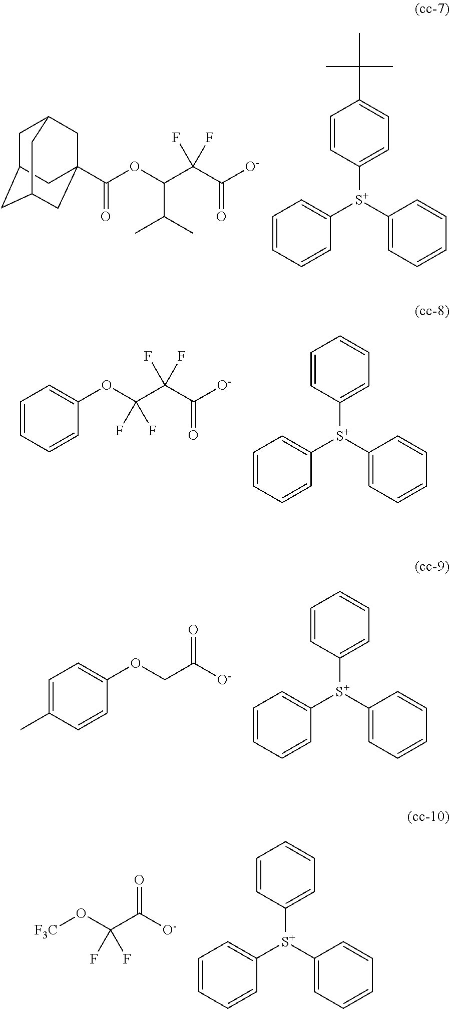

### [[D] Solvent]

D-1: Propylene glycol monomethyl ether acetate

D-2: Cyclohexanone

D-3: γ-Butyrolactone

D-4: Ethyl lactate

### [Preparation of Positive Radiation-Sensitive Resin Composition for ArF Exposure]

### Example 1

First, 100 parts by mass of (A-1) as a resin, 14.0 parts by mass of (B-4) as a radiation-sensitive acid generator, 2.3 parts by mass of (C-1) as the compound (1) (acid diffusion controlling agent), 5.0 parts by mass (solid content) of (E-1) as a high fluorine-content resin, and 3,230 parts by mass of a mixed solvent of (D-1)/(D-2)/(D-3)=70/29/1 (mass ratio) as a solvent were mixed, and the mixture was filtered through a membrane filter having a pore diameter of 0.2 μm to prepare a radiation-sensitive resin composition (J-1).

### Examples 2 to 48 and Comparative Examples 1 to 10

Radiation-sensitive resin compositions (J-2) to (J-48) and (CJ-1) to (CJ-10) were prepared in the same manner as in Example 1 except that the components of the types and contents shown in the following Table 4 were used.

TABLE 4

| | | | | | | | | HIGH | | | |
|---|---|---|---|---|---|---|---|---|---|---|---|
| | | | | | | | | FLUORINE- | | | |
| | | | | | | COMPOUND | | CONTENT | | | |
| | | | | RADIATION- | | (1) (ACID | | RESIN | | | |
| | | | | SENSITIVE | | DIFFUSION | | CONTROLLING | | | |
| | RADI- | RESIN | | ACID | | GENERATOR | | AGENT) | | SOLVENT | |
| | ATION- | | CON- | | CON- | | CON- | | CON- | | CON- |
| | SENSITIVE | | TENT | | TENT | | TENT | | TENT | | TENT |
| | RESIN | | (PARTS | | (PARTS | | (PARTS | | (PARTS | | (PARTS |
| | COMPO- | | BY | | BY | | BY | | BY | | BY |
| | SITION | TYPE | MASS) | TYPE | MASS) | TYPE | MASS) | TYPE | MASS) | TYPE | MASS) |
| EXAMPLE 1 | J-1 | A-1 | 100 | B-4 | 14.0 | C-1 | 2.3 | E-1 | 5.0 | D-1/D-2/D-3 | 2240/960/30 |
| EXAMPLE 2 | J-2 | A-1 | 100 | B-4 | 14.0 | C-2 | 2.3 | E-1 | 5.0 | D-1/D-2/D-3 | 2240/960/30 |
| EXAMPLE 3 | J-3 | A-1 | 100 | B-4 | 14.0 | C-3 | 2.3 | E-1 | 5.0 | D-1/D-2/D-3 | 2240/960/30 |
| EXAMPLE 4 | J-4 | A-1 | 100 | B-4 | 14.0 | C-4 | 2.3 | E-1 | 5.0 | D-1/D-2/D-3 | 2240/960/30 |
| EXAMPLE 5 | J-5 | A-1 | 100 | B-4 | 14.0 | C-5 | 2.3 | E-1 | 5.0 | D-1/D-2/D-3 | 2240/960/30 |
| EXAMPLE 6 | J-6 | A-1 | 100 | B-4 | 14.0 | C-6 | 2.3 | E-1 | 5.0 | D-1/D-2/D-3 | 2240/960/30 |
| EXAMPLE 7 | J-7 | A-1 | 100 | B-4 | 14.0 | C-7 | 2.3 | E-1 | 5.0 | D-1/D-2/D-3 | 2240/960/30 |
| EXAMPLE 8 | J-8 | A-1 | 100 | B-4 | 14.0 | C-8 | 2.3 | E-1 | 5.0 | D-1/D-2/D-3 | 2240/960/30 |
| EXAMPLE 9 | J-9 | A-1 | 100 | B-4 | 14.0 | C-9 | 2.3 | E-1 | 5.0 | D-1/D-2/D-3 | 2240/960/30 |
| EXAMPLE 10 | J-10 | A-1 | 100 | B-4 | 14.0 | C-10 | 2.3 | E-1 | 5.0 | D-1/D-2/D-3 | 2240/960/30 |
| EXAMPLE 11 | J-11 | A-1 | 100 | B-4 | 14.0 | C-11 | 2.3 | E-1 | 5.0 | D-1/D-2/D-3 | 2240/960/30 |
| EXAMPLE 12 | J-12 | A-1 | 100 | B-4 | 14.0 | C-12 | 2.3 | E-1 | 5.0 | D-1/D-2/D-3 | 2240/960/30 |
| EXAMPLE 13 | J-13 | A-1 | 100 | B-4 | 14.0 | C-13 | 2.3 | E-1 | 5.0 | D-1/D-2/D-3 | 2240/960/30 |
| EXAMPLE 14 | J-14 | A-1 | 100 | B-4 | 14.0 | C-14 | 2.3 | E-1 | 5.0 | D-1/D-2/D-3 | 2240/960/30 |
| EXAMPLE 15 | J-15 | A-1 | 100 | B-4 | 14.0 | C-15 | 2.3 | E-1 | 5.0 | D-1/D-2/D-3 | 2240/960/30 |
| EXAMPLE 16 | J-16 | A-1 | 100 | B-4 | 14.0 | C-16 | 2.3 | E-1 | 5.0 | D-1/D-2/D-3 | 2240/960/30 |
| EXAMPLE 17 | J-17 | A-1 | 100 | B-4 | 14.0 | C-17 | 2.3 | E-1 | 5.0 | D-1/D-2/D-3 | 2240/960/30 |
| EXAMPLE 18 | J-18 | A-1 | 100 | B-4 | 14.0 | C-18 | 2.3 | E-1 | 5.0 | D-1/D-2/D-3 | 2240/960/30 |
| EXAMPLE 19 | J-19 | A-1 | 100 | B-4 | 14.0 | C-19 | 2.3 | E-1 | 5.0 | D-1/D-2/D-3 | 2240/960/30 |
| EXAMPLE 20 | J-20 | A-1 | 100 | B-4 | 14.0 | C-20 | 2.3 | E-1 | 5.0 | D-1/D-2/D-3 | 2240/960/30 |
| EXAMPLE 21 | J-21 | A-1 | 100 | B-4 | 14.0 | C-21 | 2.3 | E-1 | 5.0 | D-1/D-2/D-3 | 2240/960/30 |
| EXAMPLE 22 | J-22 | A-1 | 100 | B-4 | 14.0 | C-22 | 2.3 | E-1 | 5.0 | D-1/D-2/D-3 | 2240/960/30 |
| EXAMPLE 23 | J-23 | A-1 | 100 | B-4 | 14.0 | C-23 | 2.3 | E-1 | 5.0 | D-1/D-2/D-3 | 2240/960/30 |
| EXAMPLE 24 | J-24 | A-1 | 100 | B-4 | 14.0 | C-24 | 2.3 | E-1 | 5.0 | D-1/D-2/D-3 | 2240/960/30 |
| EXAMPLE 25 | J-25 | A-2 | 100 | B-4 | 14.0 | C-1 | 2.3 | E-1 | 5.0 | D-1/D-2/D-3 | 2240/960/30 |
| EXAMPLE 26 | J-26 | A-3 | 100 | B-4 | 14.0 | C-1 | 2.3 | E-1 | 5.0 | D-1/D-2/D-3 | 2240/960/30 |
| EXAMPLE 27 | J-27 | A-4 | 100 | B-4 | 14.0 | C-1 | 2.3 | E-1 | 5.0 | D-1/D-2/D-3 | 2240/960/30 |
| EXAMPLE 28 | J-28 | A-5 | 100 | B-4 | 14.0 | C-1 | 2.3 | E-1 | 5.0 | D-1/D-2/D-3 | 2240/960/30 |
| EXAMPLE 29 | J-29 | A-6 | 100 | B-4 | 14.0 | C-1 | 2.3 | E-1 | 5.0 | D-1/D-2/D-3 | 2240/960/30 |
| EXAMPLE 30 | J-30 | A-7 | 100 | B-4 | 14.0 | C-1 | 2.3 | E-1 | 5.0 | D-1/D-2/D-3 | 2240/960/30 |
| EXAMPLE 31 | J-31 | A-8 | 100 | B-4 | 14.0 | C-1 | 2.3 | E-1 | 5.0 | D-1/D-2/D-3 | 2240/960/30 |
| EXAMPLE 32 | J-32 | A-9 | 100 | B-4 | 14.0 | C-1 | 2.3 | E-1 | 5.0 | D-1/D-2/D-3 | 2240/960/30 |
| EXAMPLE 33 | J-33 | A-10 | 100 | B-4 | 14.0 | C-1 | 2.3 | E-1 | 5.0 | D-1/D-2/D-3 | 2240/960/30 |
| EXAMPLE 34 | J-34 | A-11 | 100 | B-4 | 14.0 | C-1 | 2.3 | E-1 | 5.0 | D-1/D-2/D-3 | 2240/960/30 |
| EXAMPLE 35 | J-35 | A-1 | 100 | B-1 | 14.0 | C-1 | 2.3 | E-1 | 5.0 | D-1/D-2/D-3 | 2240/960/30 |
| EXAMPLE 36 | J-36 | A-1 | 100 | B-2 | 14.0 | C-1 | 2.3 | E-1 | 5.0 | D-1/D-2/D-3 | 2240/960/30 |
| EXAMPLE 37 | J-37 | A-1 | 100 | B-3 | 14.0 | C-1 | 2.3 | E-1 | 5.0 | D-1/D-2/D-3 | 2240/960/30 |
| EXAMPLE 38 | J-38 | A-1 | 100 | B-5 | 14.0 | C-1 | 2.3 | E-1 | 5.0 | D-1/D-2/D-3 | 2240/960/30 |
| EXAMPLE 39 | J-39 | A-1 | 100 | B-6 | 14.0 | C-1 | 2.3 | E-1 | 5.0 | D-1/D-2/D-3 | 2240/960/30 |
| EXAMPLE 40 | J-40 | A-1 | 100 | B-7 | 14.0 | C-1 | 2.3 | E-1 | 5.0 | D-1/D-2/D-3 | 2240/960/30 |
| EXAMPLE 41 | J-41 | A-1 | 100 | B-8 | 14.0 | C-1 | 2.3 | E-1 | 5.0 | D-1/D-2/D-3 | 2240/960/30 |
| EXAMPLE 42 | J-42 | A-1 | 100 | B-4 | 14.0 | C-1 | 2.3 | E-2 | 5.0 | D-1/D-2/D-3 | 2240/960/30 |
| EXAMPLE 43 | J-43 | A-1 | 100 | B-4 | 14.0 | C-1 | 2.3 | E-3 | 5.0 | D-1/D-2/D-3 | 2240/960/30 |
| EXAMPLE 44 | J-44 | A-1 | 100 | B-4 | 14.0 | C-1 | 2.3 | E-4 | 5.0 | D-1/D-2/D-3 | 2240/960/30 |
| EXAMPLE 45 | J-45 | A-1 | 100 | B-4 | 14.0 | C-1/cc-1 | 1.3/1.0 | E-1 | 5.0 | D-1/D-2/D-3 | 2240/960/30 |
| EXAMPLE 46 | J-46 | A-1 | 100 | B-4 | 14.0 | C-1/cc-4 | 1.3/1.0 | E-1 | 5.0 | D-1/D-2/D-3 | 2240/960/30 |
| EXAMPLE 47 | J-47 | A-1 | 100 | B-4/B-2 | 7.0/7.0 | C-1 | 2.3 | E-1 | 5.0 | D-1/D-2/D-3 | 2240/960/30 |
| EXAMPLE 48 | J-48 | A-1 | 100 | B-6/B-7 | 7.0/7.0 | C-1 | 2.3 | E-1 | 5.0 | D-1/D-2/D-3 | 2240/960/30 |
| COMPARATIVE | CJ-1 | A-1 | 100 | B-5 | 14.0 | cc-1 | 2.3 | E-1 | 5.0 | D-1/D-2/D-3 | 2240/960/30 |
| EXAMPLE 1 | | | | | | | | | | | |
| COMPARATIVE | CJ-2 | A-1 | 100 | B-5 | 14.0 | cc-2 | 2.3 | E-1 | 5.0 | D-1/D-2/D-3 | 2240/960/30 |
| EXAMPLE 2 | | | | | | | | | | | |
| COMPARATIVE | CJ-3 | A-1 | 100 | B-5 | 14.0 | cc-3 | 2.3 | E-1 | 5.0 | D-1/D-2/D-3 | 2240/960/30 |
| EXAMPLE 3 | | | | | | | | | | | |
| COMPARATIVE | CJ-4 | A-1 | 100 | B-5 | 14.0 | cc-4 | 2.3 | E-1 | 5.0 | D-1/D-2/D-3 | 2240/960/30 |
| EXAMPLE 4 | | | | | | | | | | | |
| COMPARATIVE | CJ-5 | A-1 | 100 | B-5 | 14.0 | cc-5 | 2.3 | E-1 | 5.0 | D-1/D-2/D-3 | 2240/960/30 |
| EXAMPLE 5 | | | | | | | | | | | |
| COMPARATIVE | CJ-6 | A-1 | 100 | B-5 | 14.0 | cc-6 | 2.3 | E-1 | 5.0 | D-1/D-2/D-3 | 2240/960/30 |
| EXAMPLE 6 | | | | | | | | | | | |
| COMPARATIVE | CJ-7 | A-1 | 100 | B-5 | 14.0 | cc-7 | 2.3 | E-1 | 5.0 | D-1/D-2/D-3 | 2240/960/30 |
| EXAMPLE 7 | | | | | | | | | | | |
| COMPARATIVE | CJ-8 | A-1 | 100 | B-5 | 14.0 | cc-8 | 2.3 | E-1 | 5.0 | D-1/D-2/D-3 | 2240/960/30 |
| EXAMPLE 8 | | | | | | | | | | | |
| COMPARATIVE | CJ-9 | A-1 | 100 | B-5 | 14.0 | cc-9 | 2.3 | E-1 | 5.0 | D-1/D-2/D-3 | 2240/960/30 |
| EXAMPLE 9 | | | | | | | | | | | |
| COMPARATIVE | CJ-10 | A-1 | 100 | B-5 | 14.0 | cc-10 | 2.3 | E-1 | 5.0 | D-1/D-2/D-3 | 2240/960/30 |
| EXAMPLE 10 | | | | | | | | | | | |

### <Formation of Resist Pattern Using Positive Radiation-Sensitive Resin Composition for ArF Exposure>

A composition for forming an underlayer antireflective film (“ARC66” manufactured by Brewer Science, Inc.) was applied onto a 12 inch silicon wafer using a spin coater (“CLEAN TRACK ACT12” manufactured by Tokyo Electron Limited), and then heated at 205° C. for 60 seconds to form an underlayer antireflective film having an average thickness of 105 nm. The positive radiation-sensitive resin composition for ArF exposure prepared above was applied onto the underlayer antireflective film using the spin coater, and subjected to PB (prebake) at 90° C. for 60 seconds. Thereafter, cooling was performed at 23° C. for 30 seconds to form a resist film having an average thickness of 90 nm. Subsequently, this resist film was exposed through a mask pattern of 40 nm space and 105 nm pitch using an ArF excimer laser immersion exposure apparatus (“TWINSCAN XT-1900i” manufactured by ASML Holding N.V.) under optical conditions of a numeral aperture (NA) of 1.35 and annular illumination (σ=0.8/0.6). After the exposure, PEB (post exposure bake) was performed at 90° C. for 60 seconds. Thereafter, the resist film was subjected to alkaline development using a 2.38 mass % aqueous TMAH solution as an alkaline developer. After the development, the resist film was washed with water and further dried to form a positive resist pattern (40 nm line-and-space pattern). In addition, a positive resist pattern (40 nm hole, 105 nm pitch) was formed in the same manner as in the above operation except that the mask pattern was changed.

### <Evaluation>

The sensitivity, LWR performance, and CDU performance of each of resist patterns formed using the positive radiation-sensitive resin compositions for ArF exposure were evaluated according to the following methods. The results are shown in the following Table 5. It is to be noted that a scanning electron microscope (“CG-5000” manufactured by Hitachi High-Tech Corporation) was used for measurement of the resist pattern.

### [Sensitivity]

An exposure dose at which a 40 nm line-and-space pattern was formed in formation of a resist pattern using the positive radiation-sensitive resin composition for ArF exposure was defined as an optimum exposure dose, and this optimum exposure dose was defined as sensitivity (mJ/cm²). A case where the sensitivity was 23 mJ/cm² or less was evaluated as “good”, and a case where the sensitivity exceeded 23 mJ/cm² was evaluated as “poor”.

### [CDU Performance]

A resist pattern with 40 nm holes and 105 nm pitches was measured using the scanning electron microscope, and measurement was performed at any 1,800 points in total from above the pattern. The dimensional variation (3σ) was determined and taken as the CDU performance (nm). A smaller value of CDU indicates smaller variation in the hole diameter in the long period and better performance. A case where the CDU performance was 3.3 nm or less was evaluated as “good”, and a case where the CDU performance exceeded 3.3 nm was evaluated as “poor”.

### [LWR Performance]

A resist pattern was formed with the mask size adjusted so as to form a 40 nm line-and-space pattern by irradiation with the optimum exposure dose obtained in the evaluation of the sensitivity. The formed resist pattern was observed from above the pattern using the scanning electron microscope. The variation in line width was measured at 500 points in total, the value of 3σ was obtained from the distribution of the measured values, and the value of 3σ was defined as LWR (nm). A smaller value of LWR indicates smaller roughness of the line and better performance. A case where the LWR performance was 3.6 nm or less was evaluated as “good”, and a case where the LWR performance exceeded 3.6 nm was evaluated as “poor”.

TABLE 5

| | RADIATION-SENSITIVE | SENSITIVITY | CDU | LWR |
|---|---|---|---|---|
| | RESIN COMPOSITION | (mJ/cm²) | (nm) | (nm) |
| EXAMPLE 1 | J-1 | 22.3 | 2.99 | 3.12 |
| EXAMPLE 2 | J-2 | 21.2 | 3.21 | 3.22 |
| EXAMPLE 3 | J-3 | 22.8 | 3.01 | 2.98 |
| EXAMPLE 4 | J-4 | 19.3 | 2.89 | 2.99 |
| EXAMPLE 5 | J-5 | 20.2 | 2.78 | 2.89 |
| EXAMPLE 6 | J-6 | 21.1 | 2.83 | 3.01 |
| EXAMPLE 7 | J-7 | 19.3 | 2.98 | 2.87 |
| EXAMPLE 8 | J-8 | 18.4 | 2.78 | 3.09 |
| EXAMPLE 9 | J-9 | 20.8 | 2.69 | 3.11 |
| EXAMPLE 10 | J-10 | 19.3 | 3.01 | 2.87 |
| EXAMPLE 11 | J-11 | 20.1 | 2.78 | 2.79 |
| EXAMPLE 12 | J-12 | 18.1 | 2.98 | 2.92 |
| EXAMPLE 13 | J-13 | 19.9 | 2.93 | 2.83 |
| EXAMPLE 14 | J-14 | 18.1 | 3.08 | 3.25 |
| EXAMPLE 15 | J-15 | 21.3 | 2.92 | 3.16 |
| EXAMPLE 16 | J-16 | 20.1 | 2.86 | 3.11 |
| EXAMPLE 17 | J-17 | 19.1 | 2.77 | 2.82 |
| EXAMPLE 18 | J-18 | 19.6 | 2.81 | 2.93 |
| EXAMPLE 19 | J-19 | 17.3 | 3.00 | 3.25 |
| EXAMPLE 20 | J-20 | 19.7 | 3.08 | 2.89 |
| EXAMPLE 21 | J-21 | 20.1 | 2.98 | 3.00 |
| EXAMPLE 22 | J-22 | 22.3 | 2.92 | 3.11 |
| EXAMPLE 23 | J-23 | 21.2 | 3.00 | 2.88 |
| EXAMPLE 24 | J-24 | 21.0 | 2.81 | 2.91 |
| EXAMPLE 25 | J-25 | 20.1 | 3.01 | 3.02 |
| EXAMPLE 26 | J-26 | 21.7 | 2.99 | 3.11 |
| EXAMPLE 27 | J-27 | 21.4 | 2.89 | 3.09 |
| EXAMPLE 28 | J-28 | 20.9 | 2.91 | 2.98 |
| EXAMPLE 29 | J-29 | 21.3 | 2.95 | 3.00 |
| EXAMPLE 30 | J-30 | 21.8 | 2.93 | 3.11 |
| EXAMPLE 31 | J-31 | 20.9 | 2.88 | 3.06 |
| EXAMPLE 32 | J-32 | 20.3 | 3.01 | 3.01 |
| EXAMPLE 33 | J-33 | 21.1 | 3.02 | 2.99 |
| EXAMPLE 34 | J-34 | 22.3 | 2.98 | 2.87 |
| EXAMPLE 35 | J-35 | 21.1 | 2.78 | 2.88 |
| EXAMPLE 36 | J-36 | 22.1 | 2.88 | 2.84 |
| EXAMPLE 37 | J-37 | 22.7 | 2.91 | 3.01 |
| EXAMPLE 38 | J-38 | 21.4 | 2.81 | 2.67 |
| EXAMPLE 39 | J-39 | 20.8 | 2.78 | 2.91 |
| EXAMPLE 40 | J-40 | 21.5 | 2.91 | 2.93 |
| EXAMPLE 41 | J-41 | 21.9 | 2.93 | 2.99 |
| EXAMPLE 42 | J-42 | 21.4 | 2.99 | 3.11 |
| EXAMPLE 43 | J-43 | 22.3 | 3.01 | 3.09 |
| EXAMPLE 44 | J-44 | 21.6 | 2.98 | 3.13 |
| EXAMPLE 45 | J-45 | 22.0 | 2.99 | 3.22 |
| EXAMPLE 46 | J-46 | 21.7 | 2.87 | 2.88 |
| EXAMPLE 47 | J-47 | 22.7 | 2.91 | 2.92 |
| EXAMPLE 48 | J-48 | 21.1 | 3.01 | 2.81 |
| COMPARATIVE EXAMPLE 1 | CJ-1 | 28.7 | 5.23 | 4.55 |
| COMPARATIVE EXAMPLE 2 | CJ-2 | 26.5 | 5.12 | 4.12 |
| COMPARATIVE EXAMPLE 3 | CJ-3 | 26.9 | 5.21 | 4.89 |
| COMPARATIVE EXAMPLE 4 | CJ-4 | 27.2 | 4.91 | 4.44 |
| COMPARATIVE EXAMPLE 5 | CJ-5 | 32.8 | 5.52 | 5.78 |
| COMPARATIVE EXAMPLE 6 | CJ-6 | 25.1 | 4.44 | 4.12 |
| COMPARATIVE EXAMPLE 7 | CJ-7 | 25.6 | 4.67 | 4.08 |
| COMPARATIVE EXAMPLE 8 | CJ-8 | 36.7 | 5.55 | 5.78 |
| COMPARATIVE EXAMPLE 9 | CJ-9 | 38.1 | 5.98 | 5.55 |
| COMPARATIVE EXAMPLE 10 | CJ-10 | 28.1 | 4.13 | 4.42 |

As is apparent from the results in Table 5, the radiation-sensitive resin compositions of Examples were superior in sensitivity, LWR performance, and CDU performance when used for ArF exposure, whereas the radiation-sensitive resin compositions of Comparative Examples were inferior in each characteristic to Examples. Therefore, when the radiation-sensitive resin compositions of Examples were used for ArF exposure, a resist pattern having high sensitivity and superior LWR performance and CDU performance can be formed.

### [Preparation of Positive Radiation-Sensitive Resin Composition for Extreme Ultraviolet (EUV) Exposure]

### Example 49

First, 100 parts by mass of (A-12) as a resin, 20.0 parts by mass of (B-4) as a radiation-sensitive acid generator, 3.2 parts by mass of (C-1) as the compound (1) (acid diffusion controlling agent), 3.0 parts by mass of (E-5) as a high fluorine-content resin, and 6,110 parts by mass of a mixed solvent of (D-1)/(D-4)=70/30 (mass ratio) as a solvent were mixed, and the mixture was filtered through a membrane filter having a pore diameter of 0.2 μm to prepare a radiation-sensitive resin composition (J-49).

### Examples 50 to 85 and Comparative Examples 11 to 14

Radiation-sensitive resin compositions (J-50) to (J-85) and (CJ-11) to (CJ-14) were prepared in the same manner as in Example 41 except that the components of the types and contents shown in the following Table 6 were used.

TABLE 6

| | | | | | | COMPOUND | | | | | |
|---|---|---|---|---|---|---|---|---|---|---|---|
| | | | | RADIATION- | | (1) (ACID | | HIGH | | | |
| | | | | SENSITIVE | | DIFFUSION | | FLUORINE- | | | |
| | | | | ACID | | CONTROLLING | | CONTENT | | | |
| | RADI- | RESIN | | GENERATOR | | AGENT) | | RESIN | | SOLVENT | |
| | ATION- | | CON- | | CON- | | CON- | | CON- | | CON- |
| | RESIN | | TENT | | TENT | | TENT | | TENT | | TENT |
| | SENSITIVE | | (PARTS | | (PARTS | | (PARTS | | (PARTS | | (PARTS |
| | COMPO- | | BY | | BY | | BY | | BY | | BY |
| | SITION | TYPE | MASS) | TYPE | MASS) | TYPE | MASS) | TYPE | MASS) | TYPE | MASS) |
| EXAMPLE 49 | J-49 | A-12 | 100 | B-4 | 20.0 | C-1 | 3.2 | E-5 | 3.0 | D-1/D-4 | 4280/1830 |
| EXAMPLE 50 | J-50 | A-12 | 100 | B-4 | 20.0 | C-5 | 3.2 | E-5 | 3.0 | D-1/D-4 | 4280/1830 |
| EXAMPLE 51 | J-51 | A-12 | 100 | B-4 | 20.0 | C-10 | 3.2 | E-5 | 3.0 | D-1/D-4 | 4280/1830 |
| EXAMPLE 52 | J-52 | A-12 | 100 | B-4 | 20.0 | C-14 | 3.2 | E-5 | 3.0 | D-1/D-4 | 4280/1830 |
| EXAMPLE 53 | J-53 | A-12 | 100 | B-4 | 20.0 | C-15 | 3.2 | E-5 | 3.0 | D-1/D-4 | 4280/1830 |
| EXAMPLE 54 | J-54 | A-12 | 100 | B-4 | 20.0 | C-22 | 3.2 | E-5 | 3.0 | D-1/D-4 | 4280/1830 |
| EXAMPLE 55 | J-55 | A-12 | 100 | B-4 | 20.0 | C-24 | 3.2 | E-5 | 3.0 | D-1/D-4 | 4280/1830 |
| EXAMPLE 56 | J-56 | A-12 | 100 | B-4 | 20.0 | C-25 | 3.2 | E-5 | 3.0 | D-1/D-4 | 4280/1830 |
| EXAMPLE 57 | J-57 | A-12 | 100 | B-4 | 20.0 | C-26 | 3.2 | E-5 | 3.0 | D-1/D-4 | 4280/1830 |
| EXAMPLE 58 | J-58 | A-12 | 100 | B-4 | 20.0 | C-27 | 3.2 | E-5 | 3.0 | D-1/D-4 | 4280/1830 |
| EXAMPLE 59 | J-59 | A-12 | 100 | B-4 | 20.0 | C-28 | 3.2 | E-5 | 3.0 | D-1/D-4 | 4280/1830 |
| EXAMPLE 60 | J-60 | A-12 | 100 | B-4 | 20.0 | C-29 | 3.2 | E-5 | 3.0 | D-1/D-4 | 4280/1830 |
| EXAMPLE 61 | J-61 | A-12 | 100 | B-4 | 20.0 | C-30 | 3.2 | E-5 | 3.0 | D-1/D-4 | 4280/1830 |
| EXAMPLE 62 | J-62 | A-12 | 100 | B-4 | 20.0 | C-31 | 3.2 | E-5 | 3.0 | D-1/D-4 | 4280/1830 |
| EXAMPLE 63 | J-63 | A-12 | 100 | B-4 | 20.0 | C-32 | 3.2 | E-5 | 3.0 | D-1/D-4 | 4280/1830 |
| EXAMPLE 64 | J-64 | A-12 | 100 | B-4 | 20.0 | C-33 | 3.2 | E-5 | 3.0 | D-1/D-4 | 4280/1830 |
| EXAMPLE 65 | J-65 | A-12 | 100 | B-4 | 20.0 | C-34 | 3.2 | E-5 | 3.0 | D-1/D-4 | 4280/1830 |
| EXAMPLE 66 | J-66 | A-12 | 100 | B-4 | 20.0 | C-35 | 3.2 | E-5 | 3.0 | D-1/D-4 | 4280/1830 |
| EXAMPLE 67 | J-67 | A-12 | 100 | B-4 | 20.0 | C-36 | 3.2 | E-5 | 3.0 | D-1/D-4 | 4280/1830 |
| EXAMPLE 68 | J-68 | A-12 | 100 | B-4 | 20.0 | C-37 | 3.2 | E-5 | 3.0 | D-1/D-4 | 4280/1830 |
| EXAMPLE 69 | J-69 | A-12 | 100 | B-4 | 20.0 | C-38 | 3.2 | E-5 | 3.0 | D-1/D-4 | 4280/1830 |
| EXAMPLE 70 | J-70 | A-12 | 100 | B-4 | 20.0 | C-39 | 3.2 | E-5 | 3.0 | D-1/D-4 | 4280/1830 |
| EXAMPLE 71 | J-71 | A-12 | 100 | B-4 | 20.0 | C-24 | 2.0 | E-5 | 3.0 | D-1/D-4 | 4280/1830 |
| EXAMPLE 72 | J-72 | A-12 | 100 | B-4 | 20.0 | C-24 | 6.0 | E-5 | 3.0 | D-1/D-4 | 4280/1830 |
| EXAMPLE 73 | J-73 | A-12 | 100 | B-4 | 20.0 | C-24 | 10.0 | E-5 | 3.0 | D-1/D-4 | 4280/1830 |
| EXAMPLE 74 | J-74 | A-12 | 100 | B-4 | 20.0 | C-1 | 10.0 | E-5 | 3.0 | D-1/D-4 | 4280/1830 |
| EXAMPLE 75 | J-75 | A-12 | 100 | B-4 | 20.0 | C-1/cc-1 | 1.6/1.6 | E-5 | 3.0 | D-1/D-4 | 4280/1830 |
| EXAMPLE 76 | J-76 | A-12 | 100 | B-4 | 20.0 | C-24/cc- | 1.6/1.6 | E-5 | 3.0 | D-1/D-4 | 4280/1830 |
| 1 | | | | | | | | | | | |
| EXAMPLE 77 | J-77 | A-12 | 100 | B-4/B- | 10.0/10.0 | C-1 | 3.2 | E-5 | 3.0 | D-1/D-4 | 4280/1830 |
| 7 | | | | | | | | | | | |
| EXAMPLE 78 | J-78 | A-12 | 100 | B-4/B- | 10.0/10.0 | C-24 | 3.2 | E-5 | 3.0 | D-1/D-4 | 4280/1830 |
| 7 | | | | | | | | | | | |
| EXAMPLE 79 | J-79 | A-12 | 100 | B-4/B- | 10.0/10.0 | C-24 | 3.2 | E-5 | 3.0 | D-1/D-4 | 4280/1830 |
| 3 | | | | | | | | | | | |
| EXAMPLE 80 | J-80 | A-13 | 100 | B-4 | 20.0 | C-1 | 3.2 | E-5 | 3.0 | D-1/D-4 | 4280/1830 |
| EXAMPLE 81 | J-81 | A-14 | 100 | B-4 | 20.0 | C-1 | 3.2 | E-5 | 3.0 | D-1/D-4 | 4280/1830 |
| EXAMPLE 82 | J-82 | A-15 | 100 | B-4 | 20.0 | C-1 | 3.2 | E-5 | 3.0 | D-1/D-4 | 4280/1830 |
| EXAMPLE 83 | J-83 | A-12 | 100 | B-3 | 20.0 | C-1 | 3.2 | E-5 | 3.0 | D-1/D-4 | 4280/1830 |
| EXAMPLE 84 | J-84 | A-12 | 100 | B-7 | 20.0 | C-1 | 3.2 | E-5 | 3.0 | D-1/D-4 | 4280/1830 |
| EXAMPLE 85 | J-85 | A-12 | 100 | B-8 | 20.0 | C-1 | 3.2 | E-5 | 3.0 | D-1/D-4 | 4280/1830 |
| COMPARATIVE | CJ-11 | A-12 | 100 | B-4 | 20.0 | cc-1 | 3.2 | E-5 | 3.0 | D-1/D-4 | 4280/1830 |
| EXAMPLE 11 | | | | | | | | | | | |
| COMPARATIVE | CJ-12 | A-12 | 100 | B-4 | 20.0 | cc-3 | 3.2 | E-5 | 3.0 | D-1/D-4 | 4280/1830 |
| EXAMPLE 12 | | | | | | | | | | | |
| COMPARATIVE | CJ-13 | A-12 | 100 | B-4 | 20.0 | cc-6 | 3.2 | E-5 | 3.0 | D-1/D-4 | 4280/1830 |
| EXAMPLE 13 | | | | | | | | | | | |
| COMPARATIVE | CJ-14 | A-12 | 100 | B-4 | 20.0 | cc-7 | 3.2 | E-5 | 3.0 | D-1/D-4 | 4280/1830 |
| EXAMPLE 14 | | | | | | | | | | | |

### <Formation of Resist Pattern Using Positive Radiation-Sensitive Resin Composition for EUV Exposure>

A composition for forming an underlayer antireflective film (“ARC66” manufactured by Brewer Science, Inc.) was applied onto a 12 inch silicon wafer using a spin coater (“CLEAN TRACK ACT12” manufactured by Tokyo Electron Limited), and then heated at 205° C. for 60 seconds to form an underlayer antireflective film having an average thickness of 105 nm. The positive radiation-sensitive resin composition for EUV exposure prepared above was applied onto the underlayer antireflective film using the spin coater, and subjected to PB at 130° C. for 60 seconds. Thereafter, cooling was performed at 23° C. for 30 seconds to form a resist film having an average thickness of 55 nm. Subsequently, this resist film was exposed with an EUV exposure apparatus (“NXE3300” manufactured by ASML Holding N.V.) with an NA of 0.33 under an illumination condition of conventional illumination (s=0.89), and with a mask of imecDEFECT32FFR02. After the exposure, PEB was performed at 120° C. for 60 seconds. Thereafter, the resist film was subjected to alkaline development using a 2.38 mass % aqueous TMAH solution as an alkaline developer, and after the development, the resist film was washed with water and further dried to form a positive resist pattern (32 nm line-and-space pattern).

### <Evaluation>

The sensitivity and LWR performance of each of resist patterns formed using the positive radiation-sensitive resin compositions for EUV exposure were evaluated according to the following methods. The results are shown in the following Table 7. It is to be noted that a scanning electron microscope (“CG-5000” manufactured by Hitachi High-Tech Corporation) was used for measurement of the resist pattern.

### [Sensitivity]

In formation of the resist pattern using the positive radiation-sensitive resin composition for EUV exposure, an exposure dose at which a 32 nm line-and-space pattern was formed was defined as an optimum exposure dose, and this optimum exposure dose was defined as sensitivity (mJ/cm²). A case where the sensitivity was 30 mJ/cm² or less was evaluated as “good”, and a case where the sensitivity exceeded 30 mJ/cm² was evaluated as “poor”.

### [LWR Performance]

A resist pattern was formed with the mask size adjusted so as to form a 32 nm line-and-space pattern by irradiation with the optimum exposure dose obtained in the evaluation of the sensitivity. The formed resist pattern was observed from above the pattern using the scanning electron microscope. The variation in line width was measured at 500 points in total, the value of 3σ was obtained from the distribution of the measured values, and the value of 3σ was defined as LWR (nm). A smaller value of LWR indicates smaller displacement of the line and better performance. A case where the LWR performance was 4.0 nm or less was evaluated as “good”, and a case where the LWR performance exceeded 4.0 nm was evaluated as “poor”.

TABLE 7

| | RADIATION-SENSITIVE | SENSITIVITY | LWR |
|---|---|---|---|
| | RESIN COMPOSITION | (mJ/cm²) | (nm) |
| EXAMPLE 49 | J-49 | 27.8 | 3.32 |
| EXAMPLE 50 | J-50 | 24.4 | 3.78 |
| EXAMPLE 51 | J-51 | 25.2 | 3.67 |
| EXAMPLE 52 | J-52 | 24.3 | 3.77 |
| EXAMPLE 53 | J-53 | 25.0 | 3.75 |
| EXAMPLE 54 | J-54 | 27.1 | 3.70 |
| EXAMPLE 55 | J-55 | 26.9 | 3.27 |
| EXAMPLE 56 | J-56 | 27.5 | 3.33 |
| EXAMPLE 57 | J-57 | 26.0 | 3.45 |
| EXAMPLE 58 | J-58 | 26.9 | 3.76 |
| EXAMPLE 59 | J-59 | 27.1 | 3.69 |
| EXAMPLE 60 | J-60 | 26.9 | 3.53 |
| EXAMPLE 61 | J-61 | 27.0 | 3.40 |
| EXAMPLE 62 | J-62 | 25.2 | 3.32 |
| EXAMPLE 63 | J-63 | 28.1 | 3.28 |
| EXAMPLE 64 | J-64 | 28.0 | 3.65 |
| EXAMPLE 65 | J-65 | 24.1 | 3.40 |
| EXAMPLE 66 | J-66 | 23.5 | 3.76 |
| EXAMPLE 67 | J-67 | 26.6 | 3.65 |
| EXAMPLE 68 | J-68 | 25.2 | 3.60 |
| EXAMPLE 69 | J-69 | 24.3 | 3.63 |
| EXAMPLE 70 | J-70 | 27.9 | 3.77 |
| EXAMPLE 71 | J-71 | 25.7 | 3.36 |
| EXAMPLE 72 | J-72 | 27.5 | 3.32 |
| EXAMPLE 73 | J-73 | 28.9 | 3.28 |
| EXAMPLE 74 | J-74 | 29.1 | 3.56 |
| EXAMPLE 75 | J-75 | 28.3 | 3.78 |
| EXAMPLE 76 | J-76 | 27.5 | 3.66 |
| EXAMPLE 77 | J-77 | 28.3 | 3.72 |
| EXAMPLE 78 | J-78 | 27.2 | 3.62 |
| EXAMPLE 79 | J-79 | 29.1 | 3.55 |
| EXAMPLE 80 | J-80 | 26.8 | 3.44 |
| EXAMPLE 81 | J-81 | 26.9 | 3.78 |
| EXAMPLE 82 | J-82 | 27.1 | 3.65 |
| EXAMPLE 83 | J-83 | 28.0 | 3.53 |
| EXAMPLE 84 | J-84 | 24.1 | 3.76 |
| EXAMPLE 85 | J-85 | 25.4 | 3.70 |
| COMPARATIVE EXAMPLE 11 | CJ-11 | 35.1 | 4.55 |
| COMPARATIVE EXAMPLE 12 | CJ-12 | 36.3 | 4.34 |
| COMPARATIVE EXAMPLE 13 | CJ-13 | 31.9 | 4.40 |
| COMPARATIVE EXAMPLE 14 | CJ-14 | 32.1 | 4.28 |

As is apparent from the results in Table 7, the radiation-sensitive resin compositions of Examples were superior in sensitivity and LWR performance when used for EUV exposure, whereas the radiation-sensitive resin compositions of Comparative Examples were inferior in characteristics to those of Examples.

[Preparation of Negative Radiation-Sensitive Resin Composition for ArF Exposure, and Formation and Evaluation of Resist Pattern Using this Composition]

### Example 86

First, 100 parts by mass of (A-6) as a resin, 16.0 parts by mass of (B-4) as a radiation-sensitive acid generator, 3.0 parts by mass of (C-1) as the compound (1) (acid diffusion controlling agent), 3.0 parts by mass (solid content) of (E-3) as a high fluorine-content resin, and 3,230 parts by mass of a mixed solvent of (D-1)/(D-2)/(D-3)=70/29/1 (mass ratio) as a solvent were mixed, and the mixture was filtered through a membrane filter having a pore diameter of 0.2 μm to prepare a radiation-sensitive resin composition (J-86).

A composition for forming an underlayer antireflective film (“ARC66” manufactured by Brewer Science, Inc.) was applied onto a 12 inch silicon wafer using a spin coater (“CLEAN TRACK ACT12” manufactured by Tokyo Electron Limited), and then heated at 205° C. for 60 seconds to form an underlayer antireflective film having an average thickness of 105 nm. The negative radiation-sensitive resin composition for ArF exposure (J-86) prepared above was applied onto the underlayer antireflective film using the spin coater, and subjected to PB (prebake) at 90° C. for 60 seconds. Thereafter, cooling was performed at 23° C. for 30 seconds to form a resist film having an average thickness of 90 nm. Subsequently, this resist film was exposed through a mask pattern of 40 nm space and 105 nm pitch using an ArF excimer laser immersion exposure apparatus (“TWINSCAN XT-1900i” manufactured by ASML Holding N.V.) under optical conditions of a numeral aperture (NA) of 1.35 and annular illumination (σ=0.8/0.6). After the exposure, PEB (post exposure bake) was performed at 90° C. for 60 seconds. Thereafter, the resist film was developed with n-butyl acetate as an organic solvent developer, and dried to form a negative resist pattern (40 nm line-and-space pattern).

The resist pattern using the negative radiation-sensitive resin composition for ArF exposure was evaluated in the same manner as in the evaluation of the resist pattern using the positive radiation-sensitive resin composition for ArF exposure. As a result, the radiation-sensitive resin composition of Example 86 was superior in sensitivity, LWR performance, and CDU performance even when a negative resist pattern was formed by ArF exposure.

[Preparation of Negative Radiation-Sensitive Resin Composition for EUV Exposure, and Formation and Evaluation of Resist Pattern Using this Composition]

### Example 87

[A] 100 parts by mass of (A-12) as a resin, [B] 15.0 parts by mass of (B-4) as a radiation-sensitive acid generator, [C] 5.0 parts by mass of (C-28) as an acid diffusion controlling agent, [E] 3.0 parts by mass (solid content) of (E-5) as a high fluorine-content resin, and [D] 6,110 parts by mass of a mixed solvent of (D-1)/(D-4)=70/30 (mass ratio) as a solvent were mixed, and the mixture was filtered through a membrane filter having a pore size of 0.2 μm to prepare a radiation-sensitive resin composition (J-87).

A composition for forming an underlayer antireflective film (“ARC66” manufactured by Brewer Science, Inc.) was applied onto a 12 inch silicon wafer using a spin coater (“CLEAN TRACK ACT12” manufactured by Tokyo Electron Limited), and then heated at 205° C. for 60 seconds to form an underlayer antireflective film having an average thickness of 105 nm. The negative radiation-sensitive resin composition for EUV exposure (J-87) prepared above was applied onto the underlayer antireflective film using the spin coater, and subjected to PB at 130° C. for 60 seconds. Thereafter, cooling was performed at 23° C. for 30 seconds to form a resist film having an average thickness of 55 nm. Subsequently, this resist film was exposed with an EUV exposure apparatus (“NXE3300” manufactured by ASML Holding N.V.) with an NA of 0.33 under an illumination condition of conventional illumination (s=0.89), and with a mask of imecDEFECT32FFR02. After the exposure, PEB was performed at 120° C. for 60 seconds. Thereafter, the resist film was developed with n-butyl acetate as an organic solvent developer, and dried to form a negative resist pattern (40 nm hole, 105 nm pitch).

The resist pattern using the negative radiation-sensitive resin composition for EUV exposure was evaluated in the same manner as in the evaluation of the resist pattern using the positive radiation-sensitive resin composition for EUV exposure. As a result, the radiation-sensitive resin composition of Example 87 was superior in sensitivity and CDU performance even when a negative resist pattern was formed by EUV exposure.

According to the radiation-sensitive resin composition and the resist pattern forming method described above, a resist pattern that is superior in sensitivity to exposure light and excellent in LWR performance and CDU performance can be formed. Therefore, these can be suitably used for a processing process of a semiconductor device in which micronization is expected to further progress in the future.

Obviously, numerous modifications and variations of the present invention are possible in light of the above teachings. It is therefore to be understood that within the scope of the appended claims, the invention may be practiced otherwise than as specifically described herein.

1. A radiation-sensitive resin composition comprising:
an onium salt compound represented by formula (1);
a resin comprising a structural unit having an acid-dissociable group; and
a solvent: 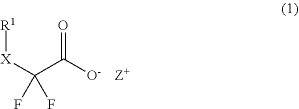
wherein R¹ is a substituted or unsubstituted monovalent organic group having a cyclic structure, or a chain hydrocarbon group having 2 or more carbon atoms,
X is an oxygen atom, a sulfur atom, or —NR^{α}—, wherein R^{α} is a hydrogen atom or a monovalent hydrocarbon group having 1 to 10 carbon atoms, and
Z⁺ is a monovalent onium cation.

2. The radiation-sensitive resin composition according to claim 1, wherein the cyclic structure is at least one selected from the group consisting of an aromatic ring structure, an alicyclic structure, and a heterocyclic structure.

3. The radiation-sensitive resin composition according to claim 2, wherein the aromatic ring structure is at least one selected from the group consisting of structures below:

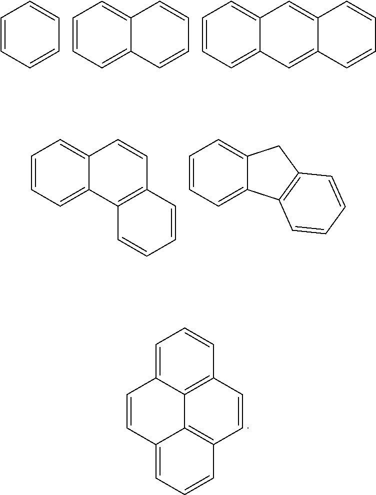

4. The radiation-sensitive resin composition according to claim 2, wherein the alicyclic structure is at least one selected from the group consisting of structures below:

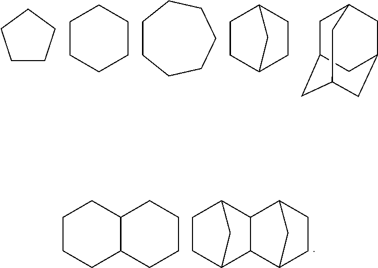

5. The radiation-sensitive resin composition according to claim 2, wherein the heterocyclic structure is at least one selected from the group consisting of structures below:

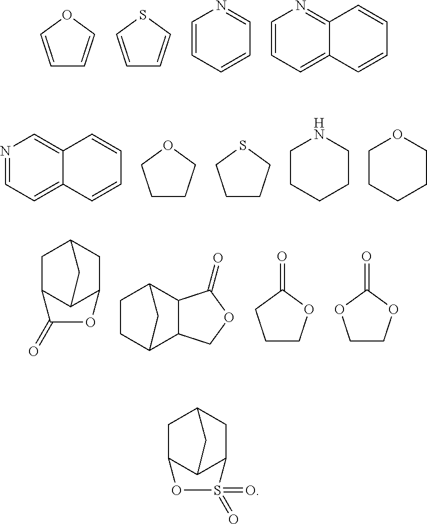

6. The radiation-sensitive resin composition according to claim 1, wherein the onium salt compound comprises a plurality of the cyclic structures, and the plurality of cyclic structures are bonded via a single bond, —O—, —COO—, —OCO—, —CO—, an alkanediyl group, or a combination thereof.

7. The radiation-sensitive resin composition according to claim 1, wherein the onium cation in the formula (1) is a sulfonium cation or an iodonium cation.

8. The radiation-sensitive resin composition according to claim 1, wherein a content of the onium salt compound is 0.1 parts by mass or more and 20 parts by mass or less with respect to 100 parts by mass of the resin.

9. The radiation-sensitive resin composition according to claim 1, further comprising a radiation-sensitive acid generator that generates an acid having a pKa smaller than a pKa of an acid generated from the compound represented by the formula (1).

10. The radiation-sensitive resin composition according to claim 9, wherein a content of the radiation-sensitive acid generator is 0.1 parts by mass or more and 40 parts by mass or less with respect to 100 parts by mass of the resin.

11. A method for forming a resist pattern, comprising:
forming a resist film directly or indirectly on a substrate by applying the radiation-sensitive resin composition according to claim 1;
exposing the resist film; and
developing the exposed resist film.

12. The method according to claim 11, wherein in the exposing of the resist film, the resist film is exposed to ArF excimer laser light, extreme ultraviolet light, or an electron beam.
